SEQUENCE LISTING (22 sequences)

SEQ ID NO: 1 - DNA, 21 bp, Artificial Sequence
  Synthetic Construct
ctcttctttcttttctttttg

SEQ ID NO: 2 - DNA, 21 bp, Artificial Sequence
  Synthetic Construct
gagaagaaagaaaagaaaaac

SEQ ID NO: 3 - DNA, 15 bp, Artificial Sequence
  Synthetic Construct
tgcggtctttggcgg

SEQ ID NO: 4 - DNA, 15 bp, Artificial Sequence
  Synthetic Construct
acgccagaaaccgcc

SEQ ID NO: 5 - DNA, 39 bp, Artificial Sequence
  Synthetic Construct
cctatccctgtgtgcctgcctatccgttgcgtgtctcag

SEQ ID NO: 6 - DNA, 69 bp, Artificial Sequence
  Synthetic Construct
tgtatcactatgacgcgcctgactctctgactgagacacgcaacggataggcaggcacacagggatagg

SEQ ID NO: 7 - DNA, 20 bp, Artificial Sequence
  Synthetic Construct
ccatctcatccctgcgtgtc

SEQ ID NO: 8 - DNA, 20 bp, Artificial Sequence
  Synthetic Construct
cctatcccctgtgtgccttg

SEQ ID NO: 9 - DNA, 26 bp, Artificial Sequence
  Synthetic Construct
tgtagtcgccatgtaactcatcggca

SEQ ID NO: 10 - DNA, 188 bp, Artificial Sequence
  Synthetic Construct
ccatctcatccctgcgtgtcccatctgttccctccctgtctcagtgtcattgatgtagtcgccatgtaactcatcggcaataggctgtaaatccacatgtacgacaatccgcgtcagtttaccgcttaacatatcgaagaacggctgagacacgcaacaggggataggcaaggcacacaggggatagg

SEQ ID NO: 11 - DNA, 39 bp, Artificial Sequence; identical to SEQ ID NO: 5
  Synthetic Construct

SEQ ID NO: 12 - DNA, 100 bp, Artificial Sequence
  Synthetic Construct
atgtgtattaatgatgagccgccaggagcacctccatctatttttctcgggcctagctgactgagacacgcaacgggataggcaggcacacagggatagg

SEQ ID NO: 13 - DNA, 100 bp, Artificial Sequence
  Synthetic Construct
actatgagagtgttccacacaccgcgttgccctacactcgctgccgactcaatggtctgactgagacacgcaacgggataggcaggcacacagggatagg

SEQ ID NO: 14 - DNA, 79 bp, Artificial Sequence
  Synthetic Construct
ccccctcttctttcttttgtttttcttttctttcttctcctgagacacgcaacgggataggcaggcacacagggatagg

SEQ ID NO: 15 - DNA, 20 bp, Artificial Sequence
  Synthetic Construct
ccatctcatccctgcgtgtc

SEQ ID NO: 16 - DNA, 20 bp, Artificial Sequence
  Synthetic Construct
gacacgcagggatgagatgg

SEQ ID NO: 17 - DNA, 20 bp, Artificial Sequence
  Synthetic Construct
ccatctcatccctgcgtgtc

SEQ ID NO: 18 - DNA, 20 bp, Artificial Sequence
  Synthetic Construct
cctatcccctgtgtgccttg

SEQ ID NO: 19 - DNA, 100 bp, Artificial Sequence
  Synthetic Construct
cctatcccctgtgtgccttgtcagctaggcccgagaaaaatagatggaggtgctcctggcggctcatcattaatacacatgacacgcagggatgagatgg

SEQ ID NO: 20 - DNA, 20 bp, Artificial Sequence
  Synthetic Construct
ccatctcatccctgcgtgtc

SEQ ID NO: 21 - DNA, 20 bp, Artificial Sequence
  Synthetic Construct
cctatcccctgtgtgccttg

SEQ ID NO: 22 - DNA, 100 bp, Artificial Sequence
  Synthetic Construct
ccatctcatccctgcgtgtcatgtgtattaatgatgagccgccaggagcacctccatctatttttctcgggcctagctgacaaggcacacaggggatagg

(19) United States
(12) Patent Application Publication
(10) Pub. No.: US 2013/0053252 A1
(43) Pub. Date: Feb. 28, 2013

(54) NUCLEIC ACID AMPLIFICATION AND SEQUENCING BY SYNTHESIS WITH FLUOROGENIC NUCLEOTIDES

(71) Applicants: Xiaoliang Sunney Xie, Lexington, MA (US); Peter A. Sims, Cambridge, MA (US); William J. Greenleaf, Belmont, MA (US); Haifeng Duan, Cambridge, MA (US)

(72) Inventors: Xiaoliang Sunney Xie, Lexington, MA (US); Peter A. Sims, Cambridge, MA (US); William J. Greenleaf, Belmont, MA (US); Haifeng Duan, Cambridge, MA (US)

(73) Assignee: President & Fellows of Harvard College, Cambridge, MA (US)

(21) Appl. No.: 13/498,072

(22) PCT Filed: Sep. 24, 2010
(86) PCT No.: PCT/US10/50215

§ 371 Date: Oct. 22, 2012

Related U.S. Application Data
(60) Provisional application No. 61/245,810, filed on Sep. 25, 2009.
(60) Provisional application No. 61/307,060, filed on Feb. 23, 2010.
(60) Provisional application No. 61/332,997, filed on May 10, 2010.
(60) Provisional application No. 61/370,261, filed on Aug. 3, 2010.

(51) Int. Cl. C40B 20/00; C07H 19/20; C40B 50/06; C40B 60/10; G01N 21/64; C12P 19/34

(52) U.S. Cl. 506/2; 435/6.1; 435/91.2; 506/26; 506/38; 536/26.22

(57) ABSTRACT

In general, the invention features methods and systems for sequencing of nucleic acids based on the measurement of the incorporation of fluorogenic nucleotides in microreactors. The invention provides numerous advantages over previous systems such as unambiguous determination of sequence, fast cycle time, long read lengths, low overall cost of reagents, low instrument cost, and high throughput. The invention also features methods and kits for nucleic acid amplification. The amplification and sequencing aspects of the invention may or may not be employed in conjunction with one another. The invention also features fluorogenic nucleotides that may be used in the sequencing methods of the invention.

### CROSS-REFERENCE TO RELATED APPLICATIONS

This application claims benefit of U.S. Provisional Application No. 61/245,810, filed Sep. 25, 2009, U.S. Provisional Application No. 61/307,060, filed Feb. 23, 2010, U.S. Provisional Application No. 61/332,997, filed May 10, 2010, and U.S. Provisional Application No. 61/370,261, filed Aug. 3, 2010, each of which is hereby incorporated by reference.

### STATEMENT AS TO GOVERNMENT SPONSORSHIP

This invention was made with government support under P10D0002, ROI HG005097-01, and 1RC2HG005613-01 awarded by the National Institutes of Health. The government has certain rights in the invention.

### BACKGROUND OF THE INVENTION

The invention relates to the fields of high throughput nucleic acid sequencing and amplification.

High-throughput, cost-effective DNA and RNA sequencing promises to usher in a new era of personalized medicine. However, a dramatic reduction in cost and increase in speed are needed for mass-market genetic analysis to benefit human health.

Accordingly, there is a need for new methods, kits, reagents, and devices for rapid and accurate nucleic acid sequencing and amplification.

### SUMMARY OF THE INVENTION

In general, the invention features methods and systems for sequencing of nucleic acids based on the measurement of the incorporation of fluorogenic nucleotides in microreactors. The invention provides numerous advantages over previous systems such as unambiguous determination of sequence, fast cycle time, long read lengths, low overall cost of reagents, low instrument cost, and high throughput. The invention also features methods and kits for nucleic acid amplification. The amplification and sequencing aspects of the invention may or may not be employed in conjunction with one another.

In one aspect, the invention provides a method for sequencing a nucleic acid by immobilizing a single target nucleic acid or a number of substantially identical copies of the target nucleic acid within a microreactor, then providing a mixture in solution phase to this microreactor, which is optionally sealed, e.g., with a water-immiscible liquid such as a silicone, hydrocarbon, or fluorocarbon oil or by pressing the microreactors against a membrane or solid substrate. This mixture includes a nucleic acid replicating catalyst (e.g., DNA polymerase, RNA polymerase, ligase, RNA-dependent RNA polymerase, or reverse transcriptase), and a first nucleotide species having a label that is substantially non-fluorescent until after incorporation of the first nucleotide into a nucleic acid based on complementarity to the target nucleic acid. The mixture in solution phase, e.g., having a volume of 0.0001 fL-100000 fL, is disposed in a microreactor, and template-dependent replication of the target nucleic acid is allowed to occur. The target nucleic acid is then sequenced by detecting, after a suitable time, fluorescence generated from this first label as a result of the incorporation of the first nucleotide during template-dependent replication. If this included nucleotide species is not complementary to the target nucleic acid sequence, negligible fluorescence is generated. However, if the target nucleic acid sequence contains multiple sequential bases that are complementary to this first nucleotide species, then the generated fluorescence signal will be larger than that expected for a single nucleotide incorporation. In this way homopolymer stretches in the target nucleic acid can be efficiently sequenced. After quantification of fluorescence signal, the solution within the microreactor is then exchanged for a different mixture in solution phase, which includes a nucleic acid replicating catalyst (e.g., DNA polymerase, RNA polymerase, ligase, RNA-dependent RNA polymerase, or reverse transcriptase), and a second nucleotide species having a label that is substantially non-fluorescent until after incorporation of the second nucleotide into a nucleic acid based on complementarity to the target nucleic acid. If this second nucleotide species is complementary to the target nucleic acid, fluorescent label is generated by the nucleic acid replicating catalyst, otherwise negligible signal is generated. These steps are repeated for all nucleotide species serially and repeatedly, allowing full determination of the target nucleic acid sequence. The labels attached to each different nucleotide employed in the methods may be the same or different. Liquid exchange may occur through unsealing sealed microreactors, removing the liquid contents, introducing a new mixture in solution phase, and resealing the microreactors.

In some embodiments, the nucleic acid replicating catalyst is tightly bound to the nucleic acids being sequenced, and therefore need not be reintroduced in subsequent cycles of sequencing.

The detection step may be repeated as desired to continue sequencing the target nucleic acid by detecting incorporation of the next nucleotide, e.g., for at least 10, 25, 100, 300, 1000, or 10,000 base pairs.

In certain embodiments, the mixture in solution phase further includes an activating enzyme that renders the label fluorescent. Examples of activating enzymes include an alkaline phosphatase, acid phosphatase, galactosidase, horseradish peroxidase, phosphodiesterase, phosphotriesterase, pyruvate kinase, lactic dehydrogenase, maltose phosphorylase, glucose oxidase, lipase, and combinations thereof. Activating enzymes may be immobilized on the surface of a microreactor or on a bead disposed in the microreactor.

In other embodiments, the mixture in solution phase further includes non-hydrolyzable nucleotide substrates that inhibit misincorporation of the labeled nucleotide substrate species by binding to the replicating catalyst, e.g., polymerase, on nucleic acid molecules, in which the template base is not complementary to the labeled nucleotide substrate. In this way, these non-hydrolyzable nucleotide substrates block the labeled substrate from binding with the replicating catalyst, e.g., polymerase, and thereby reduce or prevent misincorporation events. Non-hydrolyzable nucleotide analogs are well known in the art.

In other embodiments, a second mixture in solution phase containing an unlabeled nucleotide species including the first base is introduced into the microreactor and template-dependent replication is allowed to proceed until the sequencing cycle is complete. The second mixture may further include three non-hydrolyzable nucleotide species, with second, third, and fourth bases, where the first, second, third, and fourth bases are different.

In other embodiments, the label is photobleached after fluorescence detection. The label may also be a phosphate label that is cleaved from the nucleotide during incorporation.

DNA, RNA or combinations thereof may be sequenced in the methods of the invention. For DNA or RNA, a primer may be employed. The methods of the invention may also be multiplexed to determine the sequence of more than one target nucleotide at the same time or sequentially.

In certain embodiments, the nucleic acid is immobilized either to the microreactor or to a bead within the microreactor using any of a number of methods (such as biotin-streptavidin, antigen-antibody affinity, covalent attachment, or nucleic acid complementarity). For example, the nucleic acid may be attached to a micron-sized bead disposed in the microreactor or to a lid of the microreactor. When a bead is employed, it may be magnetic and immobilized in a microreactor using a magnetic field. The target nucleic acid or plurality of copies may be immobilized in a spatial pattern, e.g., via biotin, on a surface of a microreactor. The pattern may be formed by spatially selective exposure to air plasma and subsequent coupling of a binding moiety, e.g., biotin or an oligonucleotide, or my spatially selective application of such a binding moiety.

The methods of the invention may also be employed with reversibly terminated nucleotides and with enzymatic signal amplification techniques as described herein.

The mixture in solution phase may further include an exonuclease, where a plurality of first labels is produced as a result of incorporation of the nucleotide and subsequent excision by the exonuclease. In such embodiments, the nucleotide may not be capable of extension. In other embodiments, the nucleotide excised is replaced with a nucleotide that is resistant to exonuclease excision and optionally reversibly terminated, e.g., an optionally reversibly terminated α-phosphorothioate.

The target nucleic acid may be reversibly bound to a bead when it is introduced into the microreactor. In certain embodiments, the microreactors include bound oligonucleotides, and a nucleic acid complementary, e.g., a single copy, to the target nucleic acid and reversibly bound to a bead is introduced into the microreactor. The complementary nucleic acid binds to a bound oligonucleotide, which is extended via template-dependent replication, thereby immobilizing the target nucleic acid in the microreactor. Such embodiments may further include performing template dependent replication of the target nucleic acid to produce from the bound oligonucleotides a plurality of copies of the target nucleic acid bound to the microreactor. The bead may be removed once the complementary nucleic acid is bound to the microreactor.

In certain embodiments, the plurality of copies is produced by rolling circle amplification (with or without hyperbranching), which may be followed by PCR amplification. The plurality of copies also may or may not be a concatemer.

In other embodiment, the temperature of the microreactor is reduced, e.g., to 15° C. or lower, when a fluorogenic nucleotide species is introduced. Subsequently, the temperature of the microreactor may be raised, e.g., to 20° C. or higher, during incorporation of the nucleotide species in template-dependent replication. If a lid is present, it may be closed prior to an increase in temperature. Template-dependent replication may or may not employ thermocycling.

The sequencing methods may also be employed with a population of single target nucleic acids or a population of pluralities of copies of the target nucleic acids, wherein each single target nucleic acid or plurality of copies of the target nucleic acid is immobilized in one of a plurality of microreactors. The plurality of microreactors may be super-Poisson loaded with the population of single target nucleic acids or population of pluralities of copies of the target nucleic acids. In one method of super-Poisson loading, the pluralities of copies of the target nucleic acids are concatemers sized so that only one concatemer is disposed in one of the plurality of microreactors. In another method of super-Poisson loading, each single target nucleic acid or plurality of copies of the target nucleic acid is bound to a bead sized so that only one bead is disposed in one of the plurality of microreactors. In a further method of super-Poisson loading, at least two repetitions of Poisson loading the population of single target nucleic acids, or complement thereof, or population of pluralities of copies of the target nucleic acids or complement thereof into a subset of the plurality of microreactors so that subsequent loading of the subset is prevented are performed. For example, each repetition includes loading a nucleic acid complementary to the target nucleic acid to the subset of microreactors and extending substantially all (or at least 70%, 75%, 80%, 85%, 90%, 95%, or 99%) of an oligonucleotide bound to a surface of the subset of microreactors by template dependent replication to produce the target nucleic acids. In another example, each repetition includes adding the population of plurality of copies of the target nucleic acid to the subset of microreactors, wherein the copies comprise a binding moiety that binds to moieties bound to a surface of the microreactors, and wherein, for each plurality and microreactor, the number of copies is sufficient to bind to substantially all (or at least 70%, 75%, 80%, 85%, 90%, 95%, or 99%) of the moieties bound to the surface. Alternatively, a repetition may include binding a number of binding sites on the surface of the microreactor and then treating the microreactor to prevent further binding of nucleic acids.

The immobilizing step may include adding a nucleic acid complementary to the target nucleic acid to the microreactor and extending an oligonucleotide bound to a surface of the microreactor by template dependent replication to produce the target nucleic acid or adding the plurality of copies of the target nucleic acid to the microreactor, wherein the copies include a binding moiety that binds to moieties bound to a surface of the microreactor, and wherein the number of copies is sufficient to bind to substantially all of the moieties bound to the surface.
In methods where nucleic acids are bound to oligonucleotide on the surface of a microreactor, the oligonucleotide may be a PCR primer, or it may melt from a nucleic acid complementary to the target nucleic acid at 35° C. or higher.

The plurality of copies of the target nucleic acid may be employed in the sequencing and may be produced by any of the amplification methods described herein.

In one embodiment, the method for sequencing a nucleic acid includes immobilizing in a microreactor a single target nucleic acid or a plurality of copies of the target nucleic acid; cooling the microreactor to 15° C. or lower; introducing to the microreactor a mixture in solution phase including a nucleic acid replicating catalyst, and a single species of nucleotide having a first base and a first label that is substantially non-fluorescent until after incorporation of the nucleotide into a nucleic acid based on complementarity to the target nucleic acid; sealing the microreactor and heating the microreactor to 20° C. or higher; allowing template-dependent replication of the target nucleic acid or the plurality of copies of the target nucleic acid; sequencing the target nucleic acid by detecting incorporation of the nucleotide during template-dependent replication by detecting fluorescence emission resulting from the first label; repeating the previous steps sequentially with a second single nucleotide species having a second base and a second label that is substantially non-fluorescent until incorporation of the second nucleotide into the nucleic acid based on complementarity to the target nucleic acid, a third single nucleotide species having a third base and a third label that is substantially non-fluorescent until incorporation of the third nucleotide into the nucleic acid based on complementarity to the target nucleic acid; and a fourth single nucleotide species having a fourth base and a fourth label that is substantially non-fluorescent until incorporation of the fourth nucleotide into the nucleic acid based on complementarity to the target nucleic acid, wherein any two of the first, second, third and fourth labels are the same or different, and the first, second, third, and fourth bases are different.

In another aspect, the invention features a method of amplifying a nucleic acid by providing a single copy of a first nucleic acid (e.g., single or double stranded) having first and second ends; immobilizing the first nucleic acid via the first end to a bead; immobilizing the second end of the nucleic acid to a surface of a microreactor; and amplifying, e.g., by polymerase chain reaction or ligase chain reaction, the first nucleic acid to produce a plurality of amplicons having first and second ends, wherein the plurality of amplicons binds to the surface of the microreactor via the second ends or to the bead via the first ends. Alternatively, the nucleic acid may be immobilized to the microreactor without the use of a bead.

Alternatively, the invention features a method of amplifying a nucleic acid by providing a single copy of a first nucleic acid having first and second ends; optionally immobilizing the first nucleic acid via the first end to a bead; immobilizing the second end of the first nucleic acid to one of a plurality of complementary oligonucleotides bound to a surface of a microreactor; extending the oligonucleotide by template dependent replication to produce a second nucleic acid bound to the surface of the microreactor; and amplifying the second nucleic acid to produce a plurality of amplicons extended from said plurality of oligonucleotides bound to the surface of the microreactor. In this embodiment, the bead may be removed once the complementary oligonucleotide is delivered to microreactor. In certain embodiments, substantially all (or at least 70%, 75%, 80%, 85%, 90%, 95%, or 99%) of the oligonucleotides are extended. The oligonucleotide may be a PCR primer, or it may melt from a nucleic acid complementary to the target nucleic acid at 35° C. or higher. In another embodiment, the oligonucleotides not extended are treated to prevent extension, e.g., by degradation or cleavage from the surface.

Another amplification method includes providing a single copy of a first circular nucleic acid; immobilizing the first nucleic acid to one of a plurality of complementary oligonucleotides bound to a surface of a microreactor or a bead; extending the oligonucleotide by rolling circle amplification to produce a second nucleic acid bound to the surface of the microreactor or bead; and amplifying, e.g., by linear or nonlinear rolling circle amplification, the second nucleic acid to produce a plurality of amplicons extended from the plurality of oligonucleotides bound to said surface of said microreactor. This method may further include amplifying the product by PCR.

In embodiments of the amplification methods, a first oligonucleotide adaptor is coupled to the first end of the first nucleic acid, e.g., by ligation, and a second oligonucleotide adaptor is coupled to the second end of the first nucleic acid, e.g., by ligation, wherein the first adaptor includes a moiety that optionally binds to the bead, and the second adaptor includes a moiety that binds to the surface of the microreactor. The first and second adaptors may also include nucleotide sequences to which forward and reverse primers for PCR hybridize.

The bead may include an oligonucleotide having a sequence to which the first end of the first nucleic acid hybridizes. Similarly, the surface of the microreactor may include an oligonucleotide having a sequence to which the second end of the first nucleic acid hybridizes.

Amplifying may occur by any suitable method, e.g., PCR, LCR, RCA, or HRCA.

The first nucleic acid is, for example, isolated from a library or biological sample. The library or biological sample may be fragmented to produce a plurality of nucleic acids including the first nucleic acid. The method may also be repeated for a plurality of single copies of nucleic acids. For example, the method may occur simultaneously for a plurality of nucleic acids, wherein each nucleic acid is immobilized in a separate microreactor.

In certain embodiments, the microreactor and bead are sized so that only one bead is immobilized in the microreactor.

The amplicons may be bound to the surface of the microreactor or to the bead, and the bead may be removed from the microreactor after amplification.

The microreactor may be sealed after delivery of the nucleic acid, e.g., with a water-immiscible liquid or by pressing the microreactors against a membrane or solid substrate. In addition, single copies of nucleic acids may also be delivered to the microreactor by methods other than beads, e.g., solution phase delivery of a dilute solution.

In certain embodiments, additional target nucleic acids cannot be immobilized in the microreactor after amplification. These methods may be employed in super-Poisson loading of a plurality of microreactors. For example, single nucleic acids can be Poisson loaded in a subset of a plurality of microreactors and amplified, and this process can be repeated to achieve super-Poisson loading.

Any of the amplification methods described herein may be employed to produce a plurality of nucleic acids for use in the sequencing methods provided herein, e.g., employing fluorescent, chemiluminescent, or electrical detection. In preferred embodiments, the amplification and sequencing occur in the same microreactor.

The invention further features a system for sequencing a nucleic acid that includes a plurality of microreactors each of which is capable of holding a different set of immobilized, substantially identical target nucleic acids for sequencing, and a solution phase mixture of a nucleic acid replicating catalyst, and a nucleotide that has a label that is substantially non-fluorescent until after incorporation of that nucleotide into a nucleic acid based on complementarity to the target nucleic acid; and a fluorescent microscope for imaging the plurality of microreactors to sequence target nucleic acids in the microreactors by the methods described herein. The system may include a light source, e.g., the excitation source of the microscope, capable of photobleaching the label after detection.

The system may further include a fluidic delivery system capable of delivering liquids to each of the plurality of microreactors and/or a light source capable of eliciting fluorescence from the label for detection. This fluidic system may be capable of performing emulsion PCR (Dressman (2003) Proc. Natl. Acad. Sci. USA 100:8817; Brenner et al. (2000) Nat. Biotech. 18:630), bridge PCR (Bentley et al. Nature, 2008, 456, 54), other solid-phase PCR, or linear nucleic acid amplification to generate distinct populations of substantially identical nucleic acids and immobilize them within a microreactor. This fluidic system may also be capable of purifying and amplifying nucleic acids from cells for sequencing. For example, the system may be capable of isolating a single cell, purifying RNA or DNA from the cell, and amplifying this nucleic acid for subsequent sequencing. This fluidic system may also be capable of sealing the array of microreactors using applied pressure. In particular, the plurality of microreactors may further include a control layer, pressurization of which conformally seals the microreactors against a flat surface. In such embodiments, the system further includes a pressure source. The system may also include a temperature controller capable of reducing the temperature of the microreactors below room temperature and capable of increasing the temperature of the microreactors to perform template dependent nucleic acid replication. The temperature controller may also be capable of thermocycling the plurality of microreactors so that nucleic acids present are amplified. The system may further include computer software (on a physical memory) or hardware to control the operation of the individual components. In particular, computer software or hardware may be present that controls the temperature of the microreactors during introduction of a labeled nucleotide, e.g., to 15° C. or below; during sealing of the array; during template dependent replication, e.g., to 20° C. or above; and any combination thereof.

Microreaders may be fabricated from poly(dimethylsiloxane) (PDMS) or a combination of PDMS and glass. These devices may be coated with a fluorocarbon polymer (e.g., CYTOP) and a polyethyleneoxide-polypropyleneoxide block copolymer, such as a poloxamer (e.g., Pluronic F-108) or poloxamine. Alternatively, the reactor surface may be coated with protein-based passivation agents (e.g., bovine serum albumen or casein). PDMS microreactors may also be treated with a fluorocarbon fluid such as Fluorinert (e.g., FC-43 or FC-770). Glass surfaces may be silanized for surface passivation (e.g., 1H, 1H, 2H, 2H-perfluorooctyltrichlorosilane or [tris(trimethylsiloxy)silylethyl]dimethylchlorosilane) and/or to allow surface conjugation of the nucleic acid or other components of the mixture (e.g., using 3-mercaptopropyltrimethoxysilane). Additionally, the reactor surface may be passivated by covalent coupling of polyethylene glycol (PEG) to the surface.

The microreactors may be patterned with a binding moiety, e.g., biotin or an oligonucleotide.

The system may also include a stage that is capable of moving the plurality of microreactors relative to the fluorescence microscope, so that a first portion of the plurality of microreactors is imaged. The fluidic delivery system may also be capable of delivery fluids to a second portion of the plurality of microreactors while the first portion of the plurality of microreactors is imaged. In other embodiments, a third portion of the plurality of microreactors is undergoing template-dependent replication, while fluids are delivered to the second portion of the plurality of microreactors, and the first portion of the plurality of microreactors is imaged.

The invention also features kits including a nucleic acid replicating catalyst (e.g., DNA polymerase, RNA polymerase, ligase, RNA-dependent RNA polymerase, or reverse transcriptase), four nucleotides each having a label that is substantially non-fluorescent until after incorporation of the nucleotide into a nucleic acid based on complementarity to the target nucleic acid, and an activating enzyme that renders the label fluorescent (e.g., an alkaline phosphatase, acid phosphatase, galactosidase, horseradish peroxidase, phosphodiesterase, phosphotriesterase, pyruvate kinase, lactic dehydrogenase, maltose phosphorylase, glucose oxidase, lipase, or combination thereof). The four nucleotides are typically sufficient to allow complete sequencing of a naturally occurring nucleic acid, e.g., including A, T or U, C, and G. Each nucleotide may have a distinct label, or any two or more of the nucleotides may include the same label.

In a related aspect, the invention provides a kit including a plurality of microreactors that are each capable of holding an immobilized single target nucleic acid, a mixture in solution phase of reagents for template dependent replication of the single target nucleic acid, and a bead functionalized to bind to the single target nucleic acid; a plurality of beads that are each capable of binding a nucleic acid and being disposed within one of the microreactors; and reagents for template dependent replication of the nucleic acid. The kit may also include a water-immiscible liquid for sealing the microreactors. The microreactors may include bound oligonucleotides or a spatially patterned binding moiety, e.g., biotin. Other exemplary microreactors, beads, and reagents are described herein.

The invention also provides a compound having the formula:

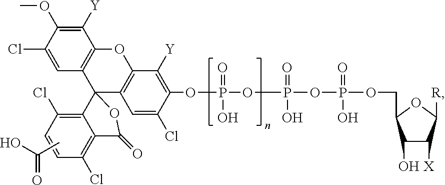

wherein n is 0 to 4, R is a nucleoside base, X is H, OH, or OMe, and Y is H or Cl, or a salt thereof.

The invention also features a compound having the formula:

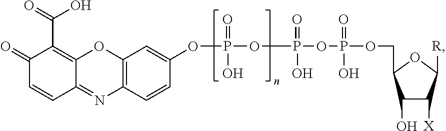

wherein n is 0 to 4, R is a nucleoside base, and X is H, OH, or OMe, or a salt thereof.

By “adaptor” is meant a chemical moiety capable of covalently binding to the 5′ or 3′ end of a nucleic acid and having a binding moiety capable of covalently or noncovalently attaching the nucleic acid to a solid surface, e.g., bead or microreactor.

By “amplicon” is meant a product of template-dependent nucleic acid replication. Depending on the technique employed, an amplicon may have the same sequence or the complementary sequence of a nucleic acid being replicated. Amplicons may also include only a portion of the sequence or complement of the nucleic acid being replicated or additional moieties not found in the nucleic acid being replicated, e.g., via primers or nucleotides employed in replication.

By “amplifying” is meant producing a plurality of copies of a nucleic acid, either substantially identical in sequence, complementary in sequence, or both, by a template-dependent replicating process.

By “bead” is meant any particle that does not dissolve during nucleic acid sequencing or amplification and that is capable of binding a nucleic acid, either covalently or noncovalently. Beads may be magnetic or nonmagnetic.

By “biological sample” is meant any sample of biological origin containing nucleic acid. Sources of sample include whole organisms (e.g., single cellular organisms and viruses), tissues, and culture samples.

By “capable of extension” is meant capable of having a nucleotide added through template-dependent replication. For example, a DNA or RNA nucleotide is capable of extension. Once a reversibly terminated or dideoxy nucleotide is incorporated into a primer-template nucleic acid molecule, subsequent primer extension is not possible.

By “fluorogenic” or “substantially non-fluorescent” is meant not emitting a significant amount of fluorescence at a given wavelength until after a chemical reaction has occurred.

By “incorporation” of a nucleotide into a nucleic acid is meant the formation of a chemical bond, e.g., a phosphodiester bond, between the nucleotide and another nucleotide in the nucleic acid. For example, a nucleotide may be incorporated into a replicating strand of DNA via formation of a phosphodiester bond. Other types of bonds may be formed if non-naturally occurring nucleotides are employed.

By a “microreactor” is meant a vessel having a volume such that a light microscope can detect the buildup of a freely diffusing fluorophore using a photon detector.

By “nucleotide” is meant a natural or synthetic ribonucleosidyl, 2′-deoxyribonucleosidyl radical, 2′-O-methyl ribonucleosidyl, Locked Nucleic Acid, peptide nucleic acid, glycerol nucleic acid, morpholino nucleic acid, or threose nucleic acid connected, e.g., via the 5′, 3′ or 2′ carbon of the radical, to a phosphate group and a base. The nucleotide may include a purine or pyrimidine base, e.g., cytosine, guanine, adenine, thymine, uracil, xanthine, hypoxanthine, inosine, orotate, thioinosine, thiouracil, pseudouracil, 5,6-dihydrouracil, and 5-bromouracil. The purine or pyrimidine may be substituted as is known in the art, e.g., with halogen (i.e., fluoro, bromo, chloro, or iodo), alkyl (e.g., methyl, ethyl, or propyl), acyl (e.g., acetyl), or amine or hydroxylprotecting groups. In certain embodiments when DNA is being sequenced, the nucleotides employed are dATP, dCTP, dGTP, and dTTP. In other embodiments when RNA is being sequenced, the nucleotides employed are ATP, CTP, GTP, and UTP. A target DNA sequence can also be sequenced with riboside bases using RNA polymerase, and a target RNA sequence can also be sequenced with deoxyriboside bases using reverse transcriptase. The term includes moieties having a single base, e.g., ATP, and moieties having multiple bases, e.g., oligonucleotides.

By “nucleotide replicating catalyst” is meant any catalyst, e.g., an enzyme, that is capable of producing a nucleic acid that is complementary to a target nucleic acid. Examples include DNA polymerases, RNA polymerases, reverse transcriptases, ligases, and RNA-dependent RNA polymerases.

By “rolling circle amplification” is meant amplification of a circular nucleic acid with a strand-displacing nucleic acid replicating catalyst.

By “sequencing” a nucleic acid is meant identification of one or more nucleotides in, or complementary to, a target nucleic acid. Sequencing may include determination of the individual bases in sequence, determination of the presence of an oligonucleotide sequence, or determination of the class of nucleotide present, e.g., member of A-T, A-U, or G-C pair, or purine base or pyrimidine base.

Other features and advantages of the invention will be apparent from the following drawings, detailed description, and the claims.

### BRIEF DESCRIPTION OF THE DRAWINGS

FIG. 1: Fluorogenic sequencing using a coupled enzyme assay. A) A strand of immobilized DNA with a polymerase bound, ready to add the next base to the primer strand of the DNA. This strand represents one of the population of substantially identical strands of DNA immobilized in the reaction chamber. Phosphates are represented by small circles, and fluorophores are represented by large circles. Semi-transparent circles are dark because they are conjugated to one or more phosphates. B) The polymerase recognizes the correct, complementary nucleotide to add to the primer strand and binds it. C) The polymerase adds the nucleotide, generating a natural incorporated base as well as a dark fluorophore conjugated to two phosphates. D) A phosphatase cleaves one of these two phosphates, and then E) cleaves the other, generating a fluorescent molecule that can be detected. F) Upon detection of this incorporated base, the fluorescent tag and phosphates exit the reaction volume. The phosphatase and polymerase are also optionally exchanged (signified by their transparency). G) buffer containing another base, along with polymerase and phosphatase, is introduced to probe the next base in the sequence. H) steps A)-G) are repeated serially with each species of nucleotide, allowing full sequencing of the immobilized DNA.

FIG. 2A: Valve-based sealing of PDMS microreactors. The PDMS microreactor includes a control layer (A) which allowed for reversible sealing of the reaction chambers upon application of pressure (B).

FIG. 2B: Two-layer PDMS microfluidic device for on-chip PCR consisting of a microreactor array-containing flow layer and a pressurizable control layer with a membrane for sealing the array. Both the control layer and the flow layer can be pressurized with water to prevent evaporation of the microrcactors during thermocycling.

FIG. 3: A fluorescence image of dye trapped in oil covered PDMS microreactors (5 μm diameter).

FIG. 4: One reversibly terminated nucleotide (with red polygons representing the reversible terminator moiety on the 3′ end) is incorporated into a homopolymeric DNA sequence, generating a fluorescent label (A-F). However, upon incorporation of the reversible terminator, no subsequent incorporations of the base are possible, even though they are complementary to the template strand. Upon removal of the nucleotides and the reversible terminator moiety (G), further incorporation of nucleotides into the homopolymeric region can occur (H), one nucleotide at a time.

FIG. 5: Small red polygons in the backbone of the DNA represent linkages that are resistant to the action of the exonuclease (for example phosphorothioate linkages). Fluorogenic nucleotides are incorporated into the DNA generating fluorescent product (A-F). Exonuclease then digests this newly incorporated base (G) leading to subsequent incorporations of the fluorogenic nucleotide (H) and generation of multiple fluorescent labels (I). The solution is then replaced with nucleotides which, when incorporated, generate DNA that is resistant to exonuclease digestion (J). One of these nucleotides is incorporated (K), and sequencing of the next base, with enzymatic amplification, can occur (L).

FIG. 6: Scheme for scanning microreactors in a rectilinear pattern.

FIG. 7: Scheme for simultaneous detection of microreactors in a rectilinear pattern.

FIG. 8: Scheme for amplification of a single copy of a nucleic acid in a microreactor.

FIG. 9: Scheme for amplification of a single copy of a nucleic acid in a microreactor.

FIG. 10: Scheme for pre-amplification by linear, rolling circle amplification and in-microreactor amplification with PCR.

FIG. 11: Scheme for hyperbranched rolling circle amplification.

FIG. 12: Scheme for rolling circle amplification for direct sequencing with PCR amplification.

FIGS. 13A-C: Schematic depictions of surface preparations for super-Poisson loading of microreactors.

FIG. 14: Work flow for thermocycle fluorogenic DNA sequencing in PDMS microreactors. In this case, DNA template-coated beads are immobilized in each microreactor.

FIGS. 15A-E: A) A schematic depiction of a thermocycler for use with the invention; B) exemplary thermal cycles achievable with this device; and C)-E) photographs of a thermocycler with a PDMS microreactor array seated on it.

FIG. 16: An exemplary microreactor fabrication procedure. Polystyrene beads are close-packed onto a flat glass surface. Polydimethylsiloxane (PDMS) is poured and cured onto these beads and then removed. The impregnated beads are removed mechanically, and the coupled-enzyme reaction mixture is placed between the patterned PDMS and a PDMS-coated coverslip. Upon application of pressure, sealed microreactors are formed and can be imaged from below with a light microscope.

FIG. 17: Schematic depiction of photolithographic fabrication of microreactors in PDMS.

FIG. 18: Microreactors with spatially patterned biotin surfaces. PDMS was patterned with PEG-Biotin and otherwise treated as described in Example 2. Streptavidin-coated 1 micron diameter beads were introduced and bound to the inside of the chambers and not the walls separating the chambers.

FIGS. 19A-B: Demonstration of homogeneous fluorogenic assay for DNA polymerase activity in PDMS microreactors. A) Bright field transmission image (left) of 5 μm diameter microreactors one of which contains a polystyrene bead coated with ˜100 DNA molecules and fluorescence image (right) of the same field-of-view 5 minutes after sealing the poly-C-DNA template-coated bead, φ29 (exo-) DNA polymerase, dGTP-γ-resorufin substrate, and shrimp alkaline phosphatase (SAP). B) Bright field transmission image (left) of 1.5 μm diameter microreactors two of which contain polystyrene beads coated with ˜100 DNA molecules and fluorescence image (right) of the same field-of-view 3 minutes after sealing the poly-C-DNA template-coated beads, Klenow fragment (exo-) DNA polymerase, dGTP-γ-resorufin substrate, and SAP. One of the two microreactors contains more than one bead, and the corresponding fluorescence signal is considerably higher.

FIG. 20: Demonstration of the detection of the signal generated from the incorporation of a single dG4P-3′-O-methyl-fluorescein-5(6)-carboxylic acid substrate from approximately 10,000 DNAs. These DNAs were immobilized on 1 micron streptavidin coated beads that are in turn immobilized in 5 micron microreactors made of PDMS. The image was acquired after 2 minutes of fluorescence signal buildup. Left is the bright field showing the reactors and immobilized beads, and right is the fluorescence image acquired with brightfield fluorescence microscopy. Upon unsealing and resealing the device, no further signal was generated, indicating the reaction has gone to completion.

FIG. 21: Microreactors with spatially patterned biotin surface. PDMS was patterned with PEG-Biotin and otherwise treated as described in Example 5. Streptavidin-coated 1 micron diameter beads were introduced and bound to the inside of the chambers and not the walls separating the chambers.

FIG. 22: 1 micron streptavidin-coated magnetic beads immobilized in microreactors spatially patterned with biotin.

FIG. 23: Images of fluorogenic sequencing according to the invention.

FIG. 24: Images of fluorogenic sequencing of a mixture of nucleic acids according to the invention.

FIG. 25: Fluid handling system for a microfluidic sequencing device. Four pressurized reagent reservoirs, each containing a polymerization reaction mixture for one of four fluorogenic nucleotides along with a wash buffer reservoir, are connected to a manifold of hydraulic valves. Each hydraulic valve is connected to a port on a rotary selector valve which has a single output. The selector valve is motorized and can rotate allowing the selection of a single reagent with minimal mixing and dead volume. The selector valve output is connected to a microfluidic device containing PDMS microreactors. Both the hydraulic valve manifold and the selector valve are computer controlled.

FIG. 26: Fluorescence intensity (after background subtraction) for each sequencing probe cycle corresponding to a microreactor containing a homopolymeric DNA template. The fluorescence intensity was proportional to the length of the homopolymer. Little or no signal was observed in probe cycles that do not correspond to the correct base in the template.

FIG. 27: Fluorescence intensity (after background subtraction) for each sequencing probe cycle corresponding to a microreactor containing a random DNA template. The fluorescence intensity was proportional to the length of homopolymeric sequences in the template. Little or no signal was observed in probe cycles that do not correspond to the correct base in the template.

FIGS. 28A-B: Fluorescence micrographs showing selective patterning of microreactors. A) A micrograph of the reactors focused at a plane level with the opening of the microreactors and B) A micrograph of the deepest part of the microreactors reactors.

FIGS. 29A-B: Fluorescence micrographs showing selective patterning of microreactors with DNA. A) A micrograph of the reactors focused at a plane level with the opening of the microreactors B) A micrograph of the reactors focused at the deepest part of the microreactors.

FIG. 30: Schematic depiction of a device including microreactors for sequencing nucleic acids.

FIGS. 31A-B: A) Fluorescence intensity (after background subtraction) for each sequencing probe cycle corresponding to a microreactor containing a random DNA template and B) calculated sequence based on thresholding of the fluorescence intensity.

FIG. 32: Fabrication of a PDMS microreactor array on a glass coverslip with an ultra-thin PDMS coat using a PDMS micropillar array master.

FIG. 33: Fluorescence image of a fluorophore-filled PDMS microreactor array mounted on a glass coverslip and sealed with a PDMS slab. Many of the fluorophores contained in microreactors in the lower left corner of the array have been photobleached. Because the individual microreactors are sealed, the photobleached region is not replenished by unbleached fluorophores from the other microreactors.

FIGS. 34A-B: Amplification with microreactor PCR. A) Homogeneous end-point fluorescent Taqman signal from PDMS microreactors that were thermocycled with a PCR reaction mixture that did not contain a DNA template. B) Non-uniform end-point fluorescent Taqman signal from PDMS microreactor that were thermocycled with a PCR reaction mixture with a very dilute DNA template sample such that most microreactors would initially contain zero, one, or two template molecules. The bright microreactors contain PCR product.

FIG. 35: Normalized, background-subtracted fluorescence intensity from a single microreactor (top) and base-calling resulting from intensity thresholding (bottom). In both graphs, the black bars are derived from the experimental sequencing data, and the dots represent the theoretical result. In this case, an error-free, 30-base read is obtained from Template A.

FIG. 36: Normalized, background-subtracted fluorescence intensity from a single microreactor (top) and base-calling resulting from intensity thresholding (bottom). In both graphs, the black bars are derived from the experimental sequencing data, and the dots represent the theoretical result. In this case, a 30-base read is obtained from Template B with a single error.

FIG. 37: Normalized, background-subtracted fluorescence intensity from a single microreactor (top) and base-calling resulting from intensity thresholding (bottom). In both graphs, the black bars are derived from the experimental sequencing data, and the dots represent the theoretical result. In this case, an error-free, 39-base read is obtained from Template C.

FIG. 38: Fluorescence image of labeled DNA hybridized to a DNA oligomer that is covalently attached to the inner walls of PDMS microreactors.

FIG. 39A-B: A) Fluorescence image of a labeled-primer that was complementary to a surface-immobilized 5′-benzaldehyde functionalized oligonucleotide that was covalently patterned on the inner walls of PDMS microreactors. B) Fluorescence image of a PDMS microreactor array that was covalently patterned with the same primer as in A), but that was probed with a non-complementary labeled oligonucleotide.

FIG. 40: Fluorescence image of PDMS microreactor array after 10 cycles of TaqMan PCR with rolling circle pre-amplification.

FIG. 41: Schematic of a microfluidic device for on-chip PCR.

FIG. 42: Left: Fluorogenic nucleotide signal generated from immobilized DNA generated from PCR on the walls of a PDMS device. Right: Signal after opening and resealing this device.

### DETAILED DESCRIPTION OF THE INVENTION

We have developed methods and systems for detecting the synthesis of single nucleic acids or an ensemble of substantially identical nucleic acids using fluorogenic nucleotides that are substrates for nucleic acid replicating catalysts and that become able to emit light as a result of incorporation of the nucleotide into a nucleic acid. We have further developed techniques to amplify single molecules of nucleic acids. The invention typically employs microreactors to contain the sequencing or amplification reaction. This invention overcomes limitations of previously proposed techniques.

### Nucleic Acid Sequencing

Advantages of the sequencing methods include:
1) Use of fluorogenic substrates eliminates background from unincorporated labeled nucleotides.
2) Synchronous, ensemble sequencing allows for multiple fields of view to be observed after a single cycle of incorporation, increasing throughput.
3) Large amount of fluorescent product generated allows for simple and economical detection scheme.
4) Allows for a regular, dense array of microreactors enabling high-throughput, parallel nucleic acid sequencing.
5) Reduction in the amount and the cost of reagents (enzyme, labeled nucleotide, nucleic acid, etc.) required for high-throughput sequencing.
6) Phosphate-labeled nucleotides allow for synthesis of natural DNA or RNA, allowing for the sequencing of thousands of nucleotides, in principle.
7) Use of terminal phosphate-labeled nucleotides eliminates the need for chemical modification of DNA following incorporation, decreasing the cycle time.

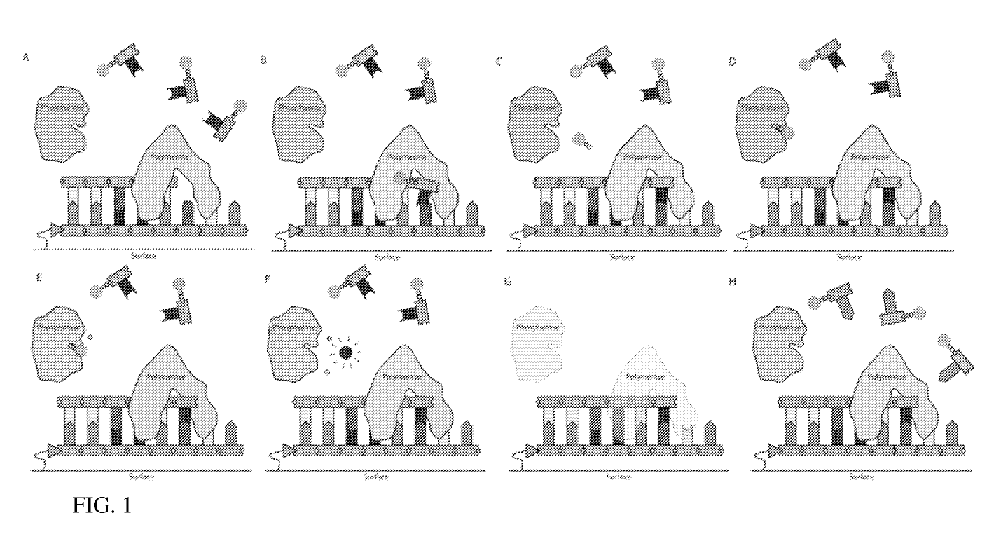

The methods are employed in connection with sequencing by synthesis, in which the incorporation of an individual nucleotide, e.g., including a single base or multiple bases, into a nucleic acid during replication is detected. As nucleotides are incorporated into a nucleic acid that is complementary to the target nucleic acid, the label is rendered able to emit light, e.g., by cleavage from the incorporated nucleotide (e.g., when bound to the terminal phosphate of a nucleotide) (FIG. 1). Preferably, the label is substantially non-emitting when diffusing free in solution to reduce background that could interfere with real time detection of incorporation. Because signal is only generated upon incorporation of the probe nucleotide, the technique distinguishes between incorporation and false binding, i.e., temporary hybridization not resulting in bond formation, and no zero-order waveguide is required. Sequencing may be performed with linear or circular nucleic acids. Sequencing may also be employed isothermally or with thermocycling. Reagents and conditions for amplification, described herein, may also be adapted for sequencing by synthesis.

Incorporation typically results in the cleavage of a portion of the nucleotide, e.g., pyrophosphate, and the label is typically bound to the cleaved portion, i.e., does not form part of the nucleic acid after incorporation. The label may not be immediately fluorescent upon cleavage from the nucleotide. In these embodiments, chemical modification of the label or groups pendant on the label must first occur. For example, certain dyes are non-fluorescent when conjugated to a phosphate group; removal of the phosphate group, e.g., via a phosphatase, then renders the label fluorescent. Other chemical mechanisms that may be involved include acid and base catalyzed reactions and other catalytic processes described herein. Labels may alternatively become able to emit merely as a result of cleavage from the growing nucleic acid. For example, a label may be quenched or otherwise rendered non-emitting by proximity to the nitrogenous base of a nucleotide or a moiety associated with the base.

Preferably, the rate of generation of a fluorophore is more rapid than incorporation of a nucleotide into a nucleic acid. Additionally, any activating catalyst (e.g., alkaline phosphatase) preferably acts rapidly on the fluorogenic label, yielding a fluorophore quickly in comparison to the rate of incorporation.

When each nucleotide is added to the synthesized strand, the nucleotide added is preferably identified. One method of determining the identity of a particular nucleotide is to attach a single label to each nucleotide being added, typically A, T, C, and G, or A, U, C, and G. By sequentially replacing the solution in the microreactor with a solution containing only one of these labeled nucleotide species at a time, microreactors with nucleic acid that is complementary to the added nucleotide species will generate fluorescent label, while other reactors will not. In this manner, the entire sequence of the nucleic acids in all microreactors can be determined.

Because only one labeled nucleotide species is available to the replicating catalyst, e.g., polymerase, at any one time, some catalysts, polymerases, may incorporate the labeled nucleotide species when it is not complementary to the template strand nucleic acid. This misincorporation may remove the nucleic acid strand from subsequent sequencing-by-synthesis cycles, and, over time, reduce the signal generated from each microreactor. To reduce the propensity of the catalyst, e.g., polymerase for misincorporation, non-hydrolyzable nucleotide species may be added to the reaction mixture to compete with the binding of the non-complementary labeled nucleotide species, thereby inhibiting misincorporation. For example, if C is the current base being probed in the microreactor array, the reaction mixture would include fluorogenically labeled dC substrate capable of generating a fluorescent product upon incorporation, as well as non-hydrolyzable nucleotide species that bind to the polymerase in a similar manner to dATP, dTTP, and dGTP. For example, for a dATP analog, dApCpp or dApNHpp might be used, and these non-hydrolyzable dATP structures can serve as examples of other non-hydrolyzable nucleotide analog species by changing the adenosine base moieties to thymine, guanine, uracil, or cytosine. If an activating enzyme is used in the reaction mixture, these non-hydrolyzable nucleotide analogs must be inert to the activities of the activating enzyme. For example, if a phosphatase is used as an activating enzyme, the non-hydrolyzable nucleotide analogs must have their terminal phosphates blocked with, for example, an alkyl group, to eliminate the possibility of a reaction with the phosphatase. Exemplary structures for dNTP analogs are shown below:

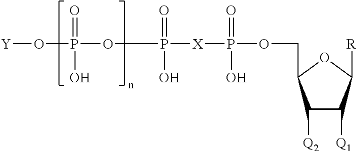

where n=0, 1, 2, 3, or 4, R is a is a nucleoside base, Q₁ and Q₂ are independently hydrogen or hydroxyl, X is a functional group or atom that prevents hydrolysis of the nucleoside analog by a polymerase enzyme, such as methylene or amine, and Y is a substituted or unsubstituted alkyl or aromatic group that prevents digestion of the nucleoside analog by a phosphatase enzyme.

These non-hydrolyzable nucleotide analogs can also be used in conjunction with natural nucleotides to ensure that each cycle of the sequencing reaction reaches completion through the use of a “chase” wash step. For example, after a sequencing cycle that has involved the incorporation of a labeled dATP substrate, non-hydrolyzable nucleotide species that bind to the replicating catalyst, e.g., polymerase, in a similar manner to dCTP, dTTP, and dGTP, along with dATP itself can be introduced to the microreactors. Because the incorporation of labeled nucleotides is typically much slower kinetically than the incorporation of native nucleotides, this chase step will ensure that all appropriate nucleic acid molecules have incorporated dATP and are ready to be probed by the addition of another labeled nucleotide species. The inclusion of non-hydrolyzable nucleotide species that bind to the replicating catalyst, e.g., polymerase, in a similar manner to dCTP, dTTP, and dGTP ensures that the native dATP will not be misincorporated into nucleic acids in which dATP is not complementary to the template strand. If misincorporation is not a significant problem for a specific genus of nucleic acid replicating catalyst, then this chase step can simply include the natural nucleotide analog of the previously used fluorogenic nucleotide analog, allowing for efficient and rapid synchronization of the DNA population.

Sequencing may also be performed using ligase, in which oligonucleotides hybridized adjacent to one another on a template strand are ligated together. Each oligonucleotide employed may be uniquely labeled. Oligonucleotides having the sequence complementary to a region of repeated sequence may be added sequentially using the methods of the invention, and the number of repeats determined by the number of oligonucleotides ligated.

Many proteins and enzymes require metallic co-factors such as divalent metal cations (Mg²⁺, Mn²⁺, Zn²⁺, etc.). For example, magnesium ions may be required for nucleic acid polymerase and alkaline phosphatase activity; manganese ions may be required to enhance the ability of the nucleic acid polymerase to incorporate modified nucleotide substrates (as described in U.S. Pat. No. 7,125,671 and Tabor S., Richardson C. C., Proc. Natl. Acad. Sci. USA, 1989, 86, 4076-4080); and zinc ions may be required for alkaline phosphatase activity. The presence of metal ions at high concentrations can complicate protein-protein interactions, protein-nucleic acid interactions, and surface passivation. In addition, divalent cations can destabilize polyphosphate compounds. Buffer components such as ammonium sulfate and chelating agents can be used to tune intermolecular interactions and control the effective concentration of metal ions. Many nucleic acid polymerizing replicating catalysts also require a reducing environment to perform optimally. There are many classes of reducing agents such as thiols (such as 2-mercaptoethanol or dithiothreitol) and phosphines (such as tris(2-carboxyethyl)phosphine (TCEP)), which are compatible with physiological buffers.

An individual sequencing reaction may be controlled by the introduction of Mg or Mn ions, nucleotides, and other co-factors necessary to effect replication. Other methods for controlling replication include changing the temperature or introducing or removing substances that promote or discourage complex formation between the target and catalyst. The catalyst or target may also be rendered inoperative to end sequencing, e.g., through denaturation or cleavage.

Multiplexing, i.e., detection of more than one replication at a time, may also be employed to increase throughput.

### Fluorogenic Labels

Any label that becomes able to emit light as a result of incorporation of a nucleotide to a synthesized nucleic acid may be employed in the methods of the invention. Labels can be attached to nucleotides at a variety of locations. Attachment can be made either with or without a bridging linker to the nucleotide. The label may be attached to the base, sugar, or phosphate of the nucleotide. Preferably, the label is attached to the terminal phosphate, so it is cleaved from the nucleotide during replication. Labels may also be attached to non-naturally occurring portions of a nucleotide, e.g., to the delta or epsilon phosphate in a tetra- or pentaphosphate containing nucleotide. Alternatively, labels may be attached to the alpha phosphate and displaced during incorporation of a nucleotide in a synthesized strand. For clarity, fluorogenic labels, as employed in the invention, do not include fluorophore-quencher pairs, in which a quenching moiety appended to a nucleotide prevents fluorescence by resonance energy transfer from the fluorophore. Some quenching by the base, sugar, or phosphate in a nucleotide may occur with a fluorogenic label.

In certain embodiments, the label is destroyed (or rendered non detectable) once detected. One method for destroying the label is photobleaching. Another method is to wash out this label by opening the microreactors and allowing buffer exchange through fluid flow and diffusion.

Bulk nucleic acid sequencing reactions rely upon enzymatic amplification of nucleic acid molecules to generate large numbers of fluorescently labeled molecules for each sequenced base. The large number of labels detected relaxes constraints on the chemical stability, photostability, brightness, and protein-dye interactions, as well as spectral separation between different labels.

Nucleic acid sequencing reactions also typically occur in a narrow range of conditions in which the replicating catalyst, e.g., polymerase, and associated enzymes (such as alkaline phosphatase) operate optimally. These conditions vary considerably depending on the particular enzymes involved. One critical parameter with respect to fluorogenic label selection is the pH under which the sequencing reaction will take place (typically within the physiological pH range of 6 to 9), because the absorption and emission spectra of the product fluorophores are often strongly pH-dependent. For example, it is desirable for fluorogenic substrates that produce phenolic fluorophores to have pKₐ's below 7.

Below we list preferred criteria for fluorogenic labels for use in high-fidelity, fluorogenic sequencing:

1) No reactivity or detrimental interaction with buffer components, enzymes, nucleic acids, or other dyes or substrates.

Sequencing can involve a complicated set of proteins including nucleic acid replicating enzymes, activating enzymes to digest fluorogenic substrates resulting from the incorporation of labeled nucleotides (such as alkaline phosphatase), blocking proteins for surface passivation, and oxygen scavenger enzymes for mitigating photodamage. Nonspecific interactions between fluorogenic substrates/fluorophores with proteins can result in quenching via electron transfer, energy transfer, or chemical reactions that result in spectrally modified fluorophores. Such interactions can compromise nucleic acid sequencing by damaging the substrate, reducing fluorescence emission, or altering protein function. For example, many fluorophores have complicated interactions with reducing agents. In addition, proteins commonly have solvent exposed residues containing thiol moieties. The ground and excited states of several commonly used fluorogenic dyes such as resorufin and 7-hydroxy-9H-(1,3-dichloro-9,9-dimethylacridin-2-one) (DDAO) are susceptible to nucleophilic attack by thiols. Fluorescein analogs with certain patterns of halogenation are similarly vulnerable. Fluorogenic substrates may also be susceptible to nucleophilic attack by buffer components, despite the resistance of the corresponding fluorescent product. Fluorogenic substrates and fluorophores that react and interact minimally with the components of the sequence reaction are preferred for fluorogenic sequencing. Chemical modification can be rationally employed on the fluorogenic labels/fluorophores to impart resistance to these effects (see, e.g., U.S. Pat. Nos. 7,432,372, 6,162,931, and 6,229,055 and WO 2005/108994 A1).

2) Fluorogenic labels are preferably resistant to photodamage and preferably do not emit significantly in the detection band(s).

To maximize signal to noise of the method, fluorogenic molecules within the detection volume are preferably substantially non-fluorescent when exposed to the excitation wavelengths. Preferably, these fluorogenic molecules have a very small extinction coefficient at these excitation wavelengths, such that they do not absorb photons when excited. Alternately, the fluorogenic molecules may have measurable absorbance at the excitation wavelengths of the fluorescent label, but thermal relaxation is the dominant process moving the substrate from the excited state to the ground state, substantially eliminating the possibility of fluorescence emission. In another embodiment, the substrate may absorb appreciably at the excitation wavelengths of the fluorescent label but emit fluorescence that is spectrally separated from the fluorescence generated by the fluorescent label. It is preferable for the fluorogenic substrate not to absorb the excitation light significantly, to limit time spent in the excited state, reducing the potential for any excited-state chemistry or bleaching.

3) Preferably, fluorophores produce a high photon flux at visible wavelengths. Preferred fluorescent labels generate large photon fluxes (with high quantum efficiency) at wavelengths well-separated from the excitation wavelength and bleach into breakdown products that are substantially unreactive. In order to increase signal, triplet state quenchers, such as those described in US 2007/0161017 A1, may be used.

The presence of molecular oxygen in the reaction chamber can also bleach fluorophores, reducing the average total number of photons generated during detection. A variety of methods for eliminating molecular oxygen from a reaction sample (including enzymatic systems of catalase and glucose oxidase or protocatechuate 3,4-dioxygenase) are known in the art (see, e.g., US 2007/0161017 A1).

Transient interactions with a surface (e.g. the surface of the microreactor) or buffer components, such as proteins at high concentration in the sequencing mixture, may quench fluorescence, creating spurious signal variations. Because high protein concentration in solution can cause nonspecific quenching of fluorescence, an example of a protein-free system for reducing nonspecific adsorption to surfaces is also described herein.

Exemplary labels include resorufin and 91′-(1,3-dichloro-9,9-dimethylacridin-2-one) (DDAO). Additional labels are known in the art, e.g., in U.S. Pat. Nos. 7,041,812, 7,052,839, 7,125,671, 7,223,541, and 7,244,566.

Previous embodiments of fluorogenic nucleic acid sequencing have relied on a relatively narrow class of fluorogenic dyes for labeling nucleotide substrates (e.g., U.S. 2004/015119 and U.S. Pat. No. 7,125,671). In particular, phenolic dyes such as fluoresceins, phenoxazines (such as resorufin), acridines (such as DDAO), and coumarins may be used in fluorogenic substrates. The chemistry of fluorogenic nucleic acid substrates based on phenolic dyes is relatively straightforward because the phenolic oxygen is esterified to a phosphate group. This substrate chemistry excludes the use of other potentially useful fluorogenic dyes such as those containing amines (e.g., rhodamine and its derivatives, cresyl violet, etc.). Once a DNA polymerase incorporates a labeled dNTP, cleaving between the α- and β-phosphates of the nucleotide, the liberated fluorophore becomes fluorescent, either directly upon cleavage from the dNTP, or after further enzymatic action of other enzymes (Sood et al. J. Am. Chem. Soc., 2005, 127, 2394-2395 and Kumar et al. Nucleotides, Nucleosides, and Nucleic Acids, 2005, 24, 401-408) (through a coupled enzyme assay discussed further below). These newly fluorescent molecules are then detected using standard fluorescence detection techniques (English et al. Nat. Chem. Biol., 2006, 2, 87-946) (such as total internal reflection fluorescence, epifluorescence, or confocal microscopy).

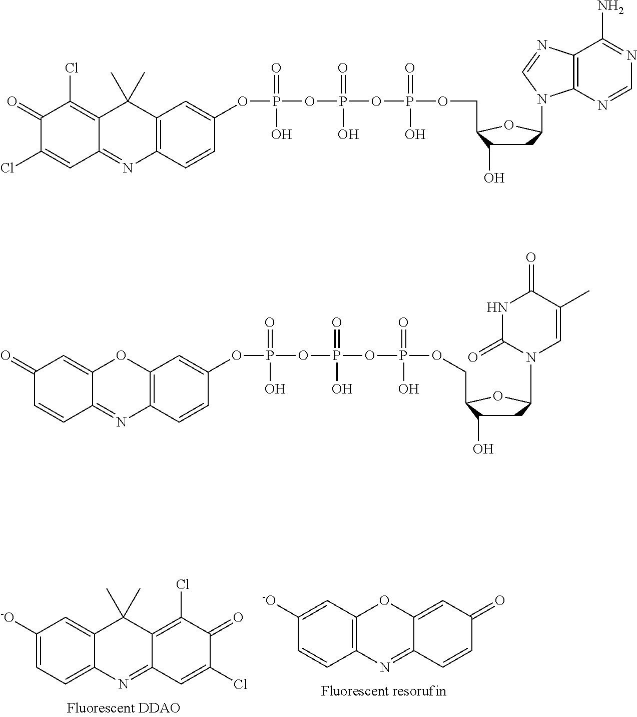

Resorufin is not fluorescent when conjugated to dNTPs, while for DDAO the fluorescence and absorption spectra change significantly when it is conjugated to dNTPs. Upon cleavage from the dNTP, e.g., through the action of DNA polymerase, these molecules still have phosphate groups covalently linked to the fluorophore, which must be removed before the molecule becomes fluorescent.

Additional labeled nucleotides employ a fluorescein-based fluorophore:

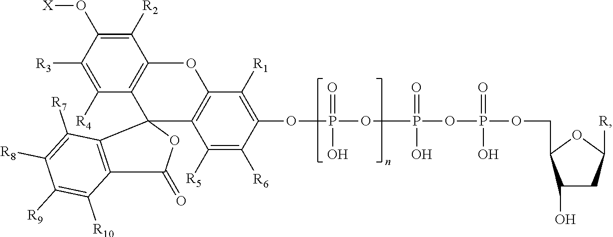

where R is a nucleoside base, as described herein, n is 0 to 4, and X is a blocking group that serves to minimize the fluorescence emission of the substrate molecule. This blocking group is, for example, an alkyl group (e.g., such as methyl, ethyl, propyl, isopropyl, and butyl), an acyl group (e.g., acetyl), an amide group (e.g., C(O)NR_{A}R_{B}, where R_{A} and R_{B} are independently C₁-C₆ alkyl or R_{A} and R_{B} together for a 3-8-membered heterocycle, optionally containing additional nitrogen, oxygen, or sulfur atoms, e.g., morpholine), sulfonyl (e.g., SO₂R, where R is C₁-C₆ alkyl), an alkyl group interrupted with one or more heteroatoms (e.g., O, N, S, or P), haloalkyl group (e.g., perfluorinated alkyl), cycloalkyl (e.g., with 3-6 ring carbons), carboxy substituted alkyl, sulfonyl substituted alkyl, or any other functional group that prevents the electronic structure of the attached oxygen from imparting significant fluorescence to the substrate molecule (see, e.g., WO 2005/108994). The functional groups R₁-R₁₀ are chosen to enhance the properties of the fluorogenic substrate and corresponding fluorophore to satisfy the requirements for nucleic acid sequencing described above. These groups may be selected from hydrogen, halogen (e.g., F or Cl), sulfonate (i.e., SO₃H), carboxy, acyl, alkyl, alkoxy, alkylthio, aryl, heteroaryl (e.g., containing one or more O, N, or S), nitro, and hydroxyl (see also U.S. Pat. Nos. 7,432,372, 6,162,931, and 6,229,055 and WO 2005/108994 A1). Particular examples of fluorogenic nucleotide substrates with these modifications are as follows. Structures of fluorescein-based fluorogenic nucleotide substrates for fluorogenic nucleic acid sequencing where R is a nucleotide base, n is 0 to 4, and X is a blocking group designed to minimize the absorption and fluorescence emission of the fluorogenic substrate, and n is an integer between 0 and 4. A) Substrate based on 6-carboxyfluorescein (6-FAM). B) Substrate based on 6-carboxyhexachlorofluorescein (6-HEX). C) Substrate based on 6-carboxytetrachlorofluorescein (6-TET). D) Substrate based on 6-carboxy-4′,5′-dichloro-2′,7′-dimethoxyfluorescein (6-JOE). E) Substrate based on Oregon Green™ 488. F) Substrate based on Oregon Green™ 514. G) Substrate based on 2,7-dichlorofluorescein.

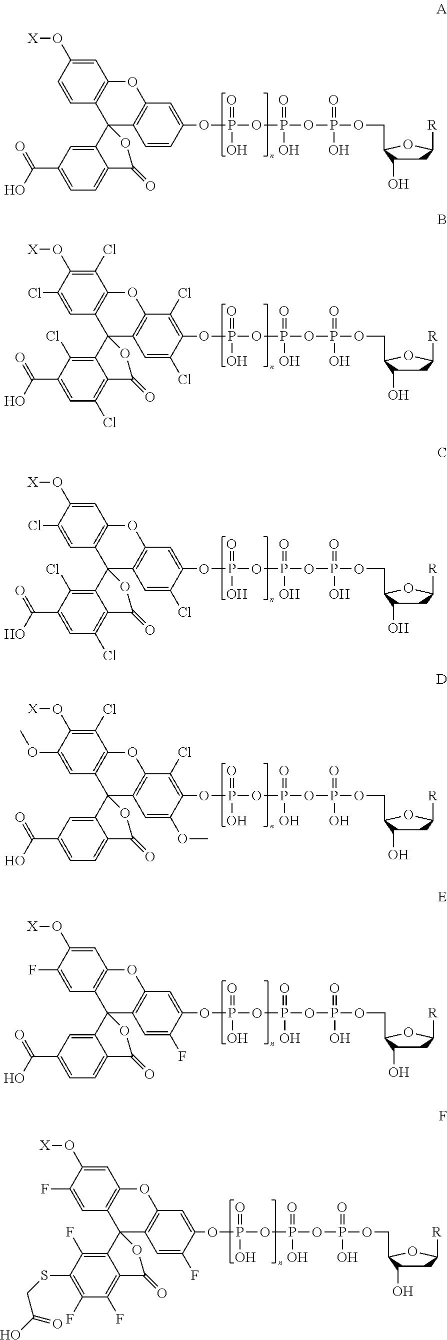

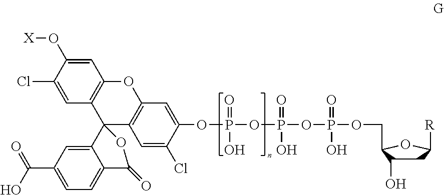

Another class of fluorogenic substrates has the general formula:

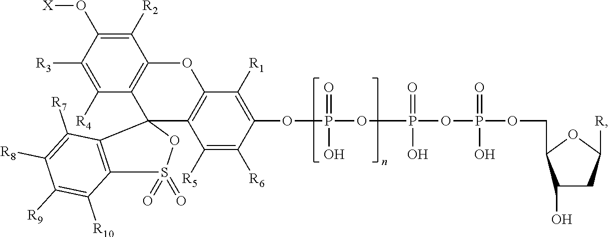

with R, X, and R₁-R₁₀ as described above. The fluorogenic dyes used in these substrates can be synthesized using methods known in the art (U.S. Pat. No. 6,130,101, U.S. 2005/0026235, and Pongev et al., Rus. J. Gen. Chem, 2001), and the corresponding substrates can be generated using the procedure described in WO 2010/017487.

A third class of fluorogenic compounds has the following structure:

Base-Sugar-Phosphate-[Self-reacting Component],

where Base is any nucleotide base as described herein, Sugar is any sugar or other such group in a nucleotide as described herein, Phosphate is a polyphosphate, and Self-reacting Component is a moiety that undergoes an intramolecular reaction upon cleavage of the phosphate to which it is connected to form a fluorophore. These compounds are substantially non-fluorescent at the wavelengths where the corresponding fluorophore emits and typically absorb very little at the absorption maximum of the corresponding fluorophore. The Self-reacting Component is of two forms. In one, this component includes a self-immolative linker conjugated to a fluorophore, wherein the conjugation renders the fluorophore substantially non-fluorescent. When the phosphate group is cleaved from the self-immolative linker, it spontaneously reacts, resulting in release of the fluorophore, which is fluorescent again. In another form, this component includes a proto-fluorophore, which is substantially nonfluorescent. Cleavage of the phosphate group from the proto-fluorophore results in an intramolecular reaction, e.g., lactonization, that forms a fluorophore. It will be understood that the compounds depicted above will be linked as is known in the art to produce a nucleotide, as defined herein, having a fluorogenic label.

An example of a fluorogenic substrate having a self-immolative linker is as follows:

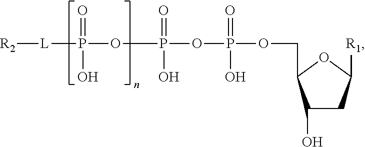

where R₁ is a nucleotide base, L is a self-immolative linker, n is an integer ranging from 0 to 4, and R₂ is a fluorogenic moiety.

Self-immolative linkers are known in the art (see, e.g., Zhou et al., ChemBioChem, 2008, 9, 714-718; Levine et al., Molecules, 2008, 13, 204-211; Lavis et al., ChemBioChem, 2006, 7, 1151-1154; Richard et al., Bioconjugate Chemistry, 2008, 19, 1707-1718; U.S. 2005/0147997; and U.S. 2006/0003383). An example of a self-immolative linker is the trimethyl lock linker (Levine et al., Molecules, 2008, 13, 204-211 and Lavis et al., ChemBioChem, 2006, 7, 1151-1154):

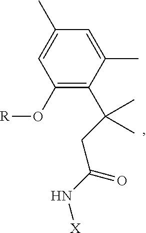

where R is an enzyme substrate moiety (e.g., phosphate), and X—NH₂ is a fluorophore. A fluorogenic nucleotide substrate having the trimethyl lock has the general structure:

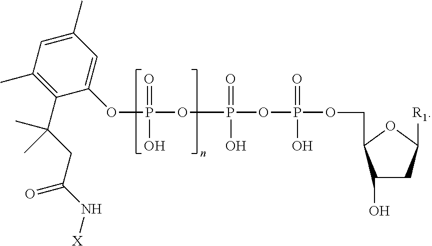

One class of amine-containing fluorophores includes rhodamine derivatives, where the corresponding nucleotide substrate has the general structure:

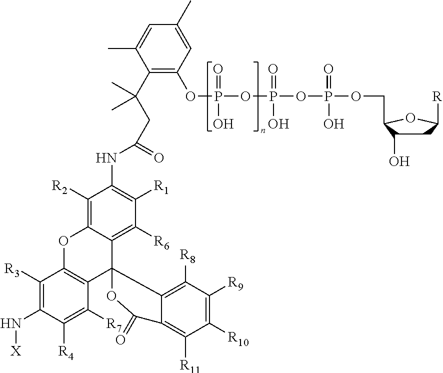

where R is a nucleotide base, n is an integer ranging from 0 to 4, and X is a blocking group (as discussed above, e.g., C(O)-morpholinyl) that serves to minimize the fluorescence emission of the chromophore when it is conjugated to the substrate. The groups R₁-R₄ and R₆-R₁₁ are all hydrogen atoms in the case of rhodamine but can be modified to form derivatives with different chemical, spectral, and photophysical properties. R₁-R₄ and R₆-R₁₁ can be hydrogen, halogen (e.g., F or Cl), sulfonate, carboxy, acyl, alkyl, alkoxy, alkylthio, aryl, heteroaryl (e.g., containing one of O, N, or S), nitro, or hydroxyl, which may be substituted as described herein. Exemplary rhodamine dyes include rhodamine B, rhodamine 19, rhodamine 110, rhodamine 116, sulforhodamine B, and carboxyrhodamine.

Derivatives of oxazine dyes can also be employed in a similar fashion:

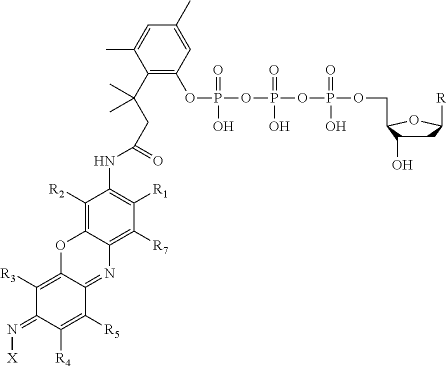

where R is a nucleoside base, n is an integer between 0 and 4, X is a blocking group (as discussed above) that serves to minimize the fluorescence emission of the chromophore when it is conjugated to the substrate, and R₁-R₅ and R₇ represent functional groups as discussed for rhodamine. An exemplary oxazine dye is 3-imino-3H-phenoxazin-7-amine (oxazine).

Benzophenoxazine dyes, such as cresyl violet and its derivates, can also be employed:

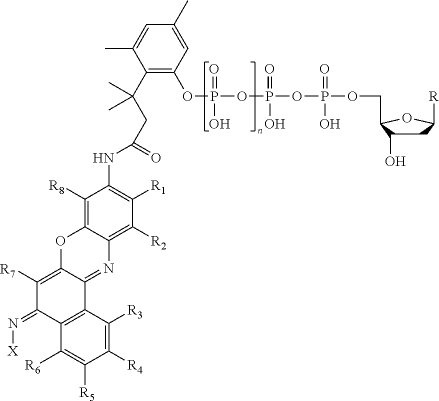

where R is a nucleoside base, n is an integer between 0 and 4, X is a blocking group (as discussed above) that serves to minimize the fluorescence emission of the chromophore when it is conjugated to the substrate, and R₁-R₈ represent the functional groups as discussed for rhodamine. An example of a benzophenoxazine dye is 9-imino-9H-benzo[a]phenoxazine-5-amine.

These compounds will be incorporated by a nucleic acid replicating catalyst into a nucleic acid and yield a polyphosphate chain terminated by the self-immolative linker conjugated to the fluorophore:

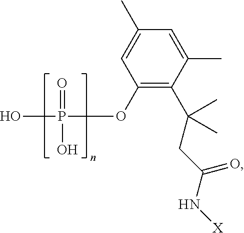

where X—NH₂ is a fluorophore. A phosphatase can then be used to cleave the polyphosphate chain leading to the generation of the following species:

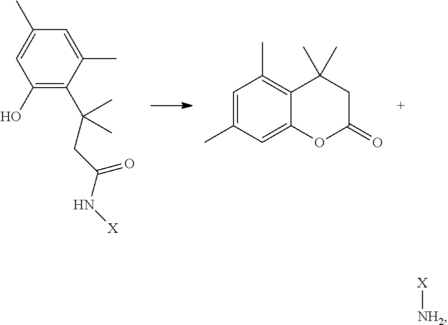

resulting in the generation of an amine-containing fluorophore.

The Self-reacting Component may also result in spontaneous generation of a fluorophore, e.g., through cyclization reactions in response to enzymatic digestion. Fluorogenic nucleotide substrates based on self-generating fluorophores with the general structure given below can be used for nucleic acid sequencing:

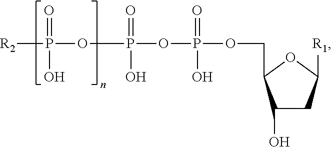

where R₁ is a nucleotide base, n is an integer between 0 and 4, and R₂ is a moiety that undergoes an intramolecular reaction to form a fluorophore upon removal of the phosphate. An example of these compounds results in generation of a coumarin fluorophore (see, e.g., Wang et al., Methods in Molecular Medicine, 1998, 23, 71; Wang et al., Bioorganic and Medicinal Chemistry Letters, 1996, 6, 945-950; and U.S. Pat. No. 6,214,330):

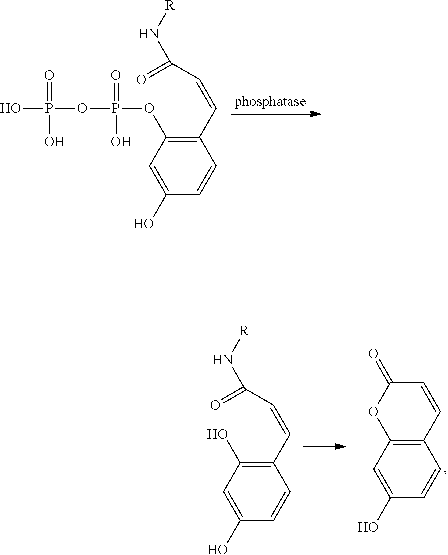

where R represents any suitable substituent for the amine leaving group. Examples of structures of coumarin-generating fluorogenic nucleotide substrates for fluorogenic nucleic acid sequencing where R₁ is a nucleotide base are A) substrate based on 7-hydroxycoumarin; B) substrate based on coumarin 102; C) substrate based on 6,8-difluoroumbelliferone; and D) substrate based on coumarin.

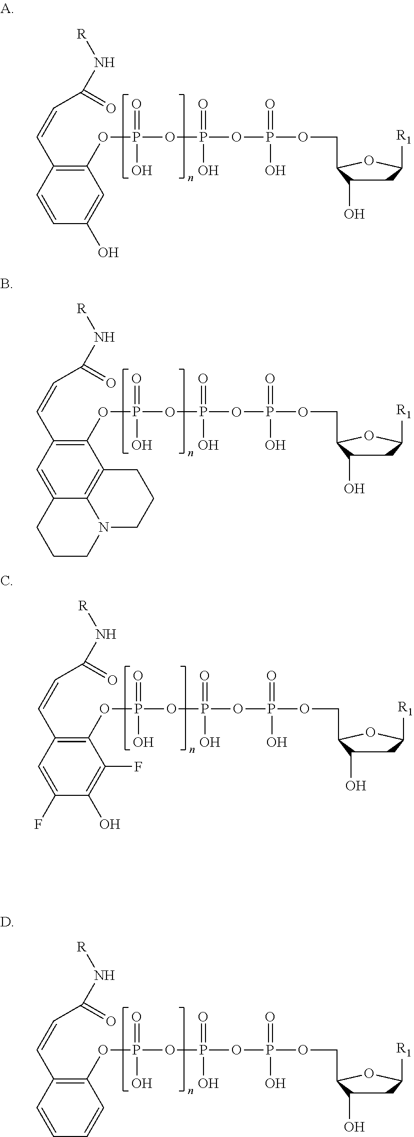

Additional fluorogenic nucleotide substrates are described in U.S. 2010/0036110 and WO 2010/017487, both of which are incorporated by reference. It will also be understood that the sugar moiety depicted in any of the above structures, i.e., 2′-deoxyribose, may be replaced with any other appropriate group, as described herein (for example, the nucleotide may be a ribonucleotide).

### Microreactors

Massively parallel nucleic acid sequencing requires a method of capturing, spatially arranging, and, in most cases, amplifying a target nucleic acid sample for sequencing. The microreactor array offers not only a reaction confinement method for fluorogenic sequencing but also a natural platform for nucleic acid capture and amplification. Accordingly, the reagents for sequencing and/or amplification of nucleic acids are disposed in a microreactor. Exemplary microreactors hold volumes of 0.0001 fL, to 100000 fL, although larger volumes are possible. Conducting fluorogenic sequencing and/or amplification in a microreactor imparts several advantages as described herein. A single microreactor may be employed, or a device having numerous microreactors may be employed, e.g., a solid substrate having 10, 50, 100, 500, or more microreactors arranged as desired, e.g., an ordered array.

For sequencing, an ensemble of identical nucleic acids (generally clonally amplified from a single nucleic acid) is immobilized in each microreactor. The activating catalyst, or replicating catalyst may also be immobilized within the microreactor. Methods for immobilizing nucleic acids or catalysts are well known in the art and include biotin-streptavidin, antibody-antigen interactions, covalent attachment, or attachment to complementary nucleic acid sequences.

A target nucleic acid, activating catalyst, or replicating catalyst may be immobilized to beads (magnetic, paramagnetic, polystyrene, glass, etc.) using immobilization techniques well known in the art. When the nucleic acid is immobilized to a bead, these beads can then be trapped in microreactors, and the nucleic acid can be directly amplified or sequenced according to the invention. Affinity capture beads may also be used to capture relevant nucleic acids, e.g., eukaryotic RNA can be specifically extracted by annealing poly-dT coated beads to the poly-A tail of the mRNAs.

In order to trap a population of substantially identical nucleic acids within a microreactor, spatial patterning of the microreactor with non-covalent or covalent reactive groups may be employed so that nucleic acid binds only to the interior of the microreactor.

Materials that are useful in forming the microreactors include glass, glass with surface modifications, silicon, metals, semiconductors, high refractive index dielectrics, crystals, gels, lipids, and polymers (e.g., poly(dimethylsiloxane) (PDMS)). Mixtures of materials may also be employed.

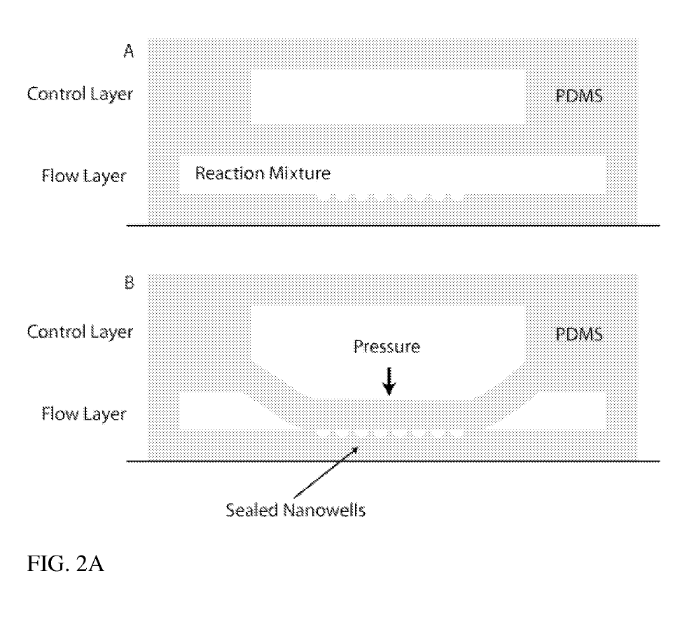

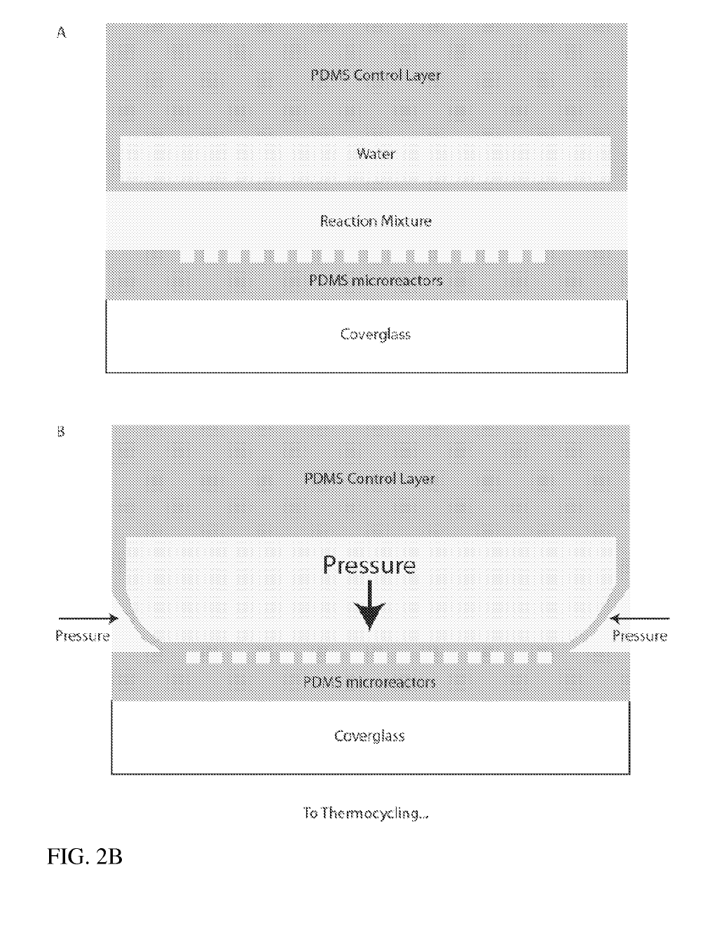

An exemplary method of fabricating microreactors in PDMS is described herein (FIG. 2). Other materials for microreactor fabrication include polytetrafluoroethylene, perfluoropolyethers, and parylene. Additionally, lipid vesicles can be generated using standard lipid extrusion techniques (Okumus et al. Biophys. J. 2004, 87(4), 2798-2806) and used to confine the reaction. Another method of generating microreactors is the creation of an emulsion of the reaction mixture in an immiscible solvent such as mineral oil or silicon oil. These and other methods for manufacturing microreactors are known in the art, e.g., U.S. Pat. Nos. 7,081,269, 6,225,109, 6,225,109, and 6,585,939.

An ensemble of substantially identical target nucleic acids (or replicating catalyst) can be delivered to a microreactor using methods known in the art. One method employs emulsion PCR to generate a population or colony of substantially identical nucleic acids on a bead (Dressman (2003) Proc. Natl. Acad. Sci. USA 100:8817; Brenner et al. (2000) Nat. Biotech. 18:630). Another method for delivery is to provide a dilute solution of nucleic acid so that each microreactor, on average, holds fewer than one molecule. Using this approach some microreactors will have no target nucleic acid, some will have a single target nucleic acid, and a very small number will have more than one. As further described herein, single molecules of nucleic acid can be delivered to microreactors via beads. Then solid-phase PCR, rolling circle amplification, or other amplification technique, can be conducted on these immobilized single molecules, building up a population or colony of substantially identical nucleic acids. When employing beads, amplification may occur with or without the bead in the microreactor. Fluorophores and fluorogenic labels are preferably trapped in the microreactor during the course of a sequencing run. If either the generated fluorophore or the fluorogenic-label escapes the reactor, then information regarding the sequencing of the nucleic acid may be lost. Materials and methods for retaining fluorophores and fluorogenic substrates within a reactor are described herein.

Microreactors are preferably manufactured from materials that prevent or reduce diffusion of fluorophores, evaporation of water, and nonspecific absorption of proteins. Alternatively, microreactors are treated to prevent or reduce such diffusion, evaporation, and nonspecific absorption. Treatment methods are described herein.

Microreactors may or may not have lids to enclose the reaction mixture. When a lid is employed, the nucleic acid may be immobilized on it. The lid can be sealed by conformal pressure, adhesives, and other bonding techniques known in the art. An exemplary process for sealing microreactors made from PDMS (or other elastomeric materials) is shown in FIGS. 2A-2B. This process employs valve technology known in the art (Unger, M. A. et al. 2000. Science, 288, 113-116; Jung et al. Langmuir, 2008. 24, 4439-4442). Lids made from glass and other optical quality materials are preferred.

An alternative sealing method employs a fluid immiscible with aqueous solutions, e.g., an oil. For example, oil can be applied uniformly over an array of microreactors, resulting in high fidelity seal. In addition, oils may enhance the thermal stability of small volumes of aqueous solution, preventing evaporation during thermocycle sequencing or PCR. Examples of such oils are mineral oil, silicon oils (such Ar20 silicone oil), fluorinated oils (such as perfluorocarbons and HFE-7500, 2-trifluoromethyl-3-ethoxydodecafluorohexane, or Fluorinert), or hydrocarbon oils (such as isoparaffinic hydrocarbons, e.g., Isopar M). These oils may also contain surfactants to alter their material properties. Examples of such surfactants include Span 80, Tween-20, Tween-80, Triton X-100, ABIL EM90, ABIL WE 09, Tegosoft Liquid, Sun Soft, Lubrizol U, PEG-perfluoropolyethers, Pluronic-F108, ethylenediamine tetrakis(ethoxylate-block-propoxylate) tetrol (Tetronic), and DC 749. Other oils and surfactants are known in the art.

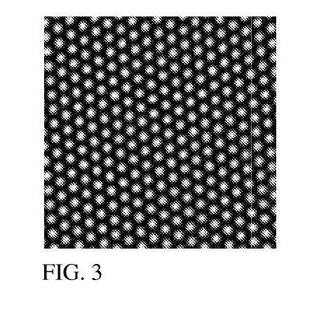

In one embodiment, PDMS microreactors are sealed with a viscous oil by first introducing a desired aqueous solution to the microreactors and then rapidly flowing in a viscous oil, typically neat, to cover the top of the microreactors and prevent diffusion or evaporation of components of the solution. This seal is demonstrated in FIG. 3, where an aqueous solution of carboxyfluorescein (10 μM) is introduced to the microreactors. Silicone oil (Sigma) is then passed over the microreactor array, covering the tops of the individual microreactors and preventing diffusion of the fluorophore or evaporation of the solvent. This sealing technique can also be applied to other types of microreactor arrays, e.g., glass or UV fused-silica.

### Activating Catalyst

Any catalyst that is capable of acting on a label to render it fluorescent after a nucleotide incorporation event may be used in the invention. Preferably, the activating catalyst does not act on the label prior to incorporation. Preferred catalysts include enzymes such as alkaline phosphatases (e.g., bacterial alkaline phosphatase, shrimp alkaline phosphatase, calf intestinal phosphatase, and antarctic phosphatase), acid phosphatases, galactosidases, horseradish peroxidase, phosphodiesterase, phosphotriesterase, pyruvate kinase, lactic dehydrogenase, lipase, or combinations of enzymes and substrates in a coupled enzyme system such as maltose, maltose phosphorylase, glucose oxidase, horseradish peroxidase, and amplex red (PIPER™ phosphate detection kit, Invitrogen). The activating catalyst may also be an ion in solution, e.g., iodide, hydroxide, or hydronium, a zeolite or other porous catalytic surface, or a metal surface, e.g., platinum, palladium, or molybdenate. Other biological and synthetic catalysts may also be employed. Multiple copies of a particular catalyst may be present to reduce the time required for interaction with the label. The catalyst may be immobilized to a surface of the microreactor or a bead to increase the effective concentration within the reactor.

### Nucleic Acids and Nucleotides

The invention may be employed with any nucleic acid (e.g., DNA, RNA, and DNA/RNA) using any appropriate nucleic acid replicating catalyst. Nucleotides may be naturally occurring or synthetic, e.g., synthetic ribonucleosidyl, 2′-deoxyribonucleosidyl, Locked Nucleic Acid, peptide nucleic acid, glycerol nucleic acid, morpholino nucleic acid, or threose nucleic acid connected, e.g., via the 5′, 3′, or 2′ carbon of the radical, to a phosphate group and a base. The nucleotide may include a purine or pyrimidine base, e.g., cytosine, guanine, adenine, thymine, uracil, xanthine, hypoxanthine, inosine, orotate, thioinosine, thiouracil, pseudouracil, 5,6-dihydrouracil, and 5-bromouracil. The purine or pyrimidine may be substituted as is known in the art, e.g., with halogen (i.e., fluoro, bromo, chloro, or iodo), alkyl (e.g., methyl, ethyl, or propyl), acyl (e.g., acetyl), or amine or hydroxyl protecting groups. In certain embodiments, the nucleotides employed are dATP, dCTP, dGTP, and dTTP. In other embodiments, the nucleotides employed are ATP, CTP, GTP, and UTP. Ribosides may be employed for sequencing DNA, e.g., when DNA-dependent RNA polymerase is employed. Ribosides may be employed for sequencing RNA, e.g., when RNA-dependent RNA polymerase is employed. Deoxyribosides may also be employed for sequencing RNA, e.g., when reverse transcriptase is employed. In preferred embodiments, the sequencing methods of the invention produce a nucleic acid that is complementary to the target nucleic acid and that includes only naturally occurring nucleotides, i.e., the label is removed during incorporation. Alternatively, nucleotides may include a moiety that is retained in the synthesized nucleic acid. Such moieties are preferably present on fewer than all of the labeled nucleotides employed, e.g., only one, two, or three, to minimize disruption of replicating catalyst activity.

### Nucleic Acid Replicating Catalysts

Exemplary replicating catalysts include DNA polymerases, RNA polymerases, reverse transcriptases, ligases, and RNA-dependent RNA polymerases. Exemplary DNA polymerases include E. coli DNA polymerase I, E. coli DNA polymerase I Large Fragment (Klenow fragment), Klenow fragment (exo-), Sequenase™, phage T7 DNA polymerase, T4 DNA polymerase, Phi-29 DNA polymerase, Phi-29 (exo-) DNA polymerase, Bsu DNA polymerase (exo-), thermophilic polymerases (e.g., Thermus aquaticus (Taq) DNA polymerase, Thermus flavus (Tfl) DNA polymerase, Thermus thermophilus (Tth) DNA polymerase, Thermococcus litoralis (Tli) DNA polymerase, Pyrococcus furiosus (Pfu) DNA polymerase, Vent™ DNA polymerase, or Bacillus stearothermophilus (Bst) DNA polymerase, Therminator™, Therminator II™, Therminator III™, and Therminator-γ™), Vent™ (exo-) DNA polymerase, Deep Vent™ (exo-) DNA polymerase and reverse transcriptase (e.g., AMV reverse transcriptase, MMLV reverse transcriptase, SuperScript-I™, SuperScript-2™, SuperScript-3™, or HIV-1 reverse transcriptase). In addition, existing polymerase enzymes can be rationally mutated or selected using directed evolution to enhance the efficiency and fidelity with which they incorporate modified nucleotides (U.S. 2007/0196846, U.S. 2007/0172861, and U.S. 2007/0048748). Other suitable DNA polymerases are known in the art. Exemplary RNA polymerases include T7 RNA polymerase, T3 RNA polymerase, SP6 RNA polymerase, and E. coli RNA polymerases. Exemplary ligases are known in the art. Exemplary RNA-dependent RNA polymerases are known in the art. Catalysts may bind to a target at any appropriate site as is known in the art.

Multiple copies of the replicating catalyst are preferentially present. If a particular catalyst molecule disassociates from the template strand, another catalyst molecule may bind and continue replication without affecting the sequencing function.

### Reversible Terminators

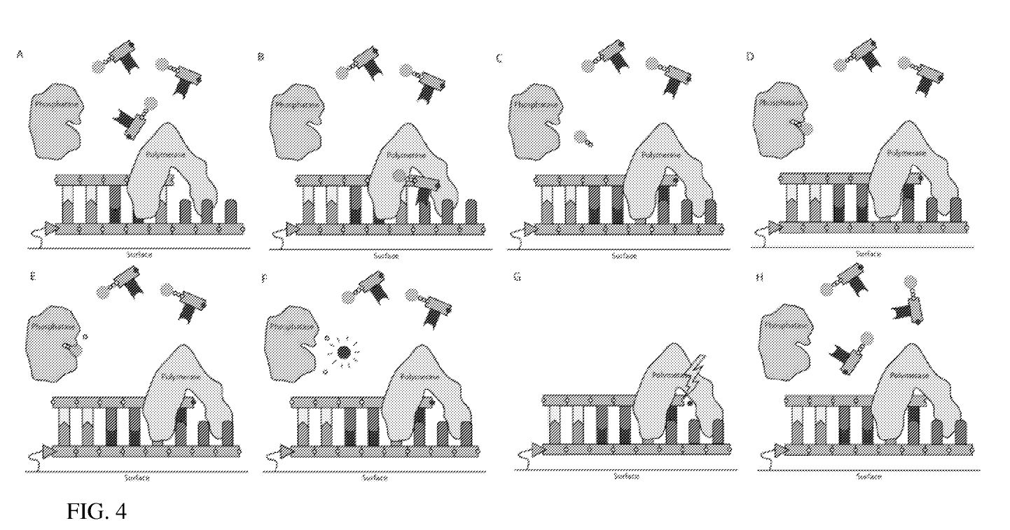

In the case of a homopolymer sequence, multiple fluorogenic nucleotide substrates will be incorporated by the polymerase enzyme resulting in the generation of more fluorescent product than in the incorporation of a single base. In principle, the amount of signal generated by primer extension with a homopolymeric template is proportional to the number of consecutively repeated bases in the homopolymeric template. However, kinetic complications can arise because the amount of time required to complete primer extension with a homopolymeric template is considerably longer than that required for a single base incorporation. In these circumstances, reversible terminator nucleotide analogs may be employed. Terminator nucleotides only allow the incorporation of a single base by a replicating catalyst, e.g., polymerase enzyme, because they possess a protecting group on the 3′-hydroxyl of the sugar moiety, which prevents subsequent primer extension, even in the case of a homopolymeric template region. Reversible terminator nucleotides are terminator nucleotides where the protecting chemistry on the 3′-hydroxyl group can be reversed in a controlled way, allowing primer extension to occur at a later time. Hence, one can employ reversible terminators in synchronous sequencing-by-synthesis in order to avoid deletion errors in the sequencing of homopolymeric regions by adding an extra step in each cycle to reverse the terminating protection chemistry (see FIG. 4)

Fluorogenic reversible terminator nucleotides provide the advantages of synchronous fluorogenic sequencing-by-synthesis along with the additional benefit of facile homopolymer sequencing. A general structure of a fluorogenic reversible terminator nucleotide is given below:

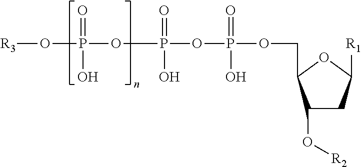

where R₁ is a nucleoside base, R₂ is a reversible terminator, R₃ is a fluorogenic dye, and n is an integer between 0 and 4. An exemplary reversible terminator protecting group is the 3′-O-azidomethyl moiety which can be converted into a 3′-hydroxyl by the addition of tris(2-carboxyethyl)phosphine (TCEP). An example of a fluorogenic reversible terminator nucleotide, 3′-O-azidomethyl-2′-deoxythymine-tetraphosphate-6-3-O′-methylcarboxyfluorescein is given below:

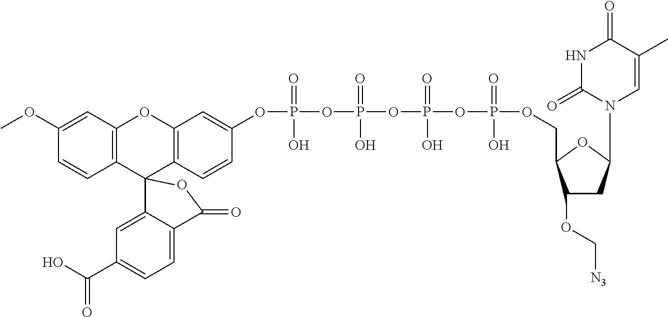

In this case, a replicating catalyst, e.g., polymerase enzyme, would incorporate the above substrate into a template nucleic acid resulting in the generation of 3-O′-methylcarboxyfluorescein triphosphate (which would be digested by alkaline phosphatase to the fluorescent product molecule 3-O′-methylcarboxyfluorescein) along with a nucleic acid molecule with a terminated primer. Subsequent incorporation would be blocked by the presence of the 3′-O-azidomethyl group protecting the 3′-hydroxyl group. After a washing step, TCEP is introduced to the sample to convert the 3′-O-azidomethyl group on the primer into a 3′-hydroxyl group, allowing incorporation of the next base by a replicating catalyst, e.g., polymerase enzyme, in the subsequent cycle. Reversible terminator nucleotides may be employed in conjunction with any of the fluorogenic nucleotides described herein.

The use of reversible terminator chemistry in combination with fluorogenic nucleotide substrates also allows the possibility of four-color synchronous sequencing-by-synthesis. By using the four reversible terminator nucleotide bases (dA, dT, dC, and dG) each labeled with a different fluorogenic dye, one could introduce all four bases to a nucleic acid sample simultaneously and determine the identity of the base incorporated into a nucleic acid sample in a given microreactor based on the color of the resulting fluorescent product. This would reduce the average number of cycles required to sequence a given template position, eliminate incomplete homopolymer synthesis, and decrease the rate of misincorporation due to the guaranteed presence of the correct base.

Additional reversible terminators are known in the art, e.g., as described in Bentley et al., Nature 2008 456:53-9.

### Enzymatic Signal Amplification

Enzymatic signal amplification can be employed to increase the number of fluorescent product molecules generated during base identification at a single template position. Sood et al. have coupled exonuclease to polymerase incorporation of fluorogenic nucleotide substrates (Sood et al. J. Am. Chem. Soc., 2005, 127, 2394-2395). When exonuclease is included in a primer extension assay employing fluorogenic nucleotides, every time a polymerase enzyme incorporates a base resulting in fluorescent product generation, an exonuclease enzyme removes that base from the extended primer allowing the polymerase enzyme to re-incorporate a base at the same position. This leads to the generation of even more fluorescent product. The process can be repeated many times by polymerase and exonuclease enzymes and can result in 1000-fold signal amplification. However, an exonuclease-resistant primer must be employed to prevent primer digestion past the template position of interest. This can be accomplished using a primer with a phosphorothioate bond. For example, one could combine exonuclease and polymerase to amplify the signal corresponding to the incorporation of a single base in which many cycles of primer extension and digestion are repeated in the presence of one fluorogenic nucleotide substrate such as dA4P-δ-3-O′-methylcarboxyfluorescein. If the next base on the template strand in a given microreactor is T, then a replicating catalyst, e.g., polymerase enzyme, will incorporate the complementary fluorogenic substrate resulting in the generation of the 3-O′-methylcarboxyfluorescein, a fluorescent product, in the presence of alkaline phosphatase.

However, an exonuclease enzyme would then remove the incorporated base, generating dAMP and allowing the replicating catalyst, e.g., polymerase enzyme, to re-incorporate dA4P-δ-3-O′-methylcarboxyfluorescein at the same position, generating more fluorescent product. After sufficient signal has been generated, the sample would be washed, and an unlabeled nucleotide substrate (corresponding to the same base that had just undergone multiple incorporation cycles) in which a phosphorothioate replaces the α-phosphate would be introduced (e.g. dATPaS):

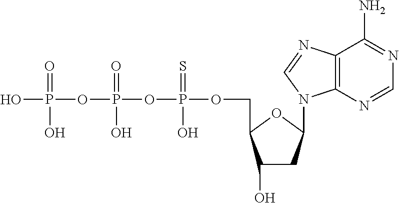

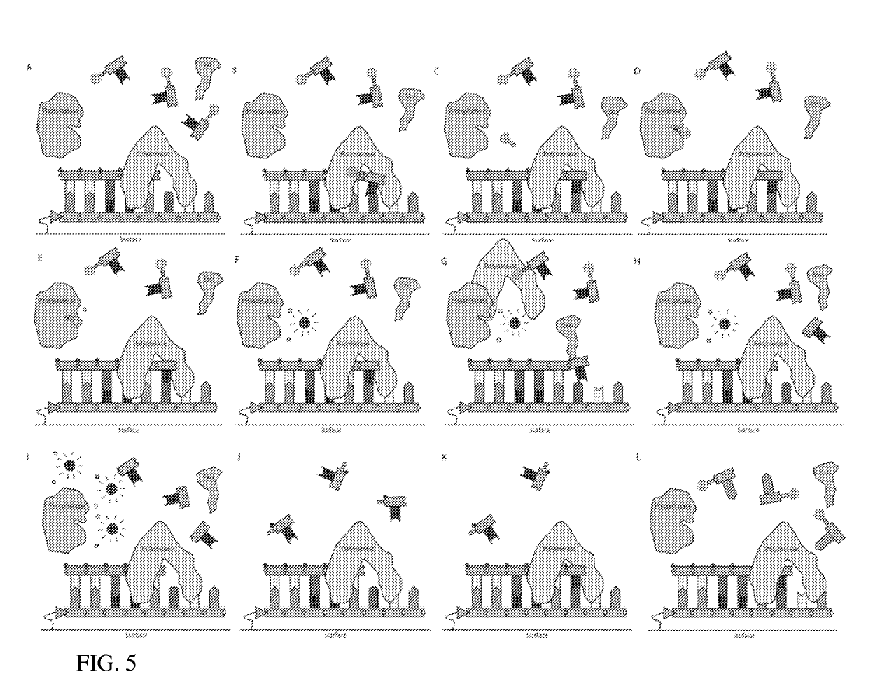

Many polymerase enzymes have been shown to incorporate phosphorothioate-modified nucleotides efficiently and with excellent fidelity. This step would be carried out in the absence of exonuclease or any fluorogenic nucleotide substrate, allowing primer extension to be completed. The newly extended primer would be indigestible by exonuclease because of the phosphorothioate bond formed by incorporation of dATPaS, allowing fluorogenic sequencing-by-synthesis with enzymatic amplification by exonuclease to continue for another cycle at the next template position with a different base (FIG. 5).

Homopolymeric regions also pose a challenge for synchronous fluorogenic sequencing-by-synthesis with enzymatic amplification which could be addressed by reversible terminator chemistry. Two modifications to the above scheme for enzymatic signal amplification would allow high accuracy homopolymer sequencing. The first is the use of a dideoxy fluorogenic nucleotide substrate instead of the typical deoxynucleotide fluorogenic substrate. For example, instead of using dA4P-δ-3-O′-methylcarboxyfluorescein as in the previous case, one would use ddA4P-δ-3-O′-methylcarboxyfluorescein as a fluorogenic substrate:

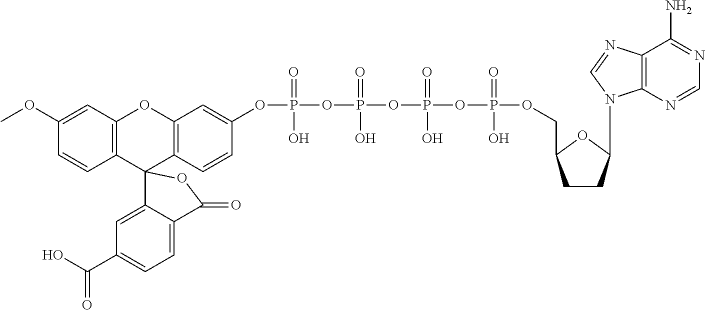 [SMILES: COc1ccc2c(c1)Oc1cc(OP(=O)(O)OP(=O)(O)OP(=O)(O)OP(=O)(O)OC[C@@H]3CC[C@H](n4cnc5c(N)ncnc54)O3)ccc1C21OC(=O)c2ccc(C(=O)O)cc21]

The absence of 3′-hydroxyl group on this fluorogenic substrate would prevent primer extension beyond the next template position. Secondly, in the subsequent step in which a phosphorothioate-modified nucleotide is incorporated to extend the primer and maintain exonuclease-resistance, a reversible terminator, phosphorothioate-modified nucleotide would be employed to simultaneously prevent primer extension beyond the next template position and primer degradation as in:

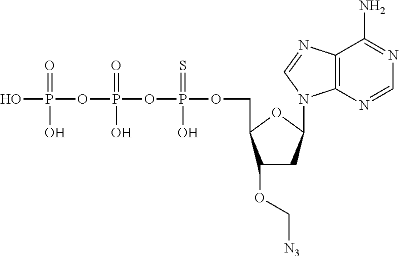

The protecting group on the 3′-hydroxyl can then be removed by TCEP to allow primer extension in a subsequent cycle. This procedure would allow enzymatic amplification and synchronous fluorogenic sequencing-by-synthesis without concern for incomplete primer extension against homopolymeric template regions, which can lead to errors. In addition, just as in the previously described implementation of reversible terminator chemistry, four-color synchronous fluorogenic sequencing with enzymatic amplification is also possible and would have similar advantages along with the possibility of sequencing with a small number of template molecules, even as few as a single template molecule.

### Detection

Incorporation of an individual nucleotide may be detected by detecting the light emitted from its corresponding label by any appropriate method. For fluorescent labels, one or more excitation sources may be employed, depending on the nature and number of labels. Methods for fluorescence detection are known in the art. Examples are conventional fluorescence microscopy, total internal reflection fluorescence microscopy, high inclined illumination microscopy, or parallel confocal microscopy (Lundquist et al. Optics Letters. 2008 33(9) 1026-1028). Additionally, simple lamp- or LED-based widefield illumination may be employed as a detection method. As described above, the methods of the invention may be employed in a multiplexed mode, where the sequences of multiple target nucleic acids are determined simultaneously, e.g., using a wide field of view detector such as a charge-coupled device (CCD) or multiple detectors.

The invention also includes use of a stage to move the microreactors relative to the detector. This allows for the sequential imaging of a portion of the microreactors. In this embodiment, a portion of the microreactors may be imaged, while other portions are receive reagents or wash solutions or are allowed to undergo template-dependent replication to release label prior to imaging. In some cases, the sample scanning stage can communicate with a detector in order to synchronize sample motion with data acquisition. For example, the motion of a stage can be used to trigger charge transfer from a time delay integration CCD detector (TDI-CCD).

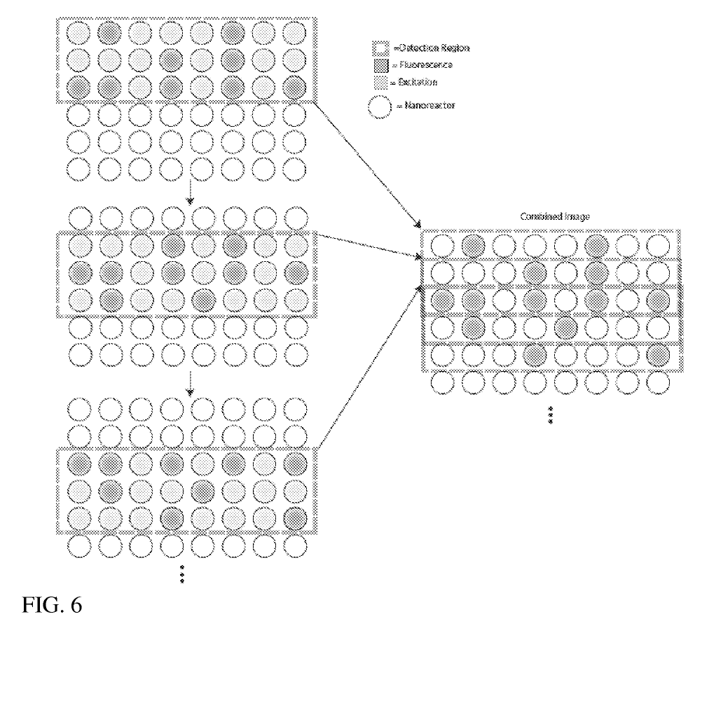

The illumination and detection geometries employed in fluorogenic sequencing ideally provide high sensitivity fluorescence detection and sufficient spatial resolution for identifying individual microreactors while maximizing the speed with which fluorescence signal can be recorded from each microreactor. In many cases, imaging a sample with a relatively small illumination area is critical, because scattering and autofluorescence background scale unfavorably with illumination area. Furthermore, microscope objectives and aspheric lens elements, which are particularly advantageous for achieving sufficient spatial resolution, limit the illumination area. By using fast sample scanning, one can acquire fluorescence data rapidly while still illuminating a relatively small field of view. In one embodiment, point scanning is employed to rapid fluorescence imaging of a microreactor array (FIG. 6). This method is analogous to point scanning in confocal microscopy where the natural imaging area at a given instant is a diffraction limited spot. In some cases, it may be advantageous for the imaging area to be smaller than that of a single microreactor, but collection of fluorescence signal need only occur when the illuminating beam passes through a microreactor. To achieve rapid point scanning of a microreactor array, one can combine fast sample scanning using a motorized or piezoelectric stage with fast beam scanning. Beam scanning can be accomplished by a number of means including galvo mirrors, resonant galvo mirrors, acoustooptic deflectors (AODs), electrooptic deflectors (EODs), spinning disks, lens translation, spatial light modulators, and other methods known in the art. In addition, multifocal microscopy can be applied using gratings or holographic optical elements to generate an array of foci or beams at the specimen plane of the microscope that correspond with the microreactor array. Depending upon the exact geometry, point scanning is compatible with either point or array detection. A single beam can be scanned with the fluorescence imaged onto a point detector such as a photodiode, photomultiplier tube (PMT), avalanche photodiode (APD), or single photon avalanche photodiode (SPAD). Alternatively, when multiple array elements are illuminated simultaneously with multiple beams or when the imaging can be temporally coordinated with point scanning, array detectors such as charge coupled device (CCD) cameras, electronmultiplication charge coupled device (EMCCD) cameras, complementary metal oxide semiconductor (CMOS) cameras, PMT arrays, photodiode arrays (PDAs), APD arrays, or SPAD arrays can be applied.

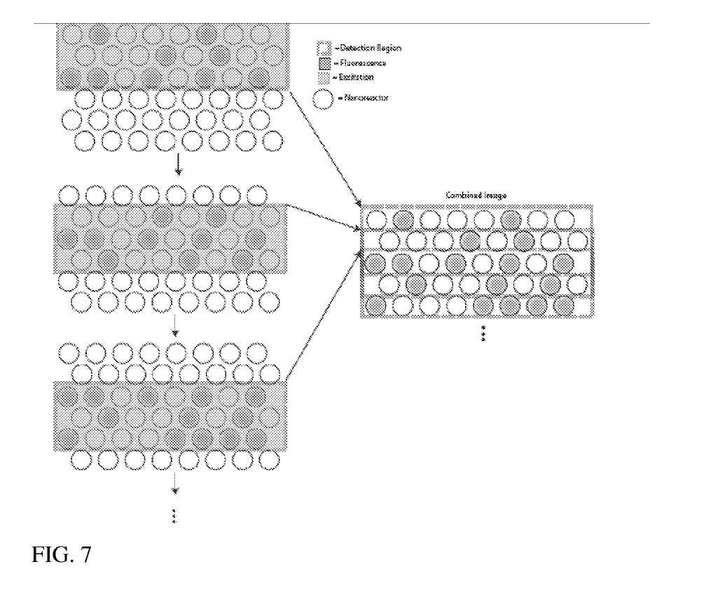

In another embodiment, line scanning high speed fluorescence imaging of a microreactor array. In a line scanning microscope, a rectangular beam illuminates one or more rows of microreactors in an array simultaneously (FIG. 7). Linear array detectors such as CCD cameras, EMCCD cameras, CMOS cameras, PMT arrays, PDAs, APD arrays, or SPAD arrays are suitable detector elements in this case. In one particular implementation, a beam with a rectangular profile illuminates a single row of microreactors in an array while the array is rapidly translated perpendicular to the long axis of the rectangular beam profile with a motorized translation stage. A variety of optical elements such as cylindrical lenses, engineered diffusers, spatial light modulators (SLMs), or slits can be used to generate this beam shape. An array detector with an aspect ratio similar to that of the beam profile can then be used to image the fluorophores trapped inside the illuminated microreactors.

There are several advantages to either line or point scanning fluorescence microscopy when compared to wide field, two dimensional image acquisition. Because the instantaneously illuminated area is small, the fluorescence and scattering background signals will be correspondingly small. Additionally, because a relatively small area is illuminated, the total excitation power required for a given power density is reduced relative to wide field imaging. For the same reason, the required number of elements or pixels in the array detector can be reduced which significantly increases the data acquisition rate and therefore the imaging throughput when the sample is rapidly scanned relative to the illumination. In contrast to wide are illumination and imaging, line or point scanning methods can allow for continuous, constant velocity sample motion, eliminating the need for rapid accelerations and decelerations of the sample between image acquisitions. Finally, these methods allow for constant illumination of the sample, because data are effectively acquired continuously rather than in discrete two dimensional images.

### Microfluidic Sample Preparation

In certain embodiments, target nucleic acids are purified from crude biomaterials (such as blood, tissue, etc.) using microfluidic techniques, which may be integrated with a system of the invention. Methods for isolating nucleic acids from cellular samples using microfluidic devices (i.e., devices having a channel with at least one dimension of less than 1 mm) are known in the art (e.g., U.S. Pat. No. 6,352,838). In addition, microfluidic devices may also be used to obtain either RNA or DNA from a single cell, e.g., as described Toriello et al., Proc. Natl. Acad. Sci., 2008 105(51), 20173-20178.

### Amplification

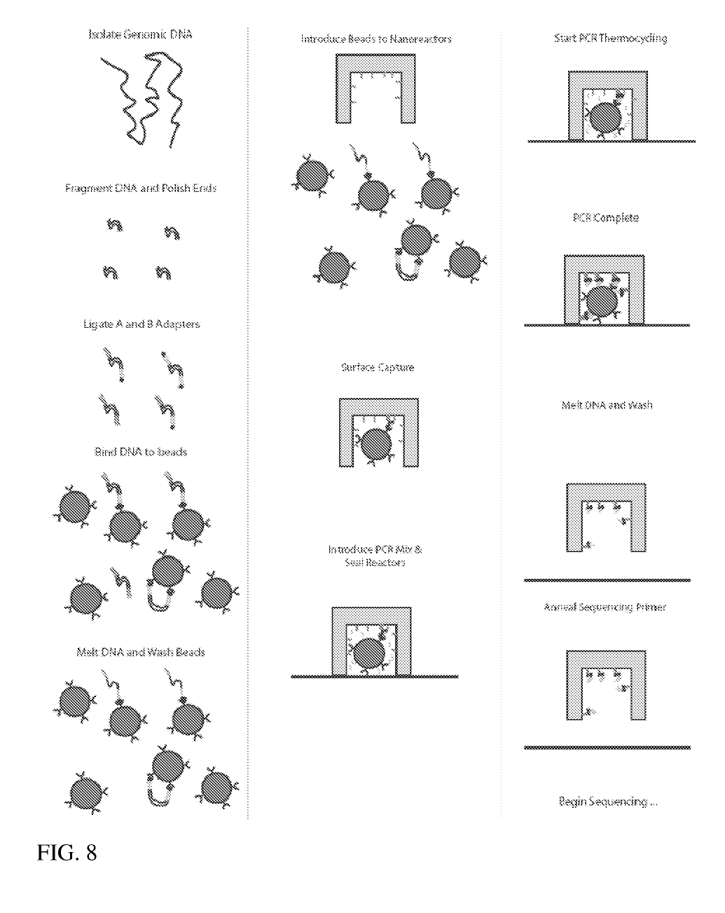

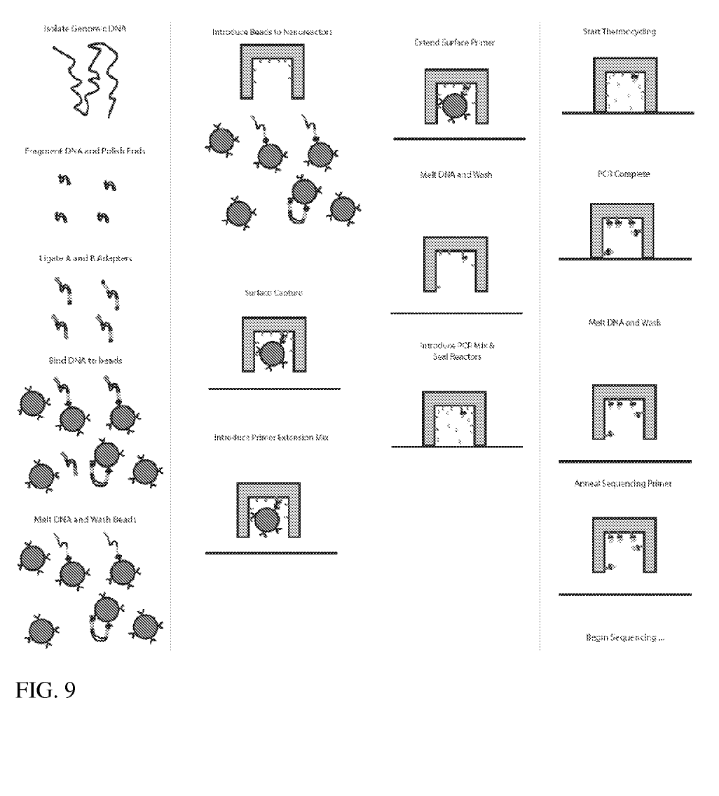

The invention also features methods of amplifying single copies of nucleic acids. In one method a single nucleic acid is bound, covalently or noncovalently, via one end to a bead. The bead is then introduced into a microreactor, as described herein, and the free end of the nucleic acid binds to the surface of the microreactor. The nucleic acid thus tethers the bead to the microreactor. The nucleic acid is then amplified using template-dependent replication to produce amplicons, as shown in FIGS. 8 and 9. Reagents necessary for amplification can be added by any appropriate manner, as described herein for sequencing. The reactions employed for amplification may be the same as those described herein for sequencing, although labels are not required during the amplification process. Exemplary amplification schemes include PCR, RCA, HRCA, and LCR. The amplicons produced may be bound to the surface of the microreactor or the bead. The bead may also be removed, e.g., to transfer the amplicons, e.g., for sequencing or other analysis, to another vessel. The bead may also be removed for analysis of nucleic acids bound to the microreactor. The bead also need not remain in the microreactor during amplification; it can be removed once the single copy of the nucleic acid is bound to the microreactor. In a related method, the single nucleic acid is introduced into the microreactor without being bound to a bead, e.g., by manual or automated pipette or dilute solution. The nucleic acid then binds (covalently or otherwise) to the surface of the microreactor and is amplified as described, where the amplicons are bound to the microreactor.

The amplification methods may be employed sequentially or in parallel with multiple nucleic acids, one per bead if beads are employed. For example, single nucleic acids may be bound to a plurality of beads, which are then deposited individually into microreactors. Beads that do not include a nucleic acid will not bind to the reactors and can be removed in a wash step. By repeating this process, a device having many microreactors, e.g., in an ordered array, can be partially (e.g., greater than 50%, 75%, 80%, 90%, or 95%) or completely filled with beads, i.e., super-Poisson loaded. Preferably, the beads and microreactors are sized so that only one bead can fit into a microreactor. Suitable beads for use with nucleic acids are known in the art. Typically, the beads will have a diameter between 0.1 and 50 nm. Single nucleic acids may also be added to partially (e.g., greater than 50%, 75%, 80%, 90%, or 95%) or completely fill the microreactors without being bound to a bead.

The nucleic acid may be single or double stranded, RNA, DNA, or a hybrid of both. The amplicons may be complementary in sequence, identical in sequence, or both. As will be understood, some variation in amplicon sequence may occur as a result of errors in template-dependent replication. The amplicons may also correspond to the full nucleic acid sequence or a portion thereof. Amplicons may also be produced with normaturally occurring modifications by appropriate selection of reagents, e.g., modified nucleotides or primers. For example, amplicons may be produced using primers that have moieties that can be covalently or noncovalently attached to a bead or microreactor. The method of attachment of the amplicon to a bead or microreactor may or may not be the same as that of the nucleic acid being copied.

Binding of nucleic acids to a bead or microreactor can occur by any known method, as described herein. Such methods include hybridization of an end of the nucleic acid to a complementary sequence of an oligonucleotide bound to the bead or microreactor. Other methods of attachment include using binding pairs, e.g., biotin/avidin and antibody/antigen. Nucleic acids may also be covalently attached to the beads or microreactor using known methods.

Single nucleic acids may be bound to a bead using any method known in the art. One method is described in FIG. 8. As shown, genomic double-stranded DNA may be isolated from a biological sample of interest. This DNA is then fragmented using one of a variety of methods (such as nebulization, ultrasonic shearing, or enzymatic cleavage) to generate fragments of approximately homogenous length, e.g., tens to hundreds of bases. The fragments are then enzymatically polished to generate blunt-ended fragments, which are ligated to two different types of DNA adapter fragments, A (with an A primer and complement A′) and B (with a B primer and complement B′). The 5′ end of the A primer contains a specific, chemically reactive moiety (e.g., protein or ligand, such as biotin) that allows for specific localization. This blunt ended ligation generates three different types of fragments: those with two A adapters, those with two B adapters, and those with one A and one B adapter. These fragments are then added, at very low concentration, to beads which allow immobilization of the A primer through its specific, chemically reactive moiety. The beads are in molar excess so that only one (or zero) piece of DNA binds to the bead. These beads are then washed, and the DNA is chemically or thermally melted off to produce single-stranded DNA (ssDNA) bound to the bead and to remove nonspecifically bound DNA. For example, this wash eliminates pieces of DNA with two B adapters (because they have no affinity for the beads). DNA with one A and one B adapter will leave one piece of ssDNA with a B′ primer sequence at the 3′ end. The beads are then introduced to a microreactor array. The B primer sequence is immobilized on the inner surface of the microreactor, e.g., covalently. Beads that have bound DNA fragments that contain two A adapters will not interact with the B primer on the microreactor surface, and therefore only pieces of DNA that include one A adapter and one B adapter will be immobilized in the microreactor. Additionally, the size of the bead may physically exclude more than one bead from entering the reactor, thus preventing the immobilization of more than one bead in the microreactor and ensuring that only one piece of DNA is present in the reactor. At this point, A primer, optional B primer (to increase the efficiency of the PCR), and PCR master mix is added to the reactors, and the reactors are sealed and then thermocycled to carry out PCR. Upon completion of the PCR reaction, the DNA is chemically or thermally melted to produce ssDNA. Then the reactors are opened and non-bound strands are removed, along with the bead. At this point, A primer is flowed into the chamber, and the ssDNA strands are primed for sequencing.

Another method is shown in FIG. 9. As shown, genomic double-stranded DNA may be isolated from a biological sample of interest. This DNA is then fragmented using one of a variety of methods (such as nebulization, ultrasonic shearing, or enzymatic cleavage) to generate fragments of approximately homogenous length, e.g., tens to hundreds of bases. The fragments are then enzymatically polished to generate blunt-ended fragments, which are ligated to two different types of DNA adapter fragments, A (with an A primer and complement A′) and B (with a B primer and complement B′). The 5′ end of the A primer contains a specific, chemically reactive moiety (e.g., protein or ligand, such as biotin) that allows for specific localization. This blunt ended ligation generates three different types of fragments: those with two A adapters, those with two B adapters, and those with one A and one B adapter. These fragments are then added, at very low concentration, to beads which allow immobilization of the A primer through its specific, chemically reactive moiety. The beads are in molar excess so that only one (or zero) piece of DNA binds to the bead. These beads are then washed, and the DNA is chemically or thermally melted off to produce single-stranded DNA (ssDNA) bound to the bead and to remove nonspecifically bound DNA. For example, this wash eliminates pieces of DNA with two B adapters (because they have no affinity for the beads). DNA with one A and one B adapter will leave one piece of ssDNA with a B′ primer sequence at the 3′ end. The beads are then introduced to a microreactor array. The B primer sequence is immobilized on the inner surface of the microreactor, e.g., covalently. Beads that have bound DNA fragments that containing two A adapters will not interact with the B primer on the microreactor surface, and therefore only pieces of DNA that include one A adapter and one B adapter will be immobilized in the microreactor. Additionally, the size of the bead may physically exclude more than one bead from entering the reactor, thus preventing the immobilization of more than one bead in the microreactor and ensuring that only one piece of DNA is present in the reactor. At this point, a reaction mixture including DNA polymerase and all four nucleotides is added to the reactors, and the surface-bound primer to which the single template DNA molecule is attached is extended. Upon completion of this initial primer extension reaction, the resulting double-stranded DNA (dsDNA) is melted, and the bead is washed away. Single-stranded RNA (ssRNA) could also be immobilized on the bead originally and captured on the microreactor surface in a similar fashion. In this case, a reaction mixture including reverse transcriptase and all four nucleotides would be added to the microreactors to reverse transcribe a complementary DNA template. The ssRNA-bead complex would then be melted (or the RNA digested) and washed away, just as in the DNA case. In both instances, the microreactor has many B primers on its inner surface along with a single copy of DNA that is complementary to the original template from the bead at the end of this process. A primer, optional B primer (to increase PCR efficiency), and a PCR mix are then added to the microreactors which are subsequently sealed and thermocycled to carry out PCR. Upon completion of the PCR, the DNA is chemically or thermally melted to produce ssDNA. The reactors are then opened, and unbound strands are removed. At this point, A primer is flowed into the chamber, and the ssDNA strands are primed for sequencing.

The methods exemplified in FIGS. 8 and 9 may also be employed with other types of nucleic acid, e.g., RNA or DNA from a different source. In addition, amplification techniques other than PCR may also be employed.

In alternative methods, the desired nucleic acid remains bound to the bead, which can be removed and transferred to another vessel for analysis or further manipulation. The adaptors employed may or may not include nucleotide sequences. If included, such sequences may or may not act as binding sequences for primers for amplification. Nucleic acids may also be prepared from by libraries or biological samples by other methods. For example, nucleases, e.g., restriction endonucleases, could be employed to cleave large nucleic acids into smaller fragments. The known sequence produced by such treatment could then be employed for direct attachment to a bead or microreactor or to an adaptor. Other methods of producing fragments of nucleic acids are known in the art. The methods may also be employed in the absence of a bead, where nucleic acids are modified as described for binding to a microreactor and subsequently amplified.

Washing and melting steps may be employed as necessary to produce the desired amplicons. For example, a melting step followed by washing can be employed to produce single stranded nucleic acids bound to the microreactor or bead. Alternatively, the amplicon may be double stranded. Washing steps may also be employed to remove nucleic acids that are not bound to the microreactor or bead

Rolling circle amplification may also be employed with or without additional amplification by PCR. For example, linear, rolling circle amplification (RCA) with a strand-displacing nucleic acid replicating catalyst, e.g., DNA polymerase, may be employed prior to microreactor surface capture to enhance the efficiency of surface capture and reduce the number of PCR cycles required to generate template copies for sequencing (Fire et al. Proc. Natl. Acad. Sci. 92, 4641-4645, 1995; Lizardi et al. Nat. Genet. 19, 225-232, 1998.). In cases where relatively small microreactors (e.g., with diameters less than 2 μm) are used, RCA may provide sufficient amplification without a subsequent PCR cycle. Pre-amplification with RCA has the added advantage of very high accuracy (Dean et al. Genome Res. 11, 1095-1099, 2001). In RCA, the accuracy of replication is independent of the accuracy of previous replications. Furthermore, any subsequent PCR cycles would occur on multiple copies of target DNA template instead of a single molecule, further reducing the propagation of error. Additionally, RCA can be conducted with a highly processive, strand-displacing nucleic acid replicating catalyst, such as φ29 DNA polymerase, which has strong error-correcting exonuclease activity (Dean et al. Genome Res. 11, 1095-1099, 2001).

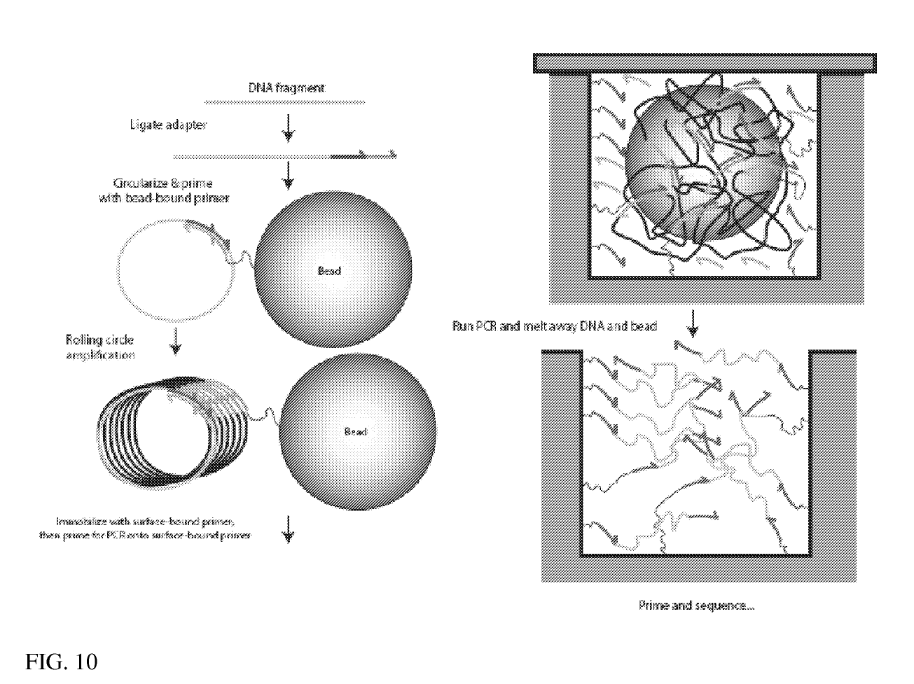

In one embodiment, a ssDNA template is 5′-phosphorylated with a polynucleotide kinase and circularized with CircLigase (Epicentre). Alternatively, an adapter-ligated 5′-phosphorylated ssDNA template is annealed to a primer that joins the two template ends, allowing circularization by a double-stranded DNA ligase. The circular DNA is captured on a bead by a covalently or biotin-streptavidin bound primer and replicated linearly by φ29 DNA polymerase by RCA (FIG. 10). For a 100-base DNA template, φ29 DNA polymerase generates about one copy every two seconds, and a 10 kb amplicon containing 100 copies of the template can be generated in ˜3-4 minutes without thermocycling (Nallur et al. Nucl. Acids. Res. 29, 118, 2001; Sato K. et al. Lab on a Chip. 10, 1262-1266, 2010). Because the resultant amplicon is immobilized on a bead, multiple templates can be amplified simultaneously in a single vessel either in solution or on a surface. If the amplicon-bound beads are only slightly smaller than the microreactors, super-Poisson loading of a microreactor array can be achieved. If complementary capture primers are immobilized on the inner walls of the microreactors, amplicon-bound beads can be captured selectively, avoiding the immobilization of beads that lack an amplified DNA template. For 5-μm diameter microreactors, it is preferable to have 3,000-10,000 copies of DNA template per microreactor. Hence, 5-10 cycles of microreactor PCR can be employed to generate sufficient primed, ssDNA template, e.g., bound to microreactor walls, for sequencing. For smaller microreactors (e.g., <2 μm in diameter), 500-1,000 copies of DNA template per microreactor may be employed for sequencing. In this case, 3-5 cycles of microreactor PCR may be employed. Alternatively, sufficient template for sequencing in small microreactors can be generated solely by the RCA reaction. For example, a 100-base DNA template, one can generate ˜700 copies via RCA before φ29 DNA polymerase dissociates given its processivity of ˜70,000 bases (Dean et al. Genome Res. 11, 1095-1099, 2001). Larger RCA products have also been generated using a molar excess of X29 DNA polymerase (Nallur et al. Nucl. Acids. Res. 29, 118, 2001).

In a second embodiment, either pre-amplification with RCA followed by microreactor surface capture or single template microreactor surface capture is used to immobilize template-bead complexes in microreactors. In a subsequent step, two primers which hybridize in tandem to the DNA template are used to initiate and propagate hyperbranched rolling circle amplification (HRCA) in sealed microreactors. HRCA is similar to RCA in that it is isothermal and requires strand-displacement, but it results in exponential amplification rather than linear amplification and generates multiple dsDNA products with various lengths (FIG. 11), some of which become dissociated from the replication center. HRCA has been shown to generate amplicons with greater efficiency than PCR in some cases, and could be conducted on DNA templates immobilized in microreactors to generate amplicons for sequencing without the need for thermocycling. Sequencing primers could be immobilized on the microreactor surface, allowing surface capture of the HRCA products.

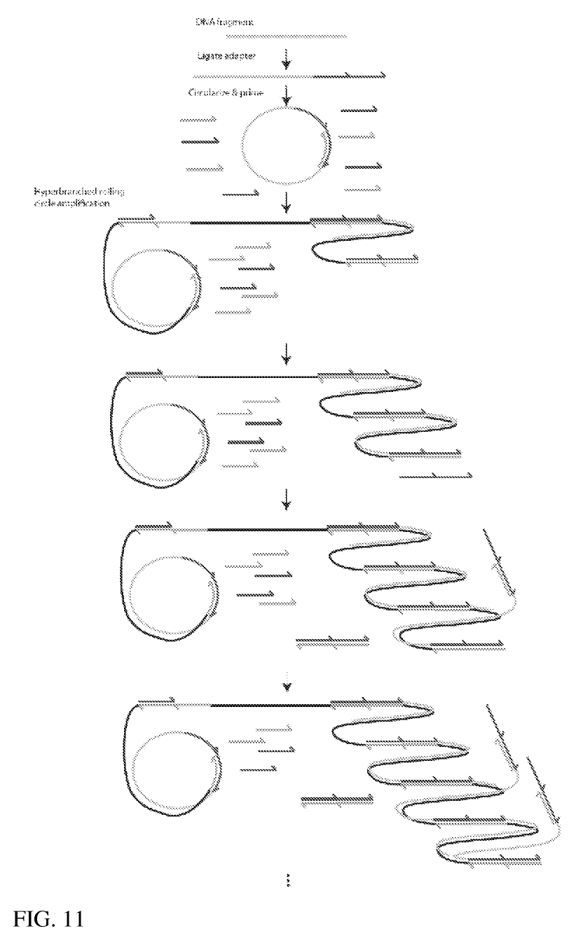

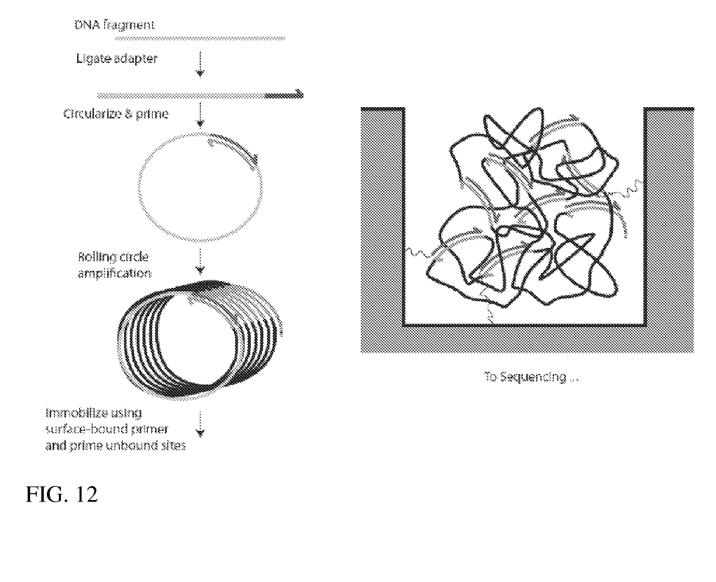

Alternative applications of isothermal amplification involve generating large amplicons before DNA immobilization in a microreactor array. In one example, linear RCA is carried out to produce thousands of contiguous template copies from multiple circular DNA sequences in a single vessel. Because RCA can generate micron-sized ssDNA products (Sato K. et al. Lab on a Chip. 10, 1262-1266, 2010), these templates can be super-Poisson loaded into micron-sized microreactors without attachment to beads (FIG. 12). Although this method eliminates beads from the sample preparation, it has the disadvantage that such large DNA constructs may be mechanically unstable.

### Sample Loading

Microreactors can be substantially loaded with a single type of nucleic acid, either as a single copy, as multiple, individual copies, or as multiple concatemeric copies. As described herein, microreactors can be loaded with single copies of nucleic acids by employing a dilute solution of the sample so that on average each microreactor contains zero, one, or only a few copies. Such methods allow sample loading based on a Poisson distribution. Methods for super-Poisson loading may also be employed to load microreactors. For example, physical exclusion by employing microreactors size to fit a single nucleic acid containing bead or a single concatemeric nucleic acid. Individual delivery of sample to microreactors, e.g., using a pipetting robot, may also be employed. In certain embodiments, loading by automated or manual pipette is specifically excluded.

An alternative class of super-Poisson loading methods involves the saturation of a controlled number of binding sites for a single nucleic acid molecule or population of amplicons without the use of physical exclusion. These techniques avoid the use of polymer or superparamagnetic beads with complex surface chemistries and reduce the amount of time required to prepare a sample for sequencing. In general, the methods rely on the binding of a controlled number of moieties to the surface of a microreactor, and the provision of a suitable number of nucleic acids to bind to substantially all of the surface moieties, e.g., by hybridization, by other non-covalent interaction (e.g., biotin-streptavidin or antibody-antigen), or by covalent reaction.

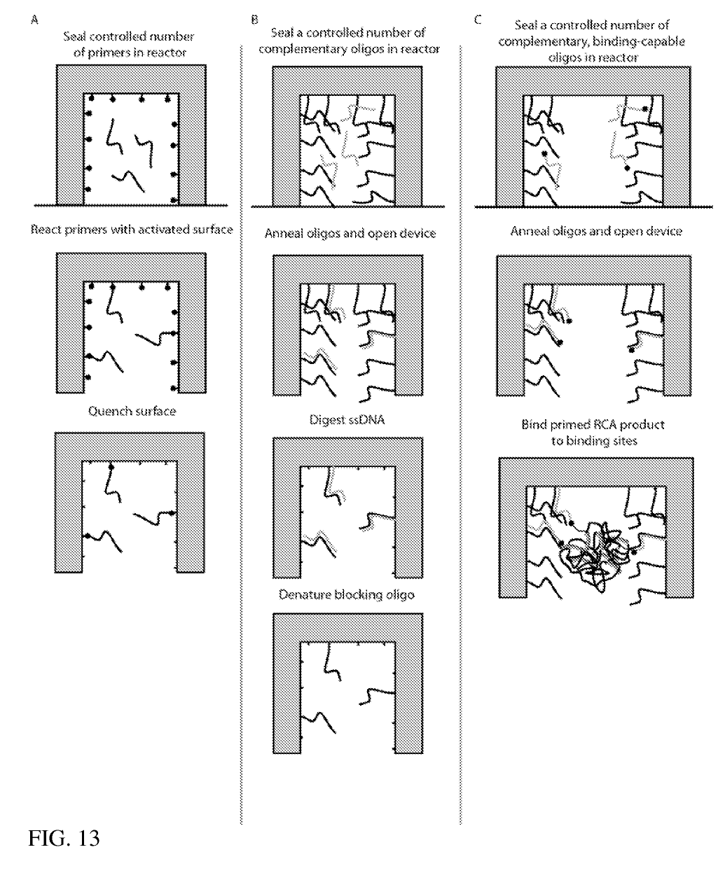

In one embodiment, microreactors are functionalized by patterned deposition of a reactive silane on the inner walls, e.g., using one of the methods described below. Silanization allows covalent attachment of 5′-modified DNA primers to the microreactor surface. Microreactor surfaces can be functionalized with a variety of reactive groups such as thiols, amines, aldehydes, maleimides, or succinidimidyl esters for reaction with DNA primers that are 5′-modified with appropriate reactive groups. In particular, one can construct a PDMS flow cell containing a silanized PDMS microreactor array and introduce 5′-modified DNA primers to the microreactors at a known concentration. By rapidly sealing the microreactor array, the number of 5′-modified DNA primers trapped in each microreactor can be controlled such that a fixed number of primers react with the silanized surface. In this manner, one can control the number of DNA primers that are covalently attached to the inner walls of the PDMS microreactors. For example, if one has microreactors with a volume of 80 fL, trapping a 200-nM solution of 5′-modified DNA primers in the microreactors deposits about 10,000 molecules on the inner walls of the microreactor, if the surface coupling reaction goes to completion. These surface immobilized primers can serve as either forward or reverse primers for subsequent amplification steps. This case is shown schematically in FIG. 13A.

One can achieve super-Poisson loading of amplified sequencing templates in a microreactor array by first trapping single template molecules in PCR primer-coated microreactors at a concentration such that almost all microreactors contain either zero or one template DNA molecule. Alternatively, one could load a concatemeric amplicon resulting from rolling circle amplification (RCA) at a similar concentration. On-chip PCR can then be used to amplify the trapped template molecules. If the templates are circularized, then hyperbranched rolling circle amplification (HRCA) can be used to amplify the trapped template molecules isothermally and nonlinearly. Because one of the two PCR or HRCA primers is immobilized on the microreactor surface, the template (or its complement) will be covalently attached to the microreactor surface at the conclusion of on-chip amplification. If a sufficiently large number of PCR cycles are run or if an isothermal HRCA reaction is run for a long enough time, substantially all of the immobilized primers in template-containing microreactors will be covalently linked to a template (or complement) copy. This process can then be repeated when single template molecules or concatemeric amplicons are again trapped in the microreactor array at a concentration such that almost all microreactors contain either zero or one DNA molecule in solution. Some of the microreactors that already have surface immobilized template molecules will trap a new template molecule in this process. However, because there are no primer sites remaining on the surface because of previous amplification cycles, no amplification of the newly introduced template molecule will occur that results in surface-immobilized copies of the new template. Microreactors that contain a newly introduced template molecule but that did not contain a template molecule in the previous amplification cycles will contain surface-immobilized copies of a template molecule following a second set of amplification cycles. This process can be repeated several times until a desired fraction of microreactors contain clonally amplified, surface-immobilized DNA templates for sequencing.

In a second embodiment, one can employ multiple rounds of PCR or HRCA to saturate surface-immobilized primers, where the surface density and therefore copy number of surface-immobilized primers is controlled by hybridization rather than covalent surface chemistry. In one case, the inner walls of microreactors are functionalized with a reactive group by silanization followed by covalent immobilization of 5′-modified oligonucleotides (Oligo A). A complementary oligonucleotide (Oligo A′) can then be trapped in the microreactors at a concentration that limits the number of copies that hybridize to Oligo A. This copy number is preferably approximately the number of DNA templates required for sequencing. At this point, a fraction of the surface-immobilized DNA will be double-stranded. The remaining single stranded oligonucleotides on the surface can be eliminated selectively using, for example, Exonculease I. By digesting the remaining, unextended Oligo A and subsequently melting away the Oligo A′, one can generate a microreactor surface with the desired Oligo A copy number. The remaining copies of Oligo A can then be used as forward or reverse primers for PCR or HRCA while a second oligonucleotide (Oligo B) serves as the opposite primer. The microreactor array can be Poisson-loaded with single template molecules or concatemeric pre-amplicons multiple times, and PCR or HRCA can be used to saturate the surface-immobilized Oligo A following each loading cycle. This method is shown schematically in FIG. 13B.

Alternatively, Oligo A is a particularly short oligonucleotide (i.e., too short to be hybridized to complementary DNA at the high temperatures involved in PCR). These short oligonucleotides can be used to capture Oligo A′ at room temperature or below. Oligo A′ can be trapped in the microreactors to control the surface density of hybridized Oligo A′, as described above. The short Oligo A can then be extended using DNA polymerase and an appropriate reaction mixture, generating a full-length complement of Oligo A′ on the surface. If necessary, a single-stranded exonuclease such as Exonuclease I could then be used to digest the unextended Oligo A remaining on the surface. Because Oligo A is short, exonuclease digestion can be expected to proceed with higher efficiency than in the above case where Oligo A must be sufficiently long to serve as a primer in PCR. In some cases, it may not be necessary to digest the remaining Oligo A because Oligo A is too short to participate in PCR. Multiple rounds of Poisson-loading single template molecules or concatemeric pre-amplicons can be employed in combination with multiple rounds of PCR to achieve super-Poisson immobilization of amplified templates in the microreactor array.

In a third embodiment, saturation-based loading of a microreactor array is accomplished without the use of multiple rounds of PCR or HRCA. In this scheme, the inner walls of microreactors are functionalized with a reactive silane, and 5′-modified DNA (Oligo C) is covalently attached to the microreactor surface. A solution containing a second set of 5′-modified primers (Oligo C′) that are complementary to the surface-immobilized primers are then trapped in the microreactor array at a concentration such that each microreactor contains a relatively small number of Oligo C′ (e.g., 10 or 100 or 1,000 copies). A particularly useful 5′ modification for Oligo C′ in this case is a dual biotin. Dual biotinylated oligonucleotides that are bound to streptavidin can be thermally melted from their complements without dissociating from streptavidin. After trapping Oligo C′ at a certain concentration, Oligo C′ will anneal to Oligo C, and each microreactor will have a very similar number of, for example, dual biotin moieties immobilized to their surfaces. In the case that dual biotin moieties are chosen as the modification for Oligo C′, the resulting microreactor surfaces can then be saturated with streptavidin. Because the dual biotin modification binds two of streptavidin's four biotin binding sites, the inner surface of each microreactor will be functionalized with a narrowly distributed number of streptavidins each with two binding sites available. Alternatively, streptavidin can be covalently attached to the microreactor surface through its reactive thiols or amines in sealed microreactors to control the number of surface-immobilized streptavidins. Streptavidin could also be attached through a covalently immobilized biotin whereby either the number of immobilized biotins or streptavidins is controlled by trapping a solution of fuctionalized biotin or streptavidin at a certain concentration in the microreactor array. This method is shown schematically in FIG. 13C.

At this point, a set of circularized DNA templates for sequencing can be primed and amplified using isothermal RCA. The RCA reaction will copy not only the DNA template for sequencing, but also at least two primer sites for further amplification and sequencing. A dual biotinylated primer Oligo D can be annealed to multiple sites on the concatemeric RCA product. Preferably, a single RCA product will accommodate the hybridization of more functionalized copies of Oligo D than there are streptavidin binding sites in each microreactor.

The RCA products, which are now multiply functionalized by hybridization to Oligo D with, for example, dual biotin, can be introduced to the microreactor array and trapped in individual microreactors by sealing such that the vast majority of microreactors have either zero or one RCA product. Following a short incubation, the Oligo D-hybridized RCA products containing several dual biotin moieties will saturate the limited number of streptavidin binding sites on the microreactor surface. When additional RCA product is introduced to the microreactors and trapped, microreactors that already contain a surface-immobilized RCA product molecule will be unable to accommodate the surface capture of an additional RCA product molecule because all of its binding sites are saturated. However, microreactors that do not already contain a surface immobilized RCA product molecule will be able to capture one, and all of its surface binding sites will be saturated during a brief incubation. This process can be repeated until a sufficient number of microreactors contain single RCA products. In the case that the number of template copies produced by RCA is sufficient for sequencing, the RCA products can either be copied onto the microreactor walls by DNA polymerase or sequenced directly. Preferably, this second DNA polymerase would have minimal strand-displacement activity and negligible 5′-to-3′ exonuclease activity to maximize the uniformity of template replication. If further amplification is required, either on-chip PCR or HRCA can be used to amplify the RCA product onto the microreactor walls using the remaining surface-immobilized primers (Oligo C).

Although discussed with respect to particular surface reagents, microreactor materials, nucleic acids, and amplification techniques, the super-Poisson loading methods of the invention can be adapted for use of other microreactor materials, reagents for binding moieties to the surfaces, nucleic acids, and amplification techniques, as described herein. Such methods may be repeated as needed to partially (e.g., greater than 50%, 75%, 80%, 90%, or 95%) or completely fill the microreactors without being bound to a bead.

### Thermocycler

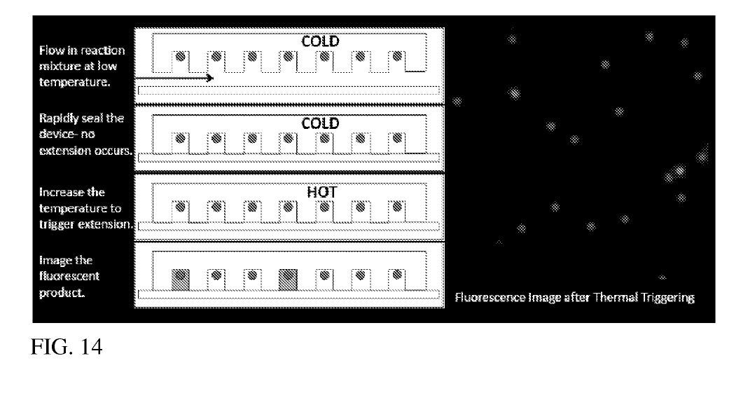

Control over the temperature of microreactor arrays is often necessary for both on-chip amplification and nucleic acid sequencing. Just as in conventional PCR, microreactor PCR requires rapid thermocycling to melt and re-anneal target DNA molecules repeatedly. Thermocycling is also beneficial to fluorogenic DNA sequencing in microrcactors. In general, when a sequencing reaction mixture is introduced to an unsealed microreactor array, the resulting primer extension reactions may start immediately, before the microreactor array is sealed. Depending upon the kinetics of nucleotide incorporation, a certain amount of fluorescent product may not be localized to the appropriate microreactor. This decreases the signal-to-background ratio and leads to cross-talk between microreactors. To minimize fluorescent product loss, low concentrations of fluorogenic nucleotides can be employed. At low concentrations, the microreactors contain relatively few nucleotide molecules, limiting the number of incorporation events that can occur each time the array is loaded. Because of the low concentration, multiple introductions of nucleotide to the microreactors may be needed to complete one cycle of sequencing. Higher density microreactor arrays containing smaller microreactors are more susceptible to this issue. As an alternative to low concentrations, the sequencing reaction mixture may be introduced at low temperatures, e.g., 15° C. to −20° C., where the nucleic acid replicating catalyst, e.g., DNA polymerase, has low activity. Once the microreactor array is sealed, the system can be raised to a temperature where the nucleic acid replicating catalyst, e.g., DNA polymerase, is highly active, e.g., 20° C. or above (for example, up to 95° C.) (FIG. 14). Temperatures employed will generally be those between the freezing and boiling point of the sequencing mixture. Besides providing a means of controlling sequencing, temperature control of sequencing has a number of additional advantages. For example, at room temperature, most DNA polymerases have difficulty extending a primer through regions of secondary structure. By cycling to temperatures greater than 50-60° C., most secondary structure in a DNA template is melted. Thermophilic DNA polymerases are particularly useful as they typically exhibit negligible activity below 4° C. and are highly active above 40° C.

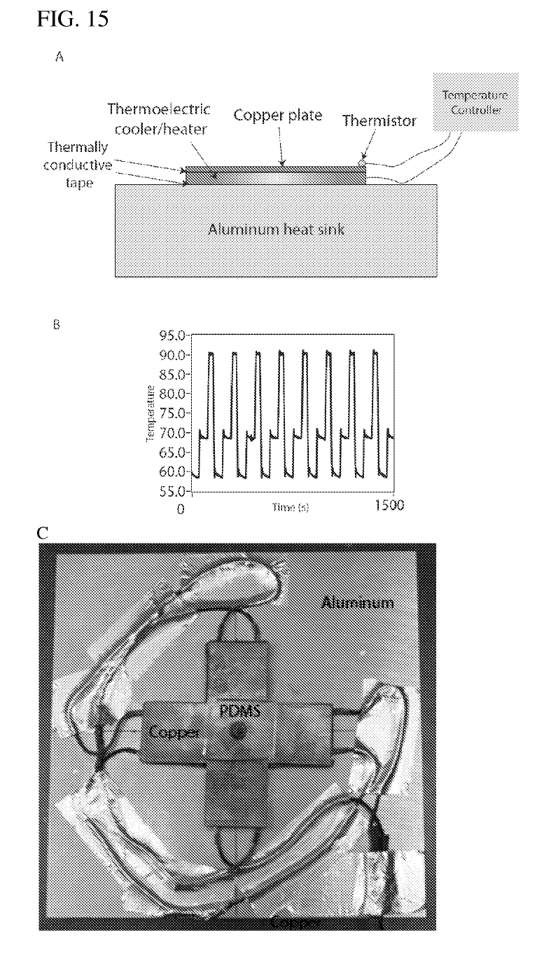

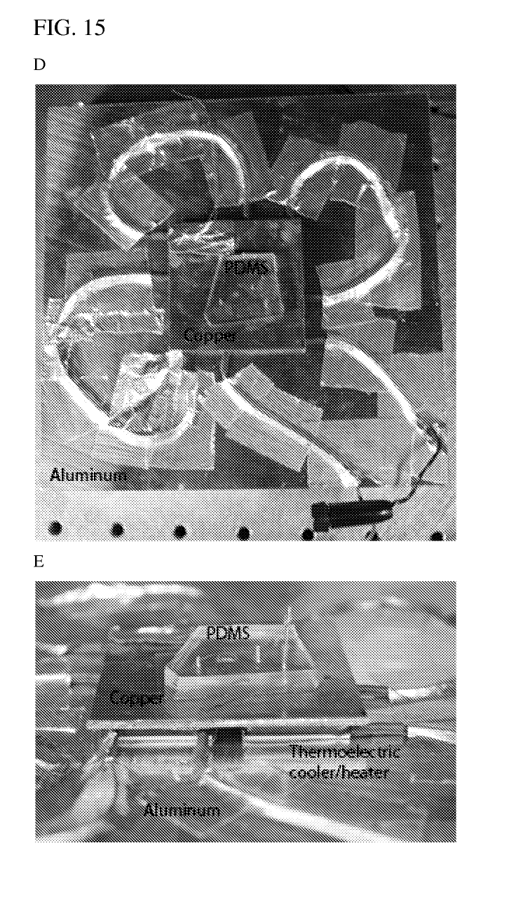

As shown in FIGS. 15A-E, a thermoelectric heating and cooling device was assembled from four Peltier devices (TE Technology) connected in series to an electronic temperature controller (TE Technology) with PID feedback and a LabVIEW interface that references a thermistor. The four Peltier devices are coupled to a large aluminum heat sink (bottom) and a copper plate (top) with thermally conductive tape. A microreactor array device with microfluidics can be mounted on the copper plate for thermocycling as shown in FIGS. 15A and 15C-E. This device can be readily mounted on an epifluorescence microscope. The Peltier devices are arranged so that a microscope objective can be inserted through the center of the device, and a hole in the copper plate allows imaging of the microreactor array. FIG. 15B shows typical thermal cycles achievable with this device.

### Dephasing—Incomplete Extension and Carry Forward

In order to obtain accurate sequencing data with long readlengths, the synchrony of nucleotide addition in a clonal population of nucleic acids is maintained. If some subset of nucleic acids to be sequenced does not incorporate the correct fluorogenic substrate when it is probed, this subset will be dephased from the rest of the population. This “incomplete extension” type of dephasing can occur either because the amount of time allowed for incorporation was insufficient, or because of a lack of substrate molecules within the microreactor to allow all possible incorporation events to occur. In either case, some population will be “behind” in the sequencing relative to the rest of the population, causing spurious signal and decreasing the overall signal from the synchronized population. Homopolymeric sequences are especially likely to suffer from this incomplete extension.

Alternatively, if all of one fluorogenic substrate species is not fully washed from the reactors before the next nucleotide species is introduced, then a population of nucleic acids being sequenced may, depending on the next base of the sequence, incorporate some of the contaminating substrate species. This “carry forward” type of error will cause some population to be “ahead” of the rest of the population, will likewise cause spurious signal in subsequent probe cycles, and will also decrease the signal from the synchronized population. To address this type of dephasing, the microreactors are efficiently washed between probe cycles to eliminate any contaminating nucleotide. However, stringent washing of a flow chamber is challenging, because liquid at the surface of the chamber does not flow rapidly because of the no-slip hydrodynamic boundary condition at the surface of the flow device. One way to increase the stringency of washing is to add an enzyme that efficiently digests the substrate molecule without generating spurious signal. For example in pyrosequencing, apyrase can be introduced to eliminate nucleotides. Similar enzymatic washing could be employed in the present invention.

The sealing of the device, either with conformal, physical sealing against an elastic material, or with an immiscible fluid, allows for a simple and effective solution to this washing problem. If a flowcell housing microreactors is fully sealed, or the sealing fluid is entirely replaced with a second immiscible fluid, then contaminating nucleotides in solution have necessarily been removed from the flowcell by physical exclusion. When new aqueous reagents are flowed into the flowcell they fully replace the previous liquid in the flowcell, eliminating hydrodynamic difficulties in washing. The only volumes, then, which must be washed are the microreactors themselves, which are generally small enough such that diffusion exchanges the contents of the microreactor on the order of milliseconds. Also, multiple conformal sealing rounds may be used to eliminate small residual contaminants that diffuse out from the microreactors.

Signal analysis methods that attempt to compensate for spurious signals generated by carry forward and incomplete extension dephasing are also well known in the art and could be used to increase the effective readlength and improve the accuracy of this technique.

### Combinations of Methods

The amplification, sample loading, and other techniques described herein may be employed with any suitable method for sequencing or otherwise assaying nucleic acids. The amplicons can be sequenced using the methods described herein; however, the amplification method may also be employed with any technique that benefits from the production of multiple copies of a nucleic acid. In certain embodiments, the methods may be used as an alternative to emulsion PCR. Other sequencing techniques that may be employed in connection with the amplification and sample loading aspects of the invention include other sequencing methods that employ fluorescent detection (e.g., as described in WO 01/94609), chemiluminescent detection, and electrical detection. For example, the microreactor amplification method could also be used in pyrosequencing in a picotiter plate (U.S. Pat. No. 7,244,559) or sequencing by ligation (U.S. Pat. No. 4,942,124 and U.S. 2008/0003571). The amplicons could also be employed in sequencing methods that rely on solid state or bridge PCR (U.S. 2009/0093378) or methods relying on spatial arrangement of nucleic acid or nucleic acid-coated beads over semiconductor-based sensors or field effect transistors (FETs) (U.S. 2009/012758 and U.S. 2009/0026082). For example, electrical detection in sequencing may employ field effect transistors that act as chemical sensors, such as chemFETs and ion-sensitive FETs (ISFETs). Such detection schemes employed with sequencing are described in U.S. 2009/0026082, which is hereby incorporated by reference. In a specific example, ISFETs detect changes in pH after incorporation of a nucleotide into a replicating nucleic acid. Microreactor-based amplification could also be linear, making it directly applicable to sequencing-by-hybridization technologies, as described in U.S. 2009/0264299.

The embodiments described in the following examples may be employed generally in the invention as described herein.

### Example 1

One method to generate arrays of micron and sub-micron scale reactors for confinement is the use of sub-micron lipid vesicles to entrap DNA, substrate, DNA polymerase, and phosphatase. We then immobilize these microreactors on the coverslide of a fluorescence microscope (Okumus et al. Biophys. J. 2004, 87(4), 2798-2806).

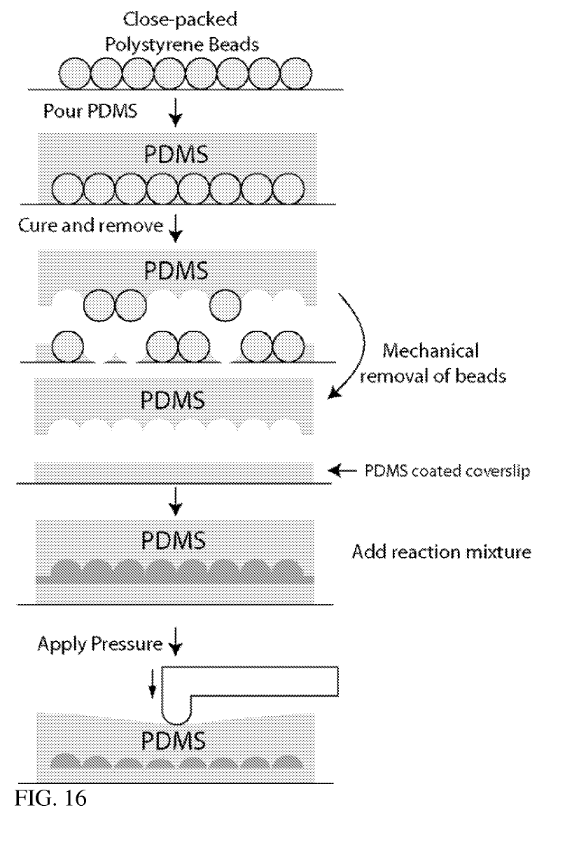

More uniform and controllable microreactors may also be generated through a variant of so-called nanosphere lithography (Hulteen et al. J. Vac. Sci. Technol. A 1995 13(3), 1553-1558) (see FIG. 16). In brief, we evaporate 500 nm to 2000 nm polystyrene or glass beads on glass slides to create a close-packed monolayer of beads. Then we pour PDMS onto these close-packed regions and cure the PDMS in a 60° C. oven overnight. The cured PDMS can then be peeled away from the glass, and impregnated beads removed mechanically. This process produces a portion of PDMS with a pattern of nanoscale indentations reminiscent of a honeycomb. Then, this PDMS pattern of dimples was pressed against a PDMS spin-coated coverslip to generate a regular array of microreactors that contain on the order of 5 to 0.1 fL. We are able to trap dye in these microreactors and image the dye with a two-color TIRF microscope, as shown in WO 2010/017487.

An alternate approach utilizes standard nanofabrication techniques to generate femtoliter-sized indentations in PDMS, poly(methyl methacrylate) (PMMA), or quartz, which we can seal against the surface of a coverslide (Rondelez et al. Nature Biotechnology. 2005, 23, 361-5 and Jung et al. Langmuir. 2008, 24, 4439-4442).

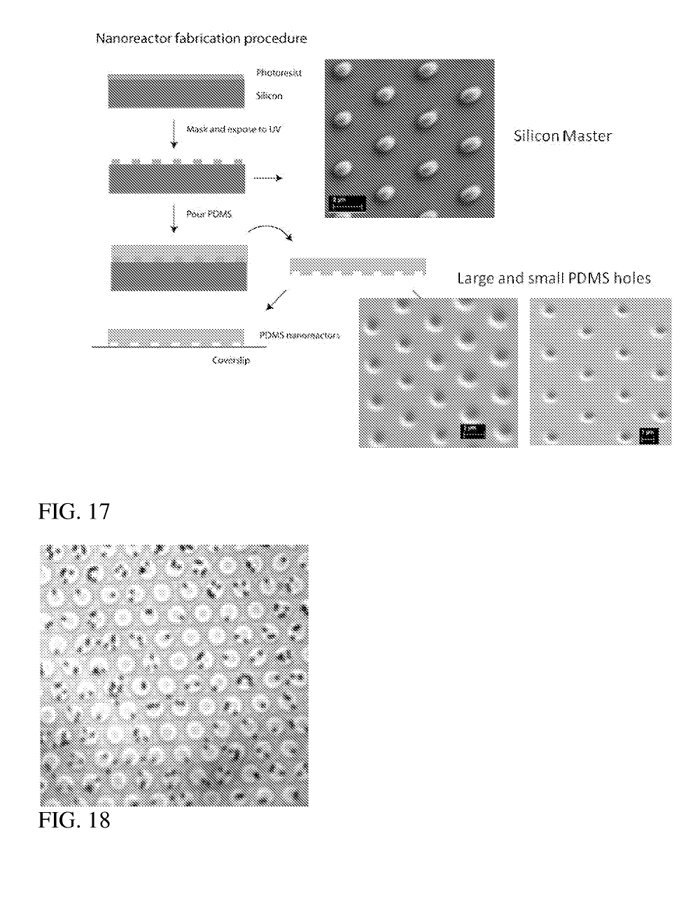

To improve the sealing characteristics of PDMS microreactors, we used these standard photolithographic methods to construct a microreactor array with wall thickness of greater than 1 micron. First, a flat 3 inch silicon wafer was coated with 0.5-1.5 microns of SU-8 2 photoresist and prebaked for 60 seconds at 65° C. and then 60 seconds at 95° C. Next, this photoresist was exposed through a patterned, chrome-on-glass photomask to UV light, which cross links the photoresist. This wafer is then post baked (identically to the prebake step) and developed, resulting in a resist-on-silicon master (FIG. 17). Finally, PDMS was poured onto this master, cured, and then used in experiments (FIG. 17). We have created ˜0.5, ˜1, ˜1.5, ˜2, ˜5, and ˜20 micron diameter reaction chambers using these methods.

To reduce nonspecific absorption of proteins and other species, PDMS was coated with an amorphous fluoropolymer CYTOP (perfluoro(1-butenyl vinyl ether) homocyclopolymer from Asahi Glass Co.,) by spincoating and baking at 75° C. for 15 minutes and 145° C. for 15 min. Then the CYTOP was coated with Pluronic F-108 (in the reaction solution), which spontaneously forms a polyethylene glycol brush on the surface of the microreactor because of hydrophobic interactions of the poly(propylene glycol) portion of the copolymer. We observed that this surface treatment prevents the adsorption of single fluorescently labeled protein molecules, thus eliminating the need for high concentrations of blocking protein (such as BSA), as shown in WO 2010/017487. The treatment also renders PDMS hydrophilic. Alternatively, moderate concentrations of BSA (1 mg/mL) can be used to block the PDMS.

Dyes such as DDAO and resorufin may diffuse through PDMS microrcactors, escaping the reactors in a timescale of seconds to minutes. Dyes with local negative charge may be efficiently trapped in PDMS microreactors for long timescales, e.g., on the order of hours (see, e.g., Rondelez, Y. et al. Nat Biotech 23, 361-365 (2005)). We demonstrated that the addition of a sulfonate group to DDAO, e.g., 6-sulfo-DDAO, provides the dye molecule with a local negative charge and eliminated diffusion of this dye through PDMS. This finding confirms that dyes with local negative charge were trapped in the PDMS microreactors.

We also treated PDMS microreactors with a stable fluorocarbon fluid (such as Fluorinert FC-43 and FC-770, 3M). By treating the PDMS with these compounds, we reduced the incidence of evaporation of the liquid phase within the reaction chambers and also reduced diffusion of uncharged substrates within the PDMS.

Alternately, microreactors are constructed out of different materials, such as fluorothermoplastics like THV 220 (3M), or PDMS can be coated with other impermeable materials to block the diffusion of non-charged dye species. Material coatings such as CYTOP also reduced or eliminated the diffusion of even non-charged dye molecules. Additionally, coating a CYTOP layer with a fluorocarbon liquid (such as Fluorinert FC-43, 3M) allows more robust sealing of microreactors by filling in small imperfections in the CYTOP layer.

In addition, vapor phase treatment of the oxidized coverglass surface with a variety of reactive silanes such as 1H, 1H, 2H, 2H-perfluorooctyltrichlorosilane or [tris(trimethylsiloxy)silylethyl]dimethylchlorosilane produces a hydrophobic surface that facilitates the robust sealing of PDMS microreactors. Also, this hydrophobic and/or fluorinated surface can be passivated effectively with nonionic detergents. Finally treatment of the surface with bi-functional reactive silanes, such as 3-mercaptopropyltrimethoxysilane (Liu et al. Langmuir, 2004, 20(14), 5905-5910), allows for direct, covalent coupling of protein, DNA, or other molecules such as biotin to the glass surface.

### Example 2

In order to immobilize a population of substantially identical nucleic acids in the microreactors, we developed a method to pattern biotin spatially within the microreactor. First, 5 micron diameter microreactors were generated using previously described photolithographic methods (see Example 1). The PDMS was then exposed to air plasma for 1 minute, hydrochloric acid vapor for 10 seconds, then 3-mercaptopropyltrimethoxysilane (Gelest) in vapor phase under vacuum at 40° C. for 10 minutes (Liu et al. Langmuir, 2004, 20(14), 5905-5910). Following this, 0.5 mg/mL maleimidophenyl PEG LC biotin (Apollo Scientific) in phosphate buffer pH 7.5 was introduced to a region between the PDMS and a coverslip previously treated with 1H, 1H, 2H, 2H-perfluorooctyltrichlorosilane in the vapor phase under vacuum. The PDMS microreactors were quickly sealed to the coverslip, and the maleimidophenyl PEG LC biotin solution was allowed to react for 30 minutes. The reactants were then washed in water, and dried. Then, the entire surface of the PDMS was immersed in 10 mg/mL methoxypolyethylene glycol maleimide (MW 5,000, Sigma) in phosphate buffer. Finally the PDMS was treated to 1H, 1H, 2H, 2H-perfluorooctyltrichlorosilane for 20 minutes in vapor phase at room temperature under vacuum in order to make the PDMS hydrophobic. Finally, streptavidin coated beads were allowed to bind to the surface. The beads were incubated for a period such that the density was more than one bead per hole on average, in order to demonstrate the robust patterning of the interior of the holes (FIG. 18).

### Example 3

In this example, 500 nm streptavidin-coated polystyrene beads (Bangs Laboratories) were incubated at a concentration of 50 μM for 20 minutes in reaction buffer (50 mM Tris-HCl pH 8, 50 mM NaCl, 0.1% Tween-20, 0.2% Pluronic-F108, 1% PEG-10K) with 5 nM biotinylated template DNA (a primed poly-C homopolymer) on ice. The composition of the reaction mixture was then adjusted to include dGTP-γ-resorufin (20 μM), MnCl₂ (1 mM), SAP (1 μM), and either φ29 (exo-) DNA polymerase or Klenow fragment (exo-) DNA polymerase on ice. When Klenow fragment (exo-) DNA polymerase was used, 0.25 mM DTT was included in the reaction mixture. The reaction mixture was immediately scaled in passivated, non-biotinylated PDMS microreactors (either 5 μm or 1.5 μm in diameter) and imaged on a fluorescence microscope.

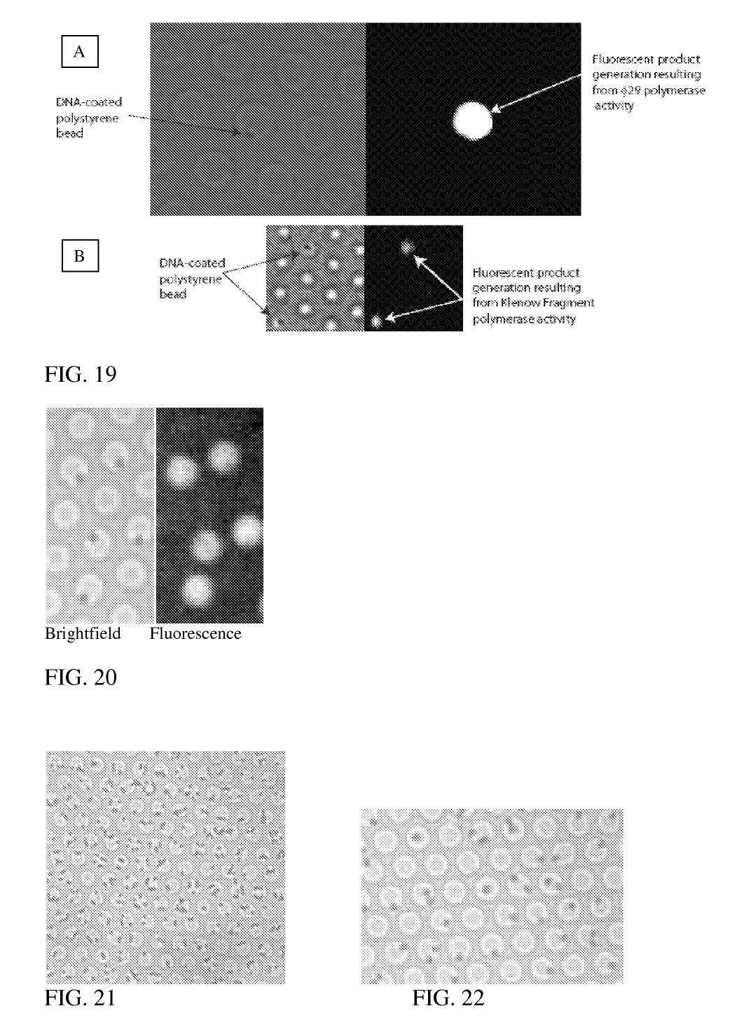

A microscope (Nikon TE-2000 with 60×1.2NA water-immersion objective) was operated in wide-field fluorescence mode with 560 nm laser excitation. Bright field and fluorescence signals were imaged onto an EM-CCD camera (Cascade 512B, Roper Scientific). The resulting images are shown in FIGS. 19A-19B.

### Example 4

In a separate experiment, we incubated 30 μM, 1 μm streptavidin coated beads (Bangs Labs) with 300 nM biotinylated DNA consisting of a primed oligonucleotide with a single G base as then next base to be incorporated. These beads were then incubated at 3 μM concentration in PDMS microreactors that were 5 microns in diameter. This microreactor device had been previously patterned such that the interior of the microreactors contained covalently attached biotin (see Example 2). Therefore, the beads slowly diffused into the reactors and irreversibly bound. When these beads occupied approximately half the reactors, the excess beads in the bulk were washed away and the reactors were sealed to a PEGylated glass surface that had also been treated with 1H, 1H, 2H, 2H-perfluorooctyltrichlorosilane for 35 minutes in vapor phase under vacuum in order to make the surface hydrophobic. Then a reaction mixture consisting of 50 mM Tris-HCl (pH 7.5), 50 mM NaCl, 1 mM MnCl₂, 1 mM DTT, 1 μM dG4P-3′-O-methyl-fluorescein-5(6)-carboxylic acid substrate, 0.0125 U/μL SAP, and 0.25 U/μL Klenow fragment (exo-) DNA polymerase (NEB) was introduced around the sealed region of the reactors. Then the reactors were opened for ˜5 seconds, allowing the reaction mixture to replace the wash buffer by flow and diffusion, and then resealed. After 2 minutes, a fluorescence image, taken using 0.05 kW/cm², 476 nm widefield illumination with an EM-CCD camera (Cascade 512B, Roper Scientific), shows clear buildup of signal in only holes that contained beads with immobilized DNA (FIG. 20).

### Example 5

Similarly to Example 2, selective spatial exposure to oxygen plasma was used to pattern biotin on 5 μm microreactors made in PDMS. In brief, the PDMS reactors were obtained from a master generated using photolithography as described in Example 1. These PDMS holes were then sealed in air to a clean glass slide, trapping air within the microreactors. The sealed holes were then placed into a plasma cleaner (Harrick) and exposed to air plasma in vacuum for 1 minute, which selectively exposed only the interior of the microreactors to air plasma. Upon removal of the PDMS from the glass, the PDMS was exposed to HCl vapor for 10 seconds and then exposed to 3-mercaptopropyltrimethoxysilane (Gelest) under vacuum at 40° C. for 10 minutes (Liu et al. Langmuir, 2004, 20(14), 5905-5910). Following this, 0.5 mg/mL maleimidophenyl PEG LC biotin (Apollo Scientific) in phosphate buffered saline pH 7.5 was placed on top of the microreactors for 30 minutes, and then they were washed with water. Finally, a 30 pM solution of 1 μm streptavidin-coated polystyrene beads was incubated on these microrcactors for approximately 2 minutes, the reactors were washed with water, and then sealed to glass to determine the quality of the patterning, shown in FIG. 21. In addition, polystyrene beads that are not coated in streptavidin do not specifically bind to the microrcactors which have been treated as described above. Repeating this experiment without treatment of the PDMS to maleimidophenyl PEG LC biotin does not generate specifically adsorbed streptavidin-coated beads within the microreactors.

### Example 6

Using 5-micron diameter microreactors that were patterned using oxygen plasma as described in Example 6, we immobilized 1 μm diameter streptavidin-coated superparamagnetic beads (Dynabeads® MyOne Streptavidin Cl, Invitrogen) to the reactors as follows. First, the beads were washed in 50 mM Tris-IICl pH 8.0, 50 mM NaCl, 1 mg/mL Tween-20, and 2 mg/mL Pluronic F-108, 10 mg/mL PEG (MW 10 k) and incubated with ˜3000 copies of primer/template DNA per magnetic bead. These beads were then diluted to a concentration such that there was approximately 1 bead for every microreactor in 5 fL of the wash buffer. The microreactor array was placed on a strong magnet (in order to pull the paramagnetic beads into the holes), and a bead solution was placed on the microreactor array. After 2 minutes, the reactors were inspected and found to be well patterned with an average density, in regions, of approximately 0.7 beads per microreactor (see FIG. 22).

### Example 7

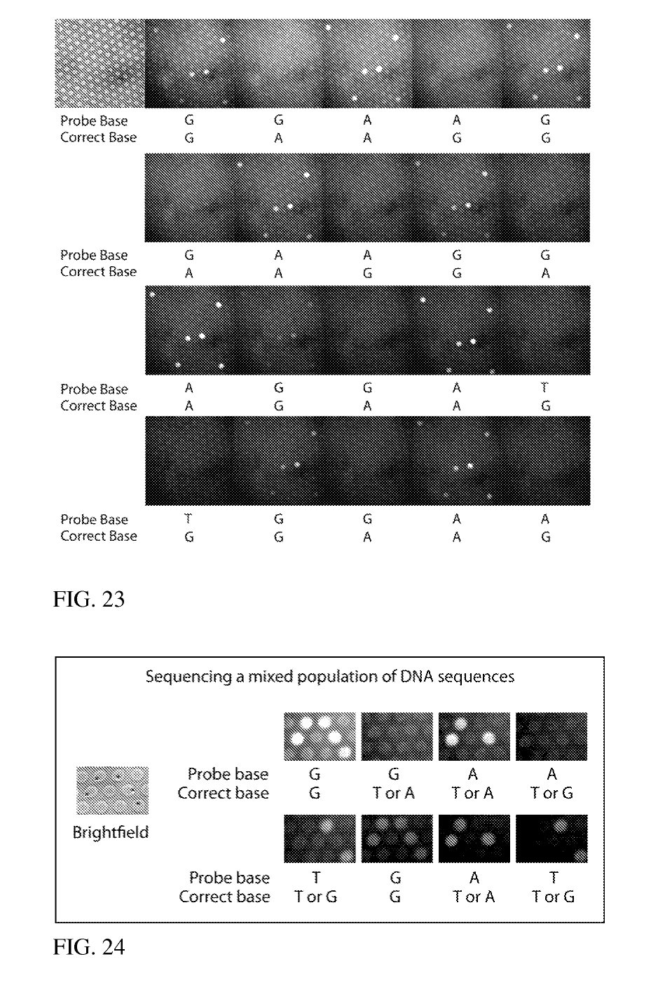

This example demonstrates a 10 base DNA sequencing read on an alternating template. Streptavidin-coated, 1 micron diameter polystryene beads (Bangs Labs) were incubated with 10,000 copies per bead of a self-primed hairpin poly-CT template with dual 5′ biotins for immobilization. These beads were then immobilized in biotin-coated (through plasma-patterning), 5 micron diameter microreactors. These reactors were then probed by different reaction mixtures (50 mM Tris-IICl (pH 8.0), 50 mM NaCl, 1 mM MnCl₂, 1 mM DTT, 10 mg/mL polyethylene glycol-10000, 2 mg/mL Pluronic F-108, 1 mg/mL Tween-20, 0.0125 U/μL SAP and 0.25 U/μL Klenow fragment (exo-) DNA polymerase (NEB)) with 1.5 μM of either dG4P-3′-O-methyl-5(6)-carboxyfluorescein, dA4P-3′-O-methyl-5(6)-carboxyfluorescein, or dT4P-3′-O-methyl-5(6)-carboxyfluorescein. Signal was generated in microreactors when the complementary nucleotide substrate was added, as shown in FIG. 23.

Fluorogenic sequencing with a mixed population of DNA sequences was also demonstrated with Klenow fragment (exo-). Two different populations of 1 micron diameter streptavidin-coated polystyrene beads (Bangs Labs) were prepared, each with a different self-primed hairpin template with dual 5′ biotins for immobilization to the bead. For one population of beads, 10,000 copies of a poly-CT repeat template were immobilized to each bead. For the other population of beads, 10,000 copies of a poly-CA repeat template were immobilized to each bead. These beads were then mixed in equimolar ratio and immobilized in biotin coated (through plasma-patterning), 5 micron diameter microreactors. All beads initially generate signal upon exposure to 1.5 μM dG4P-3′-O-methyl-5(6)-carboxyfluorescein in reaction buffer (50 mM Tris-HCl (pH 7.5), 50 mM NaCl, 1 mM MnCl₂, 1 mM DTT, 10 mg/mL polyethylene glycol-10000, 2 mg/mL Pluronic F-108, 1 mg/mL Tween-20, 0.0125 U/μL SAP and 0.25 U/μL Klenow fragment (exo-) DNA polymerase (NEB)) because all beads have DNA that code for C in the template. Subsequent exposure to dA4P-3′-O-methyl-5(6)-carboxyfluorescein (1.5 μM in reaction buffer) generated signal from only the population of beads which code for a T in the template strand (i.e. the population of beads with a poly-CT template). Further exposure to dT4P-3′-O-methyl-5(6)-carboxyfluorescein (1.5 μM in reaction buffer) generated signal from the other half of the beads which did not generate signal upon addition of dA4P-3′-O-methyl-5(6)-carboxyfluorescein (i.e., the population of beads with a poly-CA template). Finally, subsequent serial additions of reaction buffer containing dG4P-3′-O-methyl-5(6)-carboxyfluorescein, dA4P-3′-O-methyl-5(6)-carboxyfluorescein, and dT4P-3′-O-methyl-5(6)-carboxyfluorescein, generated signal in all the holes, then in holes containing beads with the poly-CT template, then in holes with beads containing the poly-CA template respectively. These results are shown in FIG. 24

### Example 8

Microreactor Array Preparation.

A PDMS microreactor array containing 5 μm holes was fabricated from a silicon master array of 5 μm pillars (in SU-8 photoresist) by pouring Sylgard 184 (10:1 PDMS base to curing agent ratio) on the silicon master and curing overnight at 70° C. The PDMS microreactor array was peeled from the master and sealed to a glass slide, trapping air in the microreactors. The microreactors sealed with the glass slide were treated with air plasma for 60 seconds in a plasma sterilizer and then removed from the glass slide. About 100 μL of 0.1% aminotriethoxysilane (APTES) in ethanol was applied to the microreactor array and incubated at room temperature for 10 minutes. The microreactor array was rinsed with MilliQ water and dried with nitrogen. NHS-PEG4-biotin (Pierce) was dissolved in 100 mM sodium bicarbonate buffer (pH 8.5) at about 1 mg/mL. About 100 μL of this solution was then applied to the microreactor array, which was then placed under vacuum for 3 minutes to wet the microreactors. The solution was then incubated on the microreactor array for 3 hours at room temperature. The microreactor array was then rinsed with MilliQ water and dried with nitrogen. This procedure results in a PDMS microreactor array, where the inner walls of each microreactor are biotinylated, but the interstitial regions are not.

Microfluidic Device Preparation.

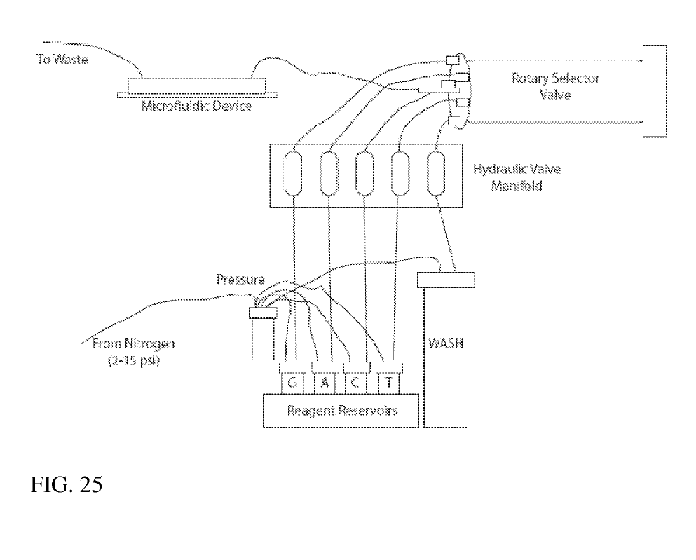

A 15 μm coating of Sylgard 184 (10:1 PDMS base to curing agent ratio) was spun onto a glass coverslip and cured overnight at 70° C. In addition, a single microfluidic channel (500×50 μm cross section) was also fabricated from PDMS. A hole was cut in the top of the channel allowing the upper surface of the channel to be replaced at one location with the biotinylated, PDMS microreactor array. The microfluidic device was then connected to a 6-position/7-port selector valve (Rheodyne), which was connected to a hydraulic valve manifold (The Lee Company) so that the different nucleotide reaction mixtures and wash solutions could be flowed into the device individually. This device is shown schematically in FIG. 25.

DNA Sequencing.

In all DNA sequencing experiments, streptavidin-coated beads were coated with 1,000-10,000 copies of a primed, template DNA molecule. Polystyrene, streptavidin-coated 1 μm beads (Bang's Labs) were washed three times in binding buffer (50 mM Tris-HCl pH 8.0, 1 M NaCl, 0.1% Tween-20) and incubated for 60 minutes at room temperature with the appropriate concentration of biotinylated DNA. The beads were then introduced to the microfluidic device and incubated for 5 minutes so that a portion of the beads bound to the microreactors.

A LabVIEW program was used to control a fluidics module (including the selector valve and hydraulic valve manifold), an imaging module (including a Cascade 512B EM-CCD camera from Roper Scientific and an electronic shutter from Uniblitz), and a sealing module (Oriel stepper motor used to press a glass tube against the microreactor array to seal the microreactors against the lower PDMS surface of the device). Imaging was carried out on a Nikon TE-2000 Eclipse with an Olympus 50×, 0.75 NA M-PLAN objective. Illumination was provided by a diffused 476 nm laser beam from an Innova 300 FRED argon ion laser (Coherent). Reaction mixtures, each of which contained a single fluorogenic nucleotide, were introduced to the microfluidic device sequentially with a washing step between each cycle. The four reaction mixtures had the following composition:
1) Reaction Buffer, 1 μM dG4P-δ-3′-O-methylfluorescein-5(6)-carboxylic acid, 10 nM Klenow fragment exo- (New England Biolabs), 10 nM shrimp alkaline phosphatase (United States Biochemical)
2) Reaction Buffer, 1.5 μM dA4P-5-3′-O-methylfluorescein-5(6)-carboxylic acid, 10 nM Klenow fragment exo- (New England Biolabs), 10 nM shrimp alkaline phosphatase (United States Biochemical)
3) Reaction Buffer, 1 μM dC4P-δ-3′-O-methylfluorescein-5(6)-carboxylic acid, 10 nM Klenow fragment exo- (New England Biolabs), 10 nM shrimp alkaline phosphatase (United States Biochemical)
4) Reaction Buffer, 1.5 μM dT4P-δ-3′-O-methylfluorescein-5(6)-carboxylic acid, 10 nM Klenow fragment exo- (New England Biolabs), 10 nM shrimp alkaline phosphatase (United States Biochemical)
   The reaction buffer was 50 mM Tris-HCl pH 8.0, 50 mM NaCl, 1 mM MnCl₂, 1 mM DTT, and 0.1% Tween-20. This buffer also served as the wash buffer that was introduced between cycles. Each nucleotide reaction mixture was introduced to the device with the microreactor array sealed. The array was then quickly unsealed and resealed to initiate the reaction. After about one minute, the array was imaged with bright field illumination to autofocus the array using a piezo stage (Physik Instrumente) and a feedback algorithm. Once the array was in focus, a fluorescence image was acquired (500 ms exposure, 0.04 kW/cm²).

In one experiment, a DNA template was designed to test the system's ability to sequence homopolymers. The DNA template had the following sequence:

Template:
(SEQ ID NO: 1)
CTCTTCTTTCTTTTCTTTTTG
Complement:
(SEQ ID NO: 2)
GAGAAGAAAGAAAAGAAAAAC

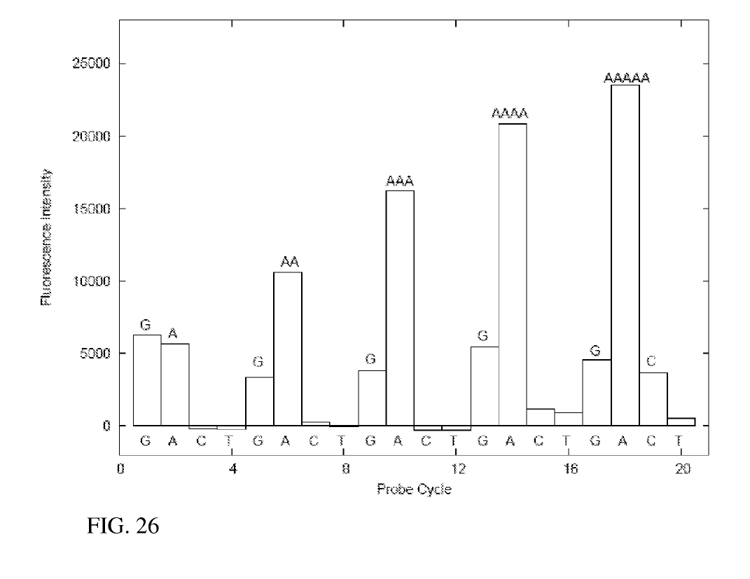

The fluorescence intensity (after background subtraction) in a bead-containing microreactor for each probe cycle was obtained from the series of images resulting from this sequencing experiment. FIG. 26 shows the results of the sequencing. Fluorescence intensity (after background subtraction) for each sequencing probe cycle corresponding to a microreactor containing a homopolymeric DNA template was obtained. The fluorescence intensity was proportional to the length of the homopolymer. Little or no signal was observed in probe cycles that do not correspond to the correct base in the template.

In a second experiment, a random DNA template was chosen with following sequence:

Template:
(SEQ ID NO: 3)
TGCGGTCTTTGGCGG
Complement:
(SEQ ID NO: 4)
ACGCCAGAAACCGCC

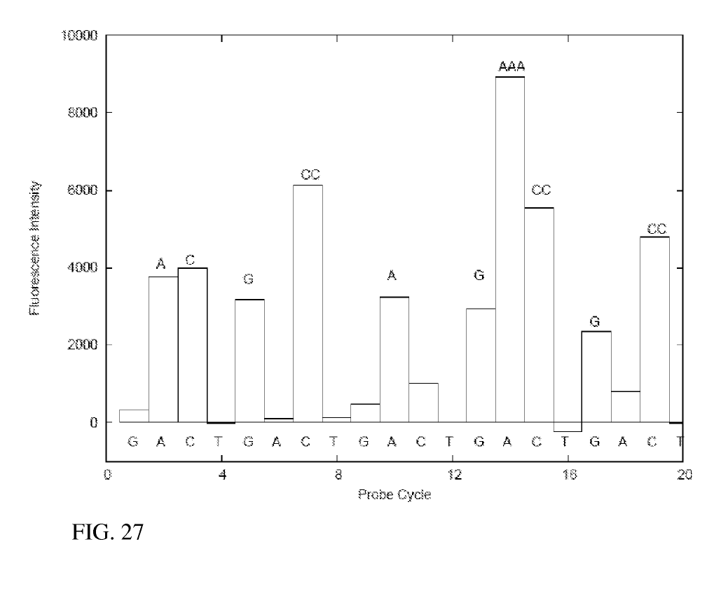

The fluorescence intensity (after background subtraction) in a bead-containing microreactor for each probe cycle was obtained from the series of images resulting from this sequencing experiment, as shown in FIG. 27. Fluorescence intensity (after background subtraction) for each sequencing probe cycle corresponding to a microreactor containing a random DNA template was obtained. The fluorescence intensity was proportional to the length of homopolymeric sequences in the template. Little or no signal was observed in probe cycles that do not correspond to the correct base in the template.

### Example 9

We developed a method to pattern biotin spatially with the microreactor to allow immobilization of a population of substantially identical nucleic acids in an array of microreactors. Dome-Shaped PDMS microreactors with a diameter of about 5 μm were generated using previously described photolithographic methods (see Example 1). The PDMS microreactor array was then sealed in air to a clean glass slide, trapping air within the microreactors. The sealed microreactors were then placed into a plasma cleaner (Harrick) and exposed to air plasma for 1 minute, which selectively exposed only the interior of the microreactors. Upon removal of the PDMS from the glass, the PDMS was exposed to HCl vapor for 10 seconds and then exposed to 3-mercaptopropyltrimethoxysilane (Gelest) under vacuum at 40° C. for 10 minutes (Liu et al., Langmuir, 2004, 20(14), 5905-5910). Following this step, 10 mg/mL maleimide-PEG-biotin (Laysan) in phosphate buffered saline pH 7.5 was placed on top of the microreactors. The microreactors were placed under vacuum briefly to ensure that the solution wetted the inside of the microreactors. The reaction was incubated at room temperature for 2 hours after which the microreactors were washed thoroughly with water and dried with nitrogen.

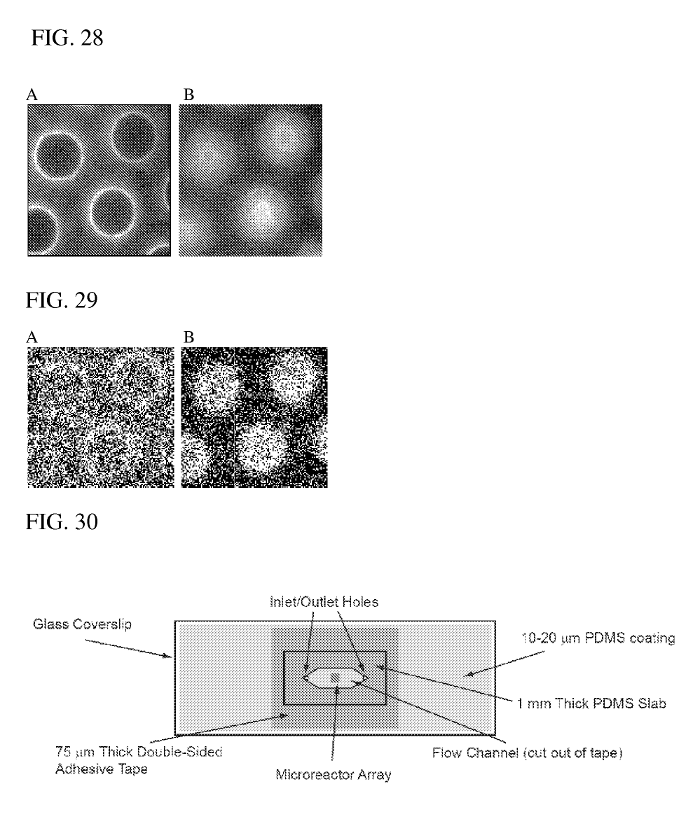

To demonstrate the surface patterning, streptavidin labeled with AlexaFluor-488 (Invitrogen) was incubated with the microreactor surface briefly at a concentration of 0.02 mg/mL in high salt buffer (50 mM Tris-HCl pH 8, 1 M NaCl, 0.1% Tween-20). After thoroughly washing the surface with high salt buffer, the microreactor array was placed face-down on a glass coverslip and imaged on an inverted epifluorescence microscope (Nikon TE-300). 470 nm light from an LED (Thorlabs) was delivered to the sample with a 60×, 1.4 NA oil-immersion objective (Nikon), and fluorescence emission was collected with the same objective and imaged onto a CCD camera (Cool Snap, Roper Scientific). FIGS. 28A-B show images taken at two different focal planes in the same microreactors. FIG. 28A shows the lower surface in a plane level with the opening of the microreactors. FIG. 28B shows the upper surface of the dome-shaped microreactor where the fluorescence signal was collected from the labeled top of the microreactor. The fluorescently labeled streptavidin was clearly patterned on the inner walls of the PDMS microreactors, indicating that the covalently attached biotin was as well.

To demonstrate the surface patterning and surface capture of DNA, unlabeled streptavidin (Invitrogen) was incubated with the microreactor surface briefly at a concentration of 0.02 mg/mL in high salt buffer. The surface was then washed thoroughly with high salt buffer and incubated with a 40-base ssDNA oligo dual-labeled with biotin on its 5′ end at a concentration of 1 μM. After washing the surface with high salt buffer, the surface was incubated with the complementary 40-base ssDNA oligo fluorescently labeled with FAM on its 5′ end at a concentration of 1 μM. After thoroughly washing the surface with high salt buffer, the microreactor array was imaged using the same fluorescence microscope described above. Although the labeling density was, as expected, somewhat lower than in the labeled streptavidin experiment, the same patterned immobilization of fluorophores is observed demonstrated patterned oligonucleotide capture in PDMS microreactors (FIGS. 29A-B).

### Example 10

A 40-base ssDNA primer dual-labeled with biotin on its 5′ end (Integrated DNA Technologies) with the sequence:

(SEQ ID NO: 5)
5′-CCTATCCCTGTGTGCCTGCCTATCCGTTGCGTGTCTCAG-3′

was incubated with 1 μm streptavidin-coated polystyrene beads (Bang's Labs) for one hour at room temperature at a 10,000:1 molar ratio (300 nM DNA, 30 μM beads) in High Salt Buffer:

### 50 mM Tris-HCl pH 8.0

### 1 M NaCl

### 0.1% Tween-20

The beads were then washed three times in Annealing Buffer:

### 50 mM Tris-HCl pH 8.0

### 50 mM NaCl

### 1 mM EDTA

### 0.1% Tween-20

by centrifugation at 5800×g for 2.5 min. The beads were then incubated for 2 min at 65° C. with a ssDNA template (3 μM; Integrated DNA Technologies) with the sequence:

(SEQ ID NO: 6)
5′-TGTATCACTATGACGCGCCTGACTCTCTGACTGAGACACGCAACG
GATAGGCAGGCACACAGGGATAGG-3′

and slowly cooled to room temperature over the course of one hour. The beads were then washed three times in Annealing Buffer and one time in High Salt Buffer.

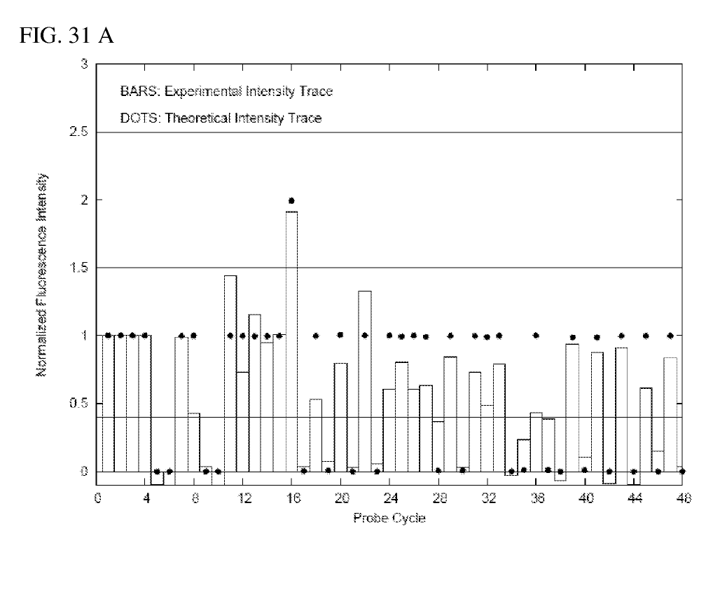

A flow cell was created out of a PDMS-coated glass coverslip, a double-sided adhesive tape spacer with a chamber cut out of the center, and a PDMS slab containing an array of ˜100,000 hexagonally close-packed 5 μm microreactors (FIG. 30). The inner walls of the microreactors were patterned with biotin as described in Example 11. Both the PDMS coated coverslip and PDMS slab were oxidized in a plasma cleaner (Harrick) everywhere except the area of the PDMS-coated coverslip to which the array seals and the microreactor array itself. This ensures that the array area is hydrophobic (for high fidelity sealing) while the remainder of the chamber is hydrophilic. Two holes were punched on the two ends of the chamber to allow fluids to flow across the microreactor array. About 10 μL of High Salt Buffer was introduced to the flow cell and incubated for 15 minutes followed by the introduction of primed DNA template-coated beads. Because the beads have many free streptavidins on their surface, they are selectively immobilized in the PDMS microreactors. The incubation takes place at a concentration and for a duration that allows the microreactors to have zero, one, or two beads immobilized on their inner walls.

A LabVIEW/C/C++ program controls the mechanical sealing and imaging of the PDMS microreactor array as well as fluidic flow. A stepper motor is used to move a glass tube up and down to rapidly seal and unseal the microreactor array. Fluid flow is controlled by an array of hydraulic valves (The Lee Company) and a rotary selector valve (Rheodyne). Bright field imaging of the microreactors is used to provide focus feedback with the z-axis of a piezo stage (Mad City). Epifluorescence imaging is accomplished by exciting the sample with 0.1 kW/cm² of 476 nm laser light from an Argon laser (Coherent) which is diffused to provide homogeneous illumination of the sample. Fluorescence is collected with a 50×0.75 NA air objective (Olympus) and imaged onto an EM-CCD camera (Cascade 512B, Roper Scientific).

Each probe cycle in the sequencing run involves first introducing a DNA polymerase-containing solution:

### 50 mM Tris-HCl pH 8.0

### 50 mM NaCl

### 1 mM DTT

### 0.1% Tween-20

9 nM Klenow fragment (exo-) (New England Biolabs)
and incubating it with the unsealed microreactors for 30 s. The microreactors are then sealed, and a reaction mixture containing a single fluorogenic nucleotide is introduced to the device, which is rapidly unsealed and resealed to trigger primer extension:

### 50 mM Tris-HCl pH 8.0

### 50 mM NaCl

### 1 mM DTT

### 1 mM MnCl₂

0.1% tween-20
1.5 μM dN4P-d-3′-O-methylfluorescein-5(6)-carboxylic acid
9 nM Klenow fragment (exo-) (New England Biolabs)
0.0075 units/mL Shrimp Alkaline Phosphatase (USB)
After 1-2 minutes (depending on the nucleotide) the array is imaged; a second flow of the same nucleotide reaction mixture is introduced; and the device is rapidly unsealed and resealed followed by a second incubation and image acquisition. The device is then washed for 5 minutes with Wash Buffer at 0.75 mL/min:

### 50 mM Tris-HCl pH 8.0

### 50 mM NaCl

### 1 mM DTT

### 0.1 mM EDTA

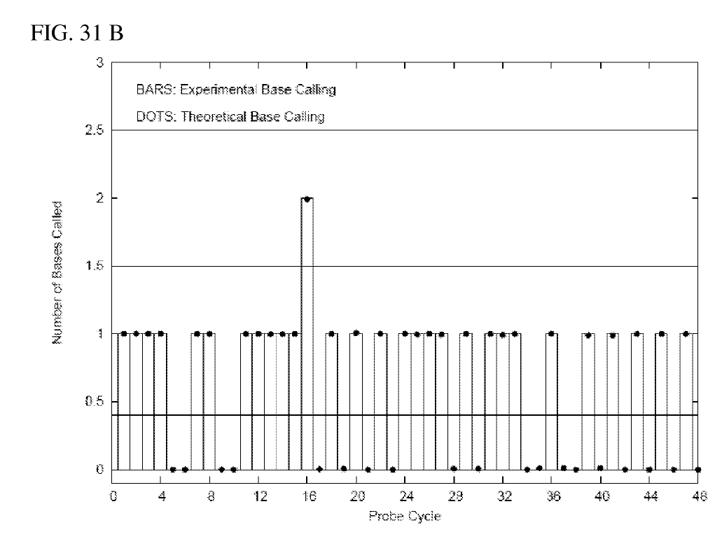

0.1% tween-20
This cycle is repeated for all four nucleotides to build up an intensity trajectory from which the DNA sequence can be extracted. In this instance, all four nucleotides were cycled through the device 12 times in a known order (TCAG), and a 30-base read was obtained. The integrated fluorescence signal from a single microreactor was computed for each nucleotide probe cycle after background subtraction and was normalized by the single base signals for G, A, T, and C, which are calibrated by the first four bases of the template (which are TCAG). For example, the computed intensities for all nucleotide probe cycles in which G is the probe base are divided by the signal obtained for the first incorporation of a single G. This accounts for kinetic heterogeneity among the four bases that may lead to differential signal loss during the sealing time. The resulting intensity trace is shown in FIG. 31A. The horizontal lines represent intensity thresholds for single, double, and triple base incorporations (0.4, 1.5, and 2.5 respectively). Based on the intensity thresholding, we can compute the number of bases incorporated in each cycle and obtain the DNA sequence, as shown in FIG. 31B.

### Example 11

In most cases, silicon masters are used repeatedly to generate PDMS devices using soft lithography. The repeated use of a PDMS master that is derived once from a silicon master has a number of advantages for mass-producing PDMS microreactor arrays:
1) Direct peeling of PDMS-coated coverslips (with PDMS layers that are <10 microns thick) from silicon masters causes plastic deformation of PDMS sheets, complicating the fabrication of uniformly flat microreactor arrays for imaging and sealing. However, a flexible, elastomeric PDMS master containing a micropillar array can be removed from a PDMS-coated coverslip without bending the coverslip.
2) Repeated use of PDMS masters is more economical than repeated use of silicon masters.

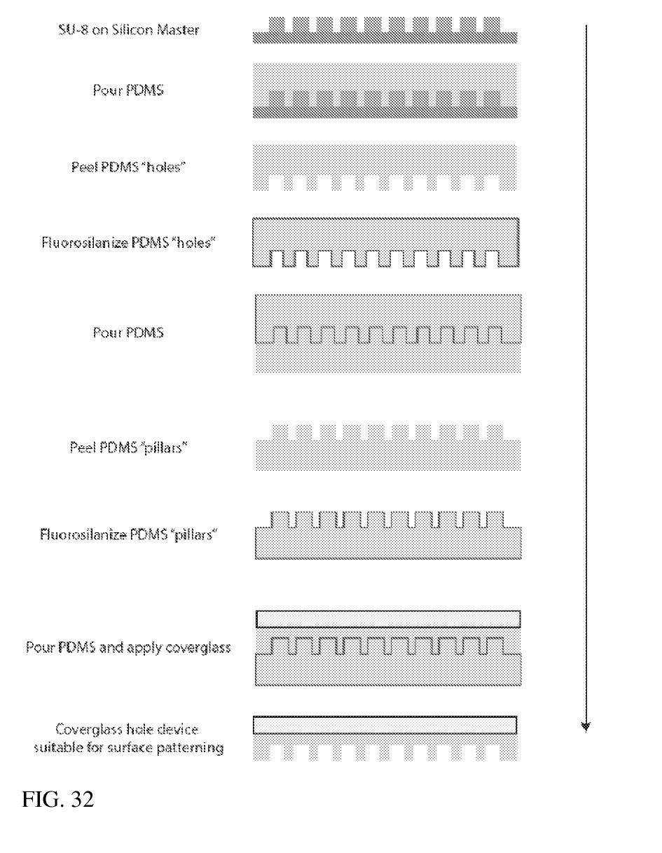

PDMS micropillar masters can be fabricated from silicon micropillar arrays by first curing PDMS onto a silicon micropillar array master, peeling it, and fluorosilanizing the resultant PDMS microreactor array with 1H, 1H, 2H, 2H-perfluorooctyltrichlorosilane by chemical vapor deposition. PDMS can then be cured onto the fluorosilanized PDMS microreactor array to generate a PDMS micropillar array which can, in turn, be fluorosilanized and used as a master. To generate PDMS microreactor arrays in ultra-thin PDMS layers mounted on glass coverslips, a ˜5-10 micron thick layer of PDMS is spin-coated onto a No. 1.5 glass coverslip, and the fluorosilanized PDMS micropillar array master is placed face down on an uncured PDMS layer. This object is then cured, and the PDMS micropillar array master is peeled from the coverslip, generating a coverslip-mounted microreactor array (FIG. 32). Alternatively, a PDMS master can be cast directly from a silicon master having the inverse pattern.

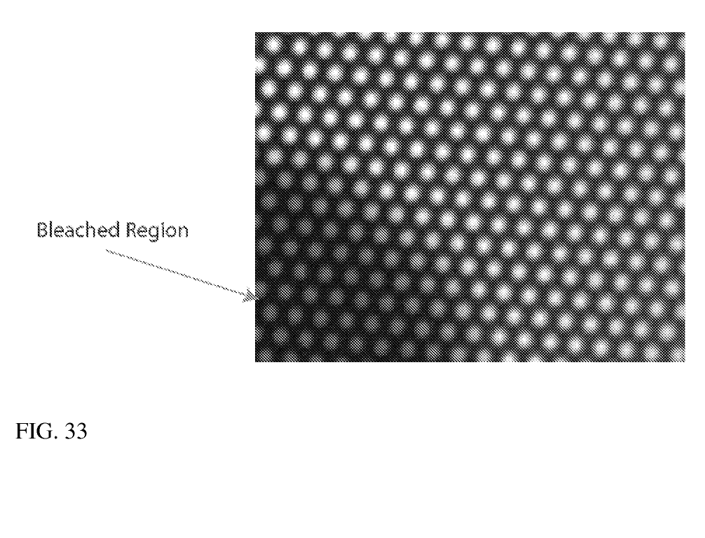

The resultant PDMS microreactor arrays can be sealed exceptionally well. One can easily photobleach an essentially permanent hole in the fluorescence image of a sealed, fluorophore-filled microreactor array fabricated using the above procedure (FIG. 33).

### Example 12

On-chip amplification is a highly efficient, inexpensive, and convenient means of producing a clonal population of copies for a target DNA template. By capturing single DNA templates immobilized on beads with surface-immobilized primers in PDMS microreactors, super-Poisson loading of a microreactor array for amplification and sequencing is achievable. We have demonstrated on-chip PCR using an end-point Taqman assay, a PDMS microfluidic device optimized to minimize sample evaporation, a Peltier-based thermocycler, and an epifluorescence microscope. In this experiment, the buffer conditions were as follows:

### 1× Taq Master Mix (New England Biolabs)

10 mM Tris-HCl pH 8.6

50 mM KCl

1.5 min MgCl₂

0.2 mM dNTPs

5% glycerol

0.08% NP-40

0.05% Tween-20

25 units/mL Taq DNA polymerases

### 0.5% Pluronic F-27

0.1 mg/mL bovine serum albumin (BSA)
500 nM forward PCR primer (Integrated DNA Technologies)
500 nM reverse PCR primer (Integrated DNA Technologies)
20 nM target DNA template (Integrated DNA Technologies)
240 nM Taqman FAM/Zen-labeled Taqman probe DNA (Integrated DNA Technologies)
2.4 units/mL thermostable inorganic pyrophosphatase (New England Biolabs)

Forward Primer:
(SEQ ID NO: 7)
5′ -CCA TCT CAT CCC TGC GTG TC- 3′
Reverse Primer:
(SEQ ID NO: 8)
5′ -CCT ATC CCC TGT GTG CCT TG- 3′
Taqman Probe:
(SEQ ID NO: 9)
5′ -TGT AGT CGC CAT GTA ACT CAT CGG CA- 3′
Template:
(SEQ ID NO: 10)
5′ -CCA TCT CAT CCC TGC GTG TCC CAT CTG TTC CCT
CCC TGT CTC AGT GTC ATT GAT GTA GTC GCC ATG TAA
CTC ATC GGC AAT AGG CTG TAA ATC CAC ATG TAC GAC
AAT CCG CGT CAG TTT ACC GCT TAA CAT ATC GAA GAA
CGG CTG AGA CAC GCA ACA GGG GAT AGG CAA GGC ACA
CAG GGG ATA GG- 3′

A PDMS microfluidic device having a flow layer with a microreactor array-containing, PDMS-coated coverslip which can be sealed with an upper PDMS membrane by water pressure from a control layer was constructed using standard photolithography and PDMS soft lithography (FIG. 2B). The device was placed in thermal contact with a metal plate mounted on a Peltier thermocycler. Both the control layer and the flow layer were then filled with water, and the control layer was pressurized at 20 psi, causing a thin membrane to seal the microreactor array at the bottom of the flow layer. Once the microreactor array was sealed, the water in the flow layer that was not trapped in the microreactor array was further pressurized at 10 psi. The device was then raised to 92° C. to saturate the PDMS with water. After 10 minutes, the device was cooled to room temperature and the above reaction mixture excluding the DNA components (e.g. primer, probe, template) was introduced to the flow layer which was then re-scaled and re-pressurized. The device was then thermocycled for 30 cycles each consisting of:

### 15 s at 92° C.

### 30 s at 58° C.

### 30 s at 68° C.

No signal was generated by the Taqman probe (FIG. 34A).

The device was then returned to room temperature, and the complete reaction mixture including all DNA components was introduced to the flow layer which was resealed and re-pressurized. The device was then thermocycled for 30 cycles using the same cycling protocol described above. After thermocycling, the device was cooled to room temperature, and a fluorescence image of the microreactor array was acquired. The microreactor array was imaged on an epifluorescence microscope (Nikon TE-300) with a 60×1.4 NA oil-immersion objective (Nikon), a 470 nm LED (Thorlabs), and a CCD camera (CoolSnap, Photometrics). Signal generation from the Taqman probe is clearly visible in a subset of the microreactors (FIG. 34B). Under the conditions of this experiment, the initial template DNA concentration is sufficiently low that only a few microreactors contain PCR products. Most of the microreactors contain zero, one, or two DNA templates due to Poisson loading.

### Example 13

A 40-base ssDNA primer dual-labeled with biotin on its 5′ end (Integrated DNA Technologies) with the sequence:

(SEQ ID NO: 11)
5′-CCTATCCCTGTGTGCCTGCCTATCCGTTGCGTGTCTCAG-3′

was incubated with 1 μm streptavidin-coated polystyrene beads (Bang's Labs) for one hour at room temperature at a 10,000:1 molar ratio (300 nM DNA, 30 μM beads) in High Salt Buffer:

### 50 mM Tris-HCl pH 8.0

### 1 M NaCl

### 0.1% Tween-20

The beads were then washed three times in Annealing Buffer:

### 50 mM Tris-HCl pH 8.0

### 50 mM NaCl

### 1 mM EDTA

### 0.1% Tween-20

by centrifugation at 5800×g for 2.5 mins. The primer-coated beads were then split into three tubes, each of which was incubated for 2 hours at room temperature with a different DNA template (Integrated DNA Technologies) in order to generate three sets of beads conjugated to three different primed template DNA sequences at about 10,000 copies per bead:

Template A:
(SEQ ID NO: 12)
5′ -ATG TGT ATT AAT GAT GAG CCG CCA GGA GCA CCT
CCA TCT ATT TTT CTC GGG CCT AGC TGA CTG AGA CAC
GCA ACG GGA TAG GCA GGC ACA CAG GGA TAG G- 3′
Template B:
(SEQ ID NO: 13)
5′ -ACT ATG AGA GTG TTC CAC ACA CCG CGT TGC CCT
ACA CTC GCT GCC GAC TCA ATG GTC TGA CTG AGA CAC
GCA ACG GGA TAG GCA GGC ACA CAG GGA TAG G- 3′
Template C:
(SEQ ID NO: 14)
5′ -CCC CCT CTT CTT TCT TTT GTT TTT CTT TTC TTT
CTT CTC CTG AGA CAC GCA ACG GCA TAG GCA GGC ACA
CAG GGA TAG G- 3′

The beads were then washed three times in Annealing Buffer and once in High Salt Buffer.

A flow cell was created from a PDMS-coated glass coverslip, a double-sided adhesive tape spacer with a chamber cut out of the center, and a PDMS slab containing an array of ˜100,000 hexagonally close-packed 5 μm microreactors, e.g., as shown in FIG. 30. The inner walls of the microreactors were patterned with biotin as described above. Both the PDMS coated coverslip and PDMS slab were oxidized in a plasma cleaner (Harrick) everywhere except the area of the PDMS-coated coverslip to which the array seals and the microreactor array itself. This ensures that the array area was hydrophobic (for high fidelity sealing) while the remainder of the chamber is hydrophilic. Two holes were punched on the two ends of the chamber to allow fluids to flow across the microreactor array. About 10 μL of High Salt Buffer was introduced to the flow cell and incubated for 15 minutes followed by the introduction of primed DNA template-coated beads. Because the beads have many free streptavidins on their surface, they were selectively immobilized in the PDMS microreactors. The incubation took place at a concentration and for a duration that allows the microreactors to have zero, one, or two beads immobilized on their inner walls.

After binding the beads to the inner walls of the microreactors, the flow cell was washed with 50 volumes of Thermocycle Sequencing Wash Buffer:

### 20 mM Tris-HCl pH 8.8

### 20 mM NaCl

### 10 mM (NH₄)₂SO₄

### 0.1 mM EDTA

### 0.1% Tween-20

A LabVIEW/C/C++ program controlled the mechanical sealing and imaging of the PDMS microreactor array as well as fluidic flow and temperature control. A stepper motor was used to move a glass tube up and down to rapidly seal and unseal the microreactor array. Fluid flow was controlled by an array of hydraulic valves (The Lee Company) and a rotary selector valve (Rheodyne). Bright field imaging of the microreactors was used to provide focus feedback with a motorized focus knob. Epifluorescence imaging as accomplished by exciting the sample with 0.1 kW/cm² of 476 nm laser light from an Argon laser (Coherent), which was diffused to provide homogeneous illumination of the sample. Fluorescence as collected with a 20×0.75 NA air objective (Olympus) and imaged onto an EM-CCD camera (Cascade 512B, Roper Scientific). Temperature control was accomplished using a Peltier-based temperature controller (TE Technology).

Each probe cycle in the sequencing run involved first introducing a DNA polymerase-containing solution:

### 20 mM Tris-HCl pH 8.8

### 20 mM NaCl

### 10 mM (NH₄)₂SO₄

### 0.1% Tween-20

### 9-27 nM Bst Large Fragment DNA Polymerase (New England Biolabs)

and incubating it with the unsealed microreactors for 30 s. The microreactors were then sealed, and a Thermocycle Sequencing Reaction Mixture containing a single fluorogenic nucleotide was introduced to the device:

### 20 mM Tris-HCl pH 8.8

### 20 mM NaCl

### 10 mM (NH₄)₂SO₄

### 1 mM MnCl₂

### 0.1% Tween-20

2.0 μM dN4P-d-3′-O-methylfluorescein-5(6)-carboxylic acid
9-27 nM Bst Large Fragment DNA Polymerase (New England Biolabs) 0.0075 units/mL biotinylated alkaline phosphatase from bovine source (New England Biolabs)

The device was then cooled to 3° C. where Bst Large Fragment DNA Polymerase was ˜1000× less active than at 65° C. and ˜400-500× less active than at 25° C., and then the microreactor array was rapidly unsealed and sealed to allow the introduction of the reaction mixture to the DNA templates. Once the device was sealed, the device was heated to 62° C., triggering primer extension. After 1.5-3 minutes (depending on the nucleotide) a fluorescence image of the sealed microreactor array was acquired. The device was then washed for 2.5-5 minutes with Thermocycle Sequencing Wash Buffer at 1.0 mL/min:

### 20 mM Tris-HCl pH 8.8

### 20 mM NaCl

### 10 mM (NH₄)₂SO₄

### 0.1 mM EDTA

### 0.1% Tween-20

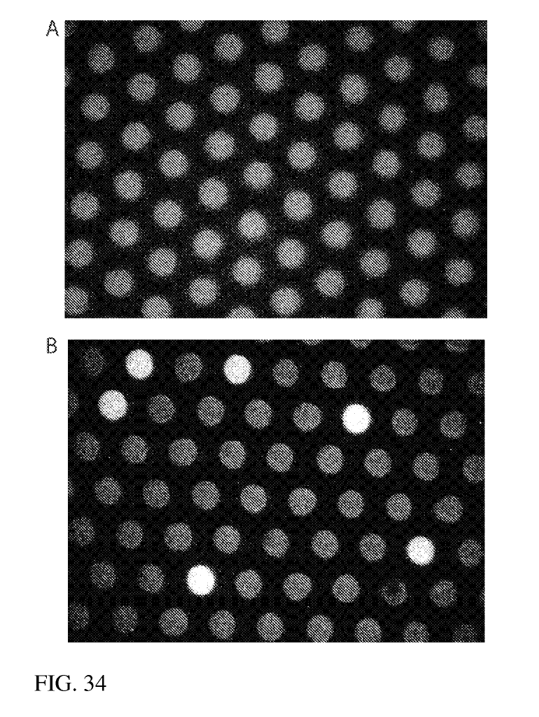

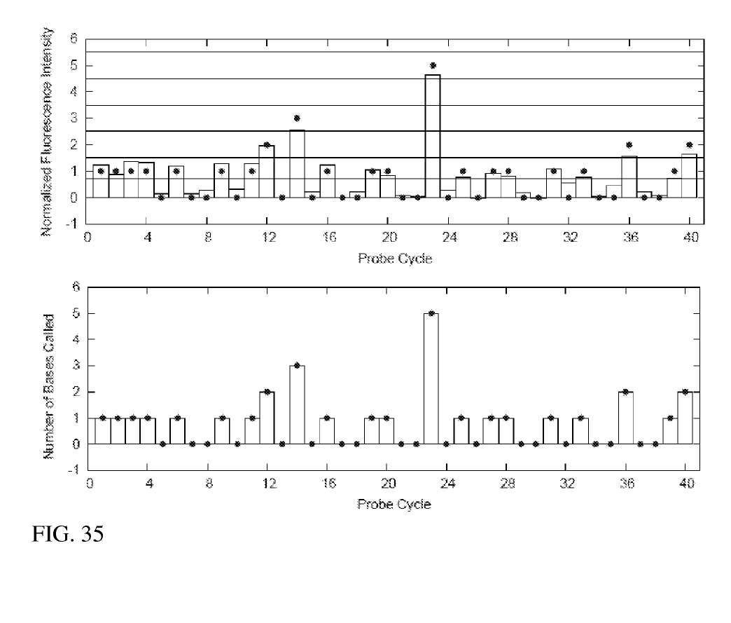

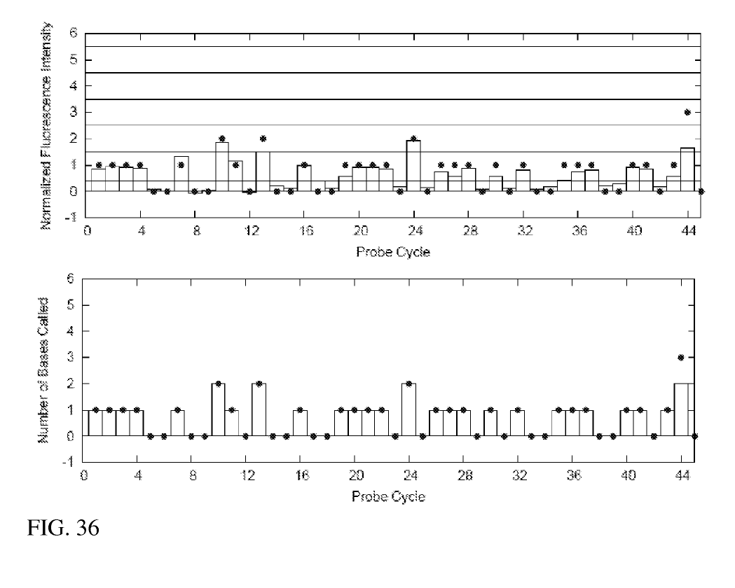

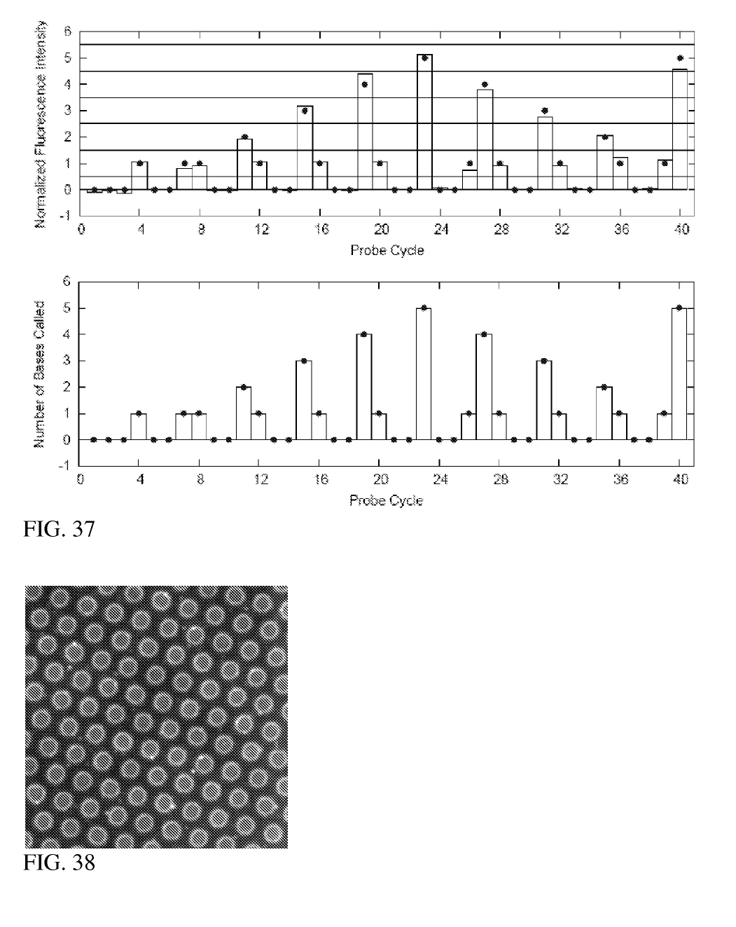

This cycle was repeated for all four nucleotides to build up intensity trajectories from which the DNA sequences were extracted. In this instance, all four nucleotides were cycled through the device 10 times in a known order (TCAG). The integrated fluorescence signal from a single microreactor was computed for each nucleotide probe cycle after background subtraction and normalized by the single base signal (FIGS. 35-37).

### Example 14

A PDMS microreactor array containing 5-μm holes was fabricated from a silicon master array of 5-μm pillars (in SU-8 photoresist) by pouring Sylgard 184 (10:1 PDMS base to curing agent ratio) on the silicon master and curing overnight at 70° C. The PDMS microreactor array was peeled from the master and sealed to a glass slide, trapping air in the microreactors. The glass slide with sealed microreactors was treated with air plasma for 60 seconds in a plasma sterilizer and then removed from the glass slide.

About 50 μL of glacial acetic acid was added to 10 mL water, and 2 mL of this dilute acetic acid solution was then added to 40 mL of ethanol (200 proof). The acidic ethanol solution was placed under nitrogen, and trimethoxysilane aldehyde (United Chemical Technologies) was added to a final concentration of 1%. The silane was incubated in acidic ethanol under nitrogen for 10 minutes at room temperature before the plasma treated PDMS microreactor array was submerged in the silane solution. The PDMS microreactor array was incubated in the silane solution under nitrogen. After one minute, the PDMS microreactor array was dipped briefly in acidic ethanol in the absence of silane before being placed face-up on a heat block at 100° C. for one minute. A 10 μM solution of 5′-aminated PCR forward primer in Cyanoborohydride Coupling Buffer (20 mM sodium phosphate pH 7.5, 200 mM sodium chloride, 3 g/L sodium cyanoborohydride, Sigma) was pipetted onto the microreactor array surface, which was placed under vacuum for 2 hours at room temperature. The microreactor array was then rinsed thoroughly with MilliQ water and dried with nitrogen.

To demonstrate covalent patterning of aminated primer on the inner walls of PDMS microreactors, a microreactor array that had been prepared using the above procedure was incubated for 10 minutes at room temperature with a 1 μM solution of FAM-labeled oligonucleotide that was complementary to the surface-immobilized primer. The microreactor array was then rinsed thoroughly with MilliQ water, and the surface of the array was imaged with an epifluorescence microscope. A fluorescence image of the labeled DNA coating the inner walls of the microreactor array is shown in FIG. 38.

PCR forward primer:
(SEQ ID NO: 15)
5′- CCA TCT CAT CCC TGC GTG TC -3′
PCR forward primer complement:
(SEQ ID NO 16)
5′- GAC ACG CAG GGA TGA GAT GG -3′

### Example 15

In many instances, it is desirable to pattern the PDMS microreactors with a stable monolayer of functionalized silane. In the previous example, trimethoxysilane aldehyde was polymerized on the surface, forming multiple layers. In addition, the aldehyde functionality is relatively unstable. In contrast, 3-aminopropyldiisopropylethoxy silane forms a monolayer on the PDMS surface under mildly basic conditions because of a reduced propensity for polymerization. Additionally, the resulting amino-funetionalized surface is more stable under ambient conditions.

A PDMS microreactor array containing 5-μm holes was fabricated from a silicon master array of 5-μm pillars (in SU-8 photoresist) by pouring Sylgard 184 (10:1 PDMS base to curing agent ratio) on the silicon master and curing overnight at 70° C. The PDMS microreactor array was peeled from the master and sealed to a glass slide, trapping air in the microreactors. The glass slide with sealed microreactors was treated with air plasma for 60 seconds in a plasma sterilizer and then removed from the glass slide.

About 0.2 mL of 3-aminopropyldiisopropylethoxy silane was added to a 5% mixture of water in 200-proof ethanol. The silane was incubated in aqueous ethanol for 10 minutes at room temperature before the plasma treated PDMS microreactor array was submerged in the silane solution. The PDMS microreactor array was incubated in the silane solution for 15 minutes before being dipped briefly in aqueous ethanol in the absence of silane. The PDMS microreactor array was then placed face-up on a heat block at 100° C. for one minute. A 4-μM solution of 5′-benzaldehyde functionalized PCR forward primer in Cyanoborohydride Coupling Buffer (20 mM sodium phosphate pH 7.5, 200 mM sodium chloride, 3 g/L sodium cyanoborohydride, Sigma) was pipetted onto the microreactor array surface, which was placed under vacuum for 2 hours at room temperature. The microreactor array was then rinsed thoroughly with MilliQ water and dried with nitrogen.

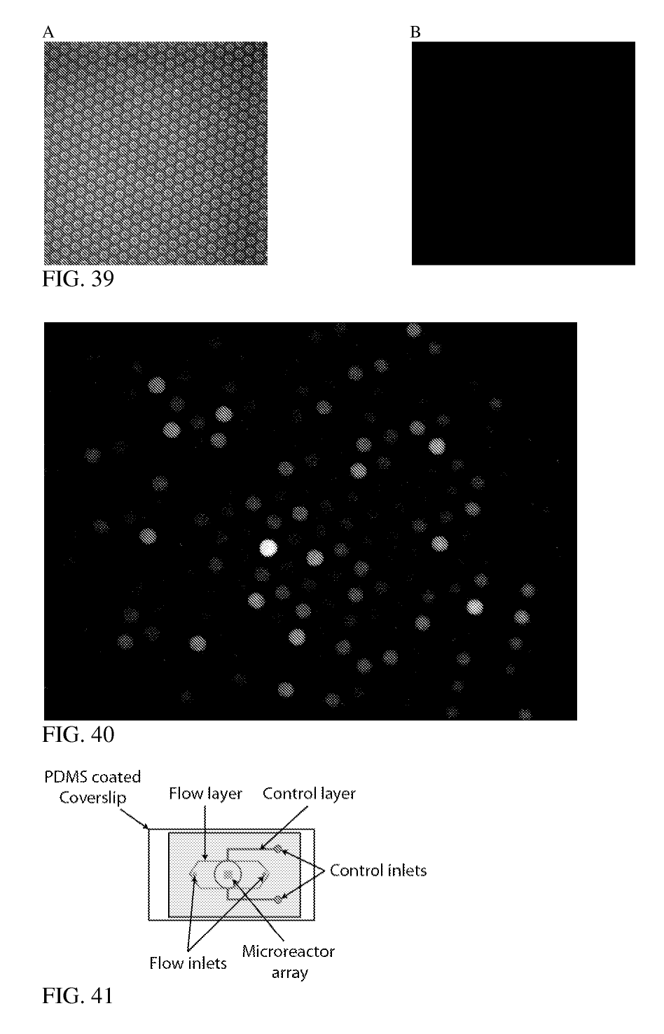

In order to demonstrate covalent patterning of 5′-benzaldehyde-functionalized PCR forward primer on the inner walls of PDMS microreactors, a microreactor array prepared using the above procedure was incubated for 10 minutes at room temperature with a 1-μM solution of FAM-labeled oligonucleotide that was complementary to the surface-immobilized primer. The microreactor array was then rinsed thoroughly with MilliQ water, and the surface of the array was imaged with an epifluorescence microscope. A fluorescence image of the labeled DNA coating the inner walls of the microreactor array is shown in FIG. 39A. This experiment was repeated with a 1-μM solution of FAM-labeled oligonucleotide that was not complementary to the surface-immobilized primer. The resulting epifluorescence image (FIG. 39B) showed no detectable nonspecific hybridization to the microreactor walls.

### Example 16

A 5′-phosphorylated DNA template (Integrated DNA Technologies) was circularized using CircLigase H (Epicentre Biotechnologies) single-stranded DNA ligase. A 500-nM solution of phosphorylated DNA template was incubated in 1× CircLigase II Reaction Buffer (Epicentre Biotechnologies) with 1 M betaine, 2.5 min MnCl₂, and 200 units of CircLigase II for 3 hours at 60° C. The CircLigase II reaction mixture was then treated with Exonuclease I to digest any remaining single-stranded DNA by adding 2.5 fL of Exonuclease I Reaction Buffer (New England BioLabs) and 40 units of Exonuclease I (New England BioLabs) to 20 μL of the circularization reaction mixture. This new reaction mixture was incubated at 37° C. for 2 hours. Both CircLigase II and Exonuclease I were heat inactivated by incubation at 80° C. for 20 minutes.

A 25-nM solution of circularized DNA template was incubated on ice for 10 minutes with 25 nM of reverse PCR primer (Integrated DNA Technologies), which also served as a primer for RCA. To initiate RCA, the primed, circularized template was diluted to 25 pM in 1× Phi29 DNA polymerase Reaction Buffer (New England BioLabs), 1 mM dNTPs, 0.1 mg/mL BSA, and 15 nM Phi29 DNA polymerase (New England BioLabs). The RCA reaction mixture was incubated at 30° C. for 30 minutes prior to heat inactivation of Phi29 DNA polymerase by incubation at 65° C. for 10 minutes.

The RCA product was then diluted to 9 μM in a 1× Taq MasterMix (New England BioLabs) with 0.2% Pluronic F-27, an additional 200 units/μL Taq DNA polymerase (New England BioLabs), 0.1 mg/mL BSA, 0.5 μlVIPCR forward primer, 0.5 μM PCR reverse primer, 0.25 μM TagMan FAM-Zen probe (Integrated DNA Technologies), and 2.4 units/mL Thermostable Inorganic Pyrophosphatase (New England BioLabs).

A multi-layer on-chip PCR microfluidic device was constructed from PDMS as described herein. The device was hydrated for 10 minutes at 92° C. by placing the control layer under 12 psi of water pressure (sealing the microreactor array) while the flow layer was under 6 psi of water pressure. The device was then pre-treated with only the protein components of the PCR mixture by trapping the reaction mixture in the microreactor array and running 30 thermocycles in the absence of DNA. The DNA-containing reaction mixture, including the RCA pre-amplicon, was introduced into the microreactor array, which was then sealed. The device was then run for 5 thermocycles of:

15 s at 92° C.

30 s at 50° C.

30 s at 68° C.

The microreactor array was imaged on an epifluorescence microscope (Nikon TE-300) with a 60×1.4 NA oil-immersion objective (Nikon), a 470 nm LED (Thorlabs), and a CCD camera (CoolSnap, Photometrics). Fluorescence signal was observed above background in less than 1% of the microreactors at this point. After an additional 5 thermocycles, the microreactor array was re-imaged, and fluorescence signal was observed from 20-30% of the microreactors, consistent with Poisson-loading of the microreactors with RCA pre-amplicon (FIG. 40). Rolling circle pre-amplification significantly reduces the number of PCR cycles required to generate signal in the TaqMan assay.

PCR forward primer:
(SEQ ID NO: 17)
5′-CCA TCT CAT CCC TGC GTG TC-3′
PCR reverse primer:
(SEQ ID NO: 18)
5′- CCT ATC CCC TGT GTG CCT TG -3′
Rolling circle template:
(SEQ ID NO: 19)
5′- CCT ATC CCCTGT GTG CCT TGT CAG CTA GGC CCG
AGA AAA ATA GAT GGA GGT GCT CCT GGC GGC TCA TCA
TTA ATA CAC ATG ACA CGC AGG GAT GAG ATG G -3′

### Example 17

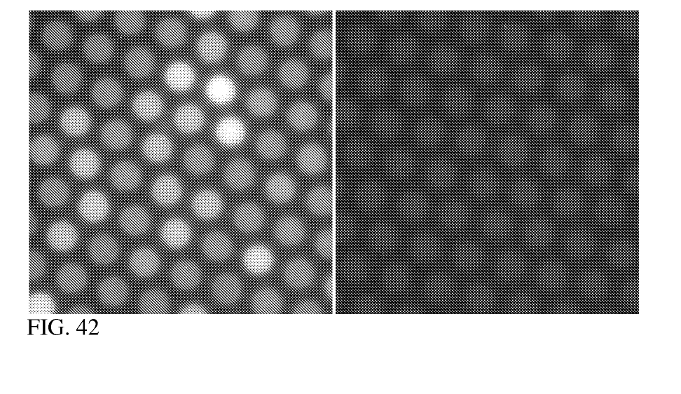

A silicon master for the generation of 5 micron holes was generated using standard photolithographic procedures (as described). Sylgard 184 PDMS was mixed at a ratio of 10:1 prepolymer base: curing agent and degassed under vacuum until all bubbles were removed (approximately 30 minutes). This PDMS was spun to approximately 150 micron thickness on a 3 inch silicon wafer containing SU-8 posts. Additionally, PDMS was spun to approximately 150 micron thickness on a blank, fluorosilanized 3 inch silicon wafer. PDMS was also spun on a clean glass coverslip (which had been plasma oxidized for 4 minutes) to a thickness of approximately 10 microns. Finally, 11 grams of PDMS were poured onto a 3 inch control layer master silicon wafer to create a control layer approximately 2.5 mm thick. All PDMS was cured for at least 1.5 hours at ˜75° C. The control layer was peeled from the silicon master and trimmed, and 0.75-mm diameter inlets were punched. The control layer was then bonded after 1 minute of plasma oxidation to the layer containing the PDMS microarrays, forming a thin membrane across the control valve. These two layers were then bonded to the flow layer, which was cut from the fluorosilanized master using a razor blade. During the plasma oxidation process, the holes were blocked with a 4 mm disk of PDMS to preserve hydrophobicity of the interstitial reactor walls. Finally, these three bonded layers were in turn bonded to the PDMS coverslip after plasma oxidation. Again, a PDMS disk was used to protect the sealing surface of the microreactors to maintain their hydrophobicity, as well as a region of the PDMS coated coverslip directly under the mieroreactor region. Next, 0.75-mm diameter holes were punched in this device to make inlets for the flow layer. Immediately after, trimethoxysilane aldehyde (United Chemical Technologies) in 95% ethanol and 5% dilute acetic acid, which had been incubated for 10 minutes under vacuum, was added to these devices and allowed to incubate for 2 minutes. The devices were then washed with 95% ethanol and 5% dilute acetic acid, heated on a hotplate for 1 minute at 100° C., and dried with dry nitrogen. Then a 10-μM solution of 5′-aminated PCR reverse primer containing an 18 atom PEG spacer in Cyanoborohydride Coupling Buffer (20 mM sodium phosphate pH 7.5, 200 mM sodium chloride, 3 g/L sodium cyanoborohydride, Sigma) was introduced to the flow chamber, and the air in the reactors was eliminated by depressing on the top of the device with a pipette. This solution was incubated for 2 hours and then washed with water, and then the reaction was quenched with 10% ethanolamine in Cyanoborohydride Coupling Buffer for 15 minutes. Finally the device was washed with water and dried with dry nitrogen. A schematic of the device is shown in FIG. 41.

### Example 18

Using the patterned device generated in Example 17, we carried out asymmetric PCR to extend template DNA onto primers covalently immobilized on the walls of the microreactors. In this experiment, the buffer conditions were identical to those in Example 12, except that 125 units/mL Taq DNA polymerase, 500 nM forward primer, and 20 nM reverse primer were used; the Taqman probe was not used; and 2 nM target DNA was used as an amplification target. Prior to loading this reaction mixture, water was loaded into the patterned device, the microreactors were sealed by applying 13 psi pressure, and the flow layer was pressurized to 6 psi. Then, the reactors were heated on a Peltier-based temperature controller to 92° C. to saturate the PDMS with water. After 10 minutes, the device was cooled to room temperature, and the above reaction mixture excluding the DNA components (i.e., primer, probe, and template) was introduced to the flow layer, which was then re-sealed and re-pressurized. The device was then thermocycled for 5 cycles each consisting of 15 s at 92° C., 30 s at 58° C., and 15 s at 68° C. to equilibrate the device further. Finally, the reaction mixture was introduced to the flow layer, which was then re-sealed and re-pressurized. The device was then thermocycled for 12 cycles each consisting of: 15 s at 92° C., 30 s at 58° C., and 30 s at 68° C. The device was then further cycled for 30 cycles using the same parameters, except the annealing step was decreased to 50° C. Then, the device was washed with a buffer consisting of 50 mM Tris-HCl pH 8.0, 50 mM NaCl, 1 mM DTT, 0.1 mM EDTA, and 0.1% Tween-20 (v/v) for 5 minutes while being held at 92° C. to melt the complementary strand from the strand synthesized on the wall of the reactor. Then, 1 micromolar of forward primer was introduced to the reactors in this wash buffer and allowed to anneal at 37° C. for 4 minutes and 25° C. for 4 minutes. This primer was washed out, and the reactors were incubated in 50 mM Tris-HCl pH 8.0, 50 mM NaCl, 1 mM DTT, 0.1% Tween-20 (v/v), and 9.1 nM Klenow Fragment (exo-) for 2 minutes. The device was then cooled to 2° C., and the following reaction mixture was introduced to the reactors: 50 mM Tris-HCl pH 8.0, 50 mM NaCl, 1 mM DTT, 0.5 min MnCl₂, 0.1% Tween-20 (v/v), 13.5% glycerol (v/v), 1 μM dG4P-FAM, 1 μM dC4P-FAM, 3.5 μM dA4P-FAM, 4.5 μM dT4P-FAM, 0.0075 u/μL SAP, and 9.1 nM Klenow Fragment (exo-). This reaction mixture allowed incorporation of all fluorogenic nucleotides and the detection of immobilized DNA on the walls of the device through the generation of fluorescent signal. The device was sealed, then heated to 37° C. for 10 minutes, and imaged with the microscope described in Example 12. The resulting fluorescence image is shown in the left panel of FIG. 42. After the acquisition of this image, the device was opened, resealed, and imaged, resulting in the right panel of FIG. 42, which shows significantly less signal than the left panel. These experiments demonstrate the generation of fluorogenic signal from DNA covalently attached to the PDMS walls through on-chip PCR.

PCR forward primer:
(SEQ ID NO: 20)
5′- CCA TCT CAT CCC TGC GTG TC -3′
PCR reverse primer:
(SEQ ID NO: 21)
5′- CCT ATC CCC TGT GTG CCT TG -3′
Amplification target:
(SEQ ID NO: 22)
5′- CCA TCT CAT CCC TGC GTG TCA TGT GTA TTA ATG
ATG AGC CGC CAG GAG CAC CTC CAT CTA TTT TTC TCG
GGC CTA GCT GAC AAG GCA CAC AGG GGA TAG G -3′

### Example 19

### Preparation of 8-(3′-O-Methyl-4,7,2′,7′-Tetrachloro-5(6)-Carboxyfluorescein-6′-yl)-deoxyadenosine-5′-tetraphosphate (dA4P-TCF)

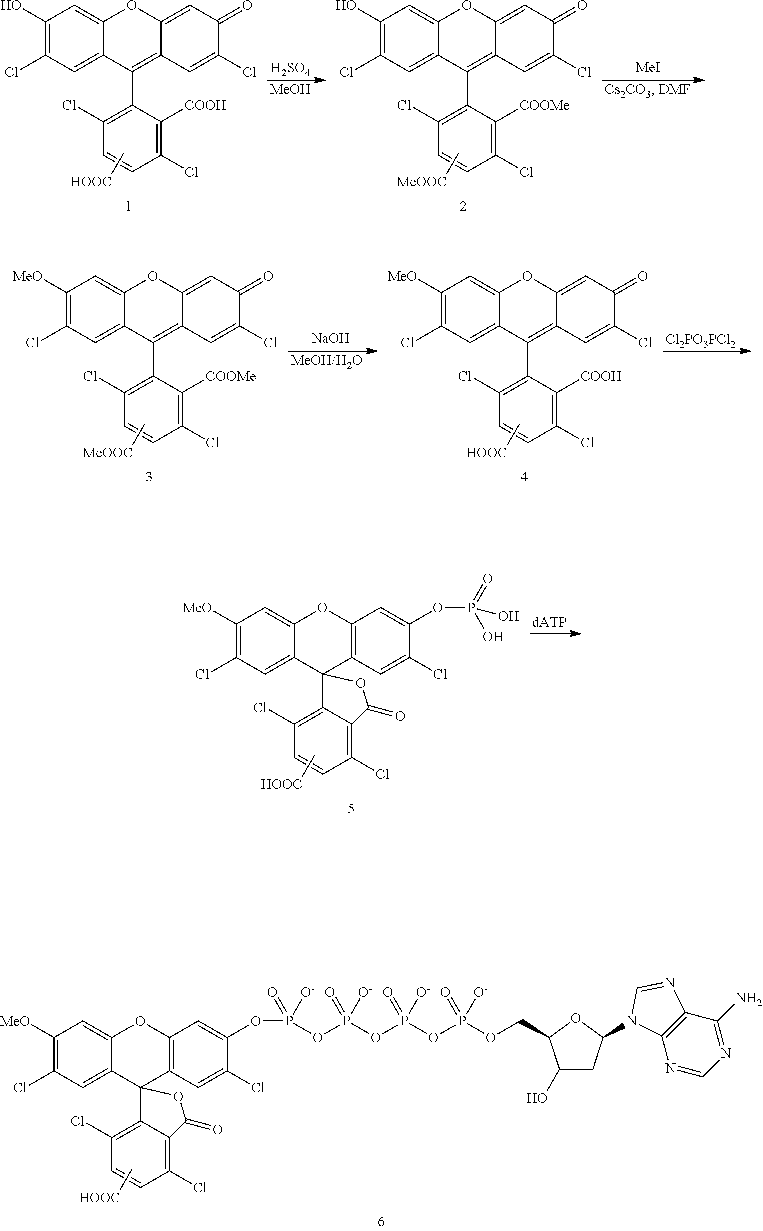

Preparation of TCF monophosphate

4,7,2′,7′-Tetrachloro-5(6)-carboxyfluorescein 1 (1.5 g, 2.90 mmol) was dissolved in methanol (60 mL); then H₂SO₄ (cone. 2 mL) was added dropwise under stirring. The mixture was heated under reflux for 10 hr. After the reaction was complete, the solution was concentrated, diluted with dichloromethane, then washed with sodium phosphate buffer (pH 7.0) and brine, and dried over sodium sulfate. After evaporation of the dichloromethane, the residue was purified by silica gel chromatography to afford compound 2 (940 mg, 60%). MS (ES): M−1=541.81 (calc 541.93)

MeI (604 mg, 4.25 mmol) was added to a solution of diester 2 (920 mg, 1.70 mmol) and cesium carbonate (831 mg, 2.55 mmol) in DMF (25 mL). The reaction mixture was stirred for 2 hr at room temperature. DMF was removed by vacuum pump. The residue was diluted with dichloromethane, then washed with 2N HCl and brine, and dried over magnesium sulfate. The organic phase was concentrated to afford compound 3, which was dissolved in methanol (60 mL) for the next step without further purification.

To the methanol solution, 2N NaOH (5.8 mL) was added, and the mixture was stirred for 8 hr at room temperature. Upon completion of the reaction, the methanol was evaporated, and the aqueous residue was acidified with 2N HCl. The resulting precipitate was collected by filtration and dried to afford crude compound 4 (650 mg, 72%), which can be further purified by silica gel chromatography. ¹H NMR (300 MHz, CD₃OD): δ 3.49 (s, 3H), 6.59 (brs, 2H), 7.02 (s, 2H), 7.82 (s, 1H); MS (ES): M−1: 527.15 (talc 527.92).

3′-O-Methyl-4,7,27-tetrachloro-5(6)-carboxyfluorescein 4 (100 mg, 0.19 mmol) was suspended in acetonitrile (15 mL), and the solution was cooled to 0° C. in an ice bath. Pyrophosphoric chloride (214 mg, 0.85 mmol) was added under stirring at 0° C. The mixture became a clear solution after stirring for 15 min; N,N-diisopropylethylamine (196 mg, 1.52 mmol) was added; and the reaction was stirred for 2 hr at 0° C. The reaction was quenched by adding TEAB buffer (50 mM, 10 mL). After 1 hr, the mixture was concentrated in vacuo and purified by HPLC on an Xterra RP C-18 19-150 mm column using 0-30% acetonitrile in 50 mM TEAB buffer, flow rate 5 mL/min. Fractions containing product were concentrated and coevaporated with anhydrous DMF and tributylamine to produce an anhydrous monophosphate tributylammonium salt, which was used for the next step. HPLC purity at 476 nm >90%, UV/VIS λₘₐₓ=236 nm and 476 nm. MS (ES): M−1=606.86 (talc 606.88).

Preparation of dA4P-TCF

2′-deoxyadenosine-5′-triphosphate disodium salt (6.8 mg, 14.0 μmol) was converted to the tributylammonium salt by treatment with ion-exchange resin (BioRad AG-50W-XB) and tributylamine. After removal of the water, the obtained tributylammonium salt was coevaporated with anhydrous DMF (2 mL) twice and then redissolved in 0.3 mL anhydrous DMF. To the solution, carbonyldiimidazole (CDI, 11.3 mg, 70 μmol, 5 eq) was added, and the mixture was stirred at room temperature for 12 hr (monitored by LCMS). MeOH (3.2 μl) was added, and the solution stirred for 0.5 hr to destroy the excess CDI. The 3′-O-Methyl-TCF monophosphate tributylammonium salt (16 μmol) DMF solution (0.3 mL) from the previous step was transferred into the reaction by syringe, and MgBr₂ (18 mg, 70 μmol, 5 eq) in DMF was also added at the same time. The mixture was stirred for 3 days at room temperature. Then, the reaction mixture was concentrated, diluted with water, filtered, and purified on a Hi-Trap 5 mL ion exchange column (GE Healthcare) using a two step gradient: first water then 50 mM PIPES/1 M NaCl buffer. Fractions containing the product were collected, and shrimp alkaline phosphatase was added to destroy the unreacted monophosphate. After 30 min, the solution was concentrated and repurified by HPLC on an Xterra RP C-18 19-150 mm column (Waters) using 0-30% acetonitrile in 50 mM triethylammonium acetate buffer (PH 7), flow rate 5 mL/min. Fractions containing pure product were concentrated and further purified by a HiTrap 1 mL ion exchange column (GE Healthcare) to give a 0.7 mL of a 1 mM solution. UV/VIS λmax=260 nm and 470 nm. MS (MALDI-TOF): M+1=1083.60 (calc 1083.88)

### Example 20

### Preparation of δ-(3′-O-Methyl-4,7,2′,4′,5′,7′-Hexachloro-5(6)-Carboxyfluorescein-6′-yl)-deoxyadenosine-5′-tetraphosphate (dA4P-δ-HCF)

### I. Preparation of 2,4-dichlororesorcinol

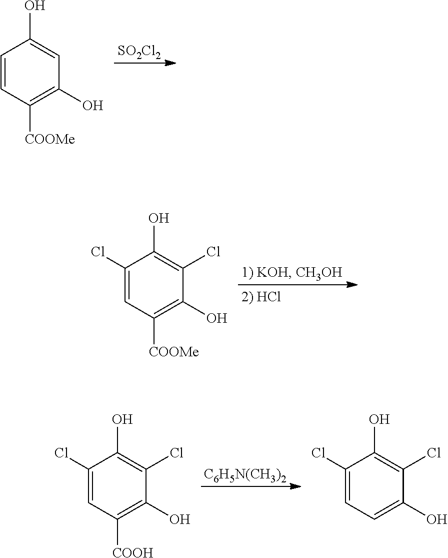

Methyl 2,4-dihydroxybenzoate 25.0 g (0.15 mol) was dissolved in 30 mL SO₂Cl₂, and then the solution was heated slowly to reflux in a fume hood (gas generated). After about 15 minutes, an additional 60 mL SO₂Cl₂ was added to the reaction, which was kept refluxing for an additional 2 h. After the reaction was completed by TLC monitoring, SO₂Cl₂ was removed by rotary evaporation, and the remaining solid was collected and recrystallized by EtOH/H₂O (1/1 mixture). The product methyl-3,5-dichloro-2,4-dihydroxybenzoate was collected by filtration in 60% yield as pale white solid. ¹H NMR (300 MHz, CDCl₃): δ 3.95 (s, 3H), 6.37 (s, 1H), 7.80 (s, 1H), 11.6 (s, 1H).

In a 500 mL round-bottom flask containing 200 mL NaOH (13.0 g) MeOH solution was added 3,5-dichloro-2,4-dihydroxybenzoate (20 g), and the solution was heated to 60° C. under stirring for 8 h. Then the reaction was cooled to room temperature and concentrated to about 50 mL by rotary evaporation. The pH of the solution was adjusted to about 1.0 with concentrated HCl. The solid was collected and recrystallized in EtOH/H₂O(1/1 mixture), and the product 3,5-dichloro-2,4-dihydroxybenzoic acid was obtained as white solid in 75% yield. 1H NMR (300 MHz, D₂O): δ 7.77 (s, 1H), 7.83 (s, 1H).

The 3,5-dichloro-2,4-dihydroxybenzoic acid (5.0 g) was suspended in 10 mL NN-dimethyl aniline, and the mixture was heated slowly to 130° C. (CO₂ gas was evolved at this point). After 10 min, the reaction was heated to 185° C. for 2 h. The reaction was cooled to room temperature and poured into 15 mL cone. HCl at 0° C. with rapid stirring. The mixture was extracted with ethyl ether (30 mL×4), and the combined organic phase was washed with 6 N HCl and brine and dried by MgSO₄. After evaporation of the solvent, the residue was purified by silica gel chromatography to afford 2,4-dichlororesorcinol in 75% yield as white solid. ¹II NMR (300 MHz, CDCl₃): δ 5.50 (s, 1H), 5.83 (s, 1H), 5.58-6.65 (d, 1H), 7.14-7.16 (d, 1H).

### II. Preparation of 3′-O-Methyl-4,7,2′,4′,5′,7′-Hexachloro-5(6)-Carboxyfluorescein (3′-O-Me-HCF)

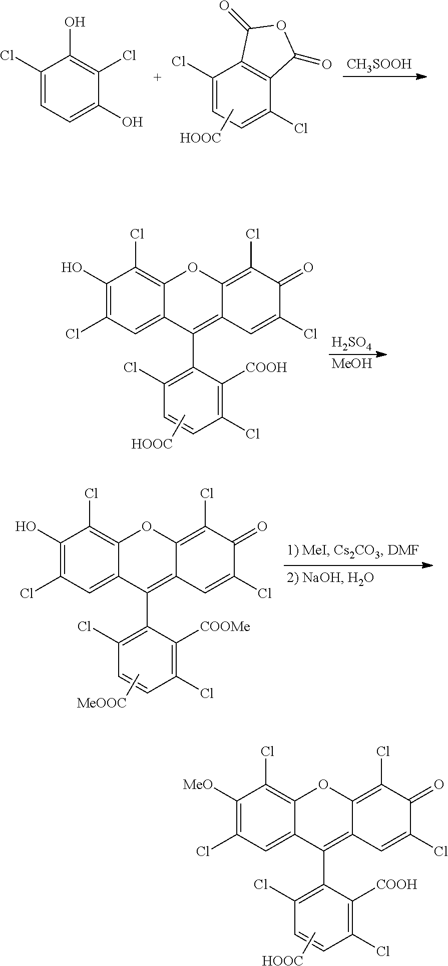

To a flame dried 500 mL round-bottom flask containing 3.47 g (13.0 mmol) of 3,6-dichloro-trimallitic anhydride and 4.97 g (27.8 mmol) 2,4-dichlororesorcinol was added 60 mL of methane sulfonic acid. The mixture was heated for 3 h at 150-160° C. Then, the dark red mixture was cooled and poured slowly into 200 mL of rapidly stirred water. The brown-red solid was collected by suction filtration, washed with 200 mL of water, and dried by oil pump over P₂O₅ to afford the product HCF. Yield: 56%; MS (ES): M+1: 583.02 (calc 581.82).

HCF (4.5 g, 7.7 mmol) was dissolved in methanol (120 mL), and H₂SO₄ (cone. 5 mL) was added dropwise under stirring. The mixture was heated under refluxing for 10 h. After the reaction was completed by TLC monitoring, the solution was concentrated and diluted with dichloromethane, then washed with sodium phosphate buffer (pH 7.0) and brine, and dried over sodium sulfate. After evaporation of the dichloromethane, the residue was purified by silica gel chromatography to afford dimethyl-HCF (55%). MS (ES): M+1: 611.01(calc 609.85).

Dimethyl-HCF was placed in a 250 mL round-bottom flask containing 90 mL DMF and 4.7 g (14.6 mmol) cesium carbonate. To the mixture was added MeI (2.6 g, 18.2 mmol), and the mixture was stirred for 2 h at room temperature. DMF was removed by vacuum pump. The residue was diluted with dichloromethane, then washed with 2N HCl and brine, and dried over magnesium sulfate. The organic phase was concentrated to afford the crude 3′-O-methylated compound, which was dissolved in methanol (60 mL) for next step without further purification.

To the methanol solution, 2N NaOH (20 mL in water) was added, and the mixture was stirred for 8 hr at room temperature. The reaction was monitored by TLC to make sure all starting material was consumed. Then methanol was evaporated, and the aqueous residue was acidified with 2N HCl. The resulting precipitate was collected by filtration and dried to afford compound 3′-O-Me-HCF (61%), which can be further purified by silica gel chromatograph. UV/VIS λ^{max}=253 nm and 537 nm. MS (ES): M−1: 595.03 (calc 595.84).

### III. Synthesis of dA4P-δ-HCF

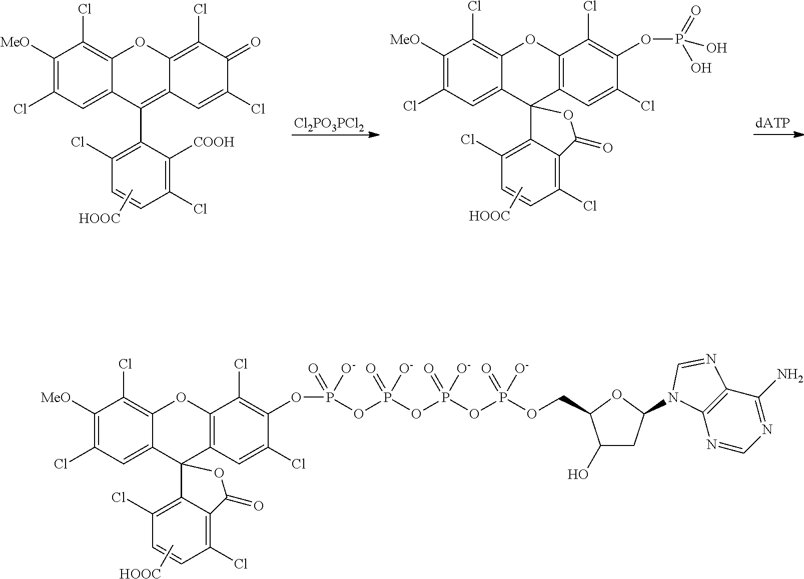

3′-O-Methyl-HCF (3′-O-Methyl-4,7,2′,4′,5′,7′-Hexachloro-5(6)-Carboxyfluorescein) (113 mg, 0.19 mmol) was suspended in acetonitrile (15 mL), and the solution was cooled to 0° C. in an ice bath. Pyrophosphoric chloride (214 mg, 0.85 mmol) was added under stirring at 0° C. The mixture became a clear solution after stirring for 15 min, N,N-diisopropylethylamine (196 mg, 1.52 mmol) was added, and the reaction was stirred for 2 hr at 0° C. The reaction was quenched by adding TEAB buffer (50 mM, 10 mL). After 1 hr, the mixture was concentrated in vacuo and purified by 1-IPLC on an Xterra RP C-18 19-150 mm column (Waters) using 0-30% acetonitrile in 50 mM TEAB buffer, flow rate 5 mL/min. Fractions containing product were concentrated and coevaporated with anhydrous DMF and tributylamine to make an anhydrous monophosphate tributylammonium salt. UV/VIS λₘₐₓ=238 nm and 489 nm. MS (ES): M−1=674.96 (talc 675.80).

2′-deoxyadenosine-5′-triphosphate disodium salt (6.8 mg, 14.0 μmol) was converted to the tributylammonium salt by treatment with ion-exchange resin (Bio-Rad AG-50W-XB) and tributylamine. After removal of the water, the tributylammonium salt was coevaporated with anhydrous DMF (2 mL) twice and redissolved in 0.3 mL anhydrous DMF. To the solution carbonyldiimidazole (CDI, 11.3 mg, 70 μmol, 5 eq) was added, and the mixture was stirred at room temperature for 12 h (monitored by LCMS). After that MeOH (3.2 μl) was added and stirred for 0.5 hr to destroy the excess CDI. Then, the 3′-O-Methyl-HCF monophosphate tributylammonium salt (16 μmol) DMF solution (0.3 mL) from the previous step was transferred into the reaction by syringe, and MgBr₂ (18 mg, 70 μmol, 5 eq) in DMF was added at the same time. The mixture was stirred for 3 days at rt. Then the reaction mixture was concentrated, diluted with water, filtered, and purified on a HiTrap 5 mL ion exchange column (GE Healthcare) using two step gradient: first water then 50 mM PIPES/1 M NaCl buffer. Fractions containing the product were collected, and shrimp alkaline phosphatase (USB Corp.) was added to destroy the unreacted monophosphate. After 30 mM, the solution was concentrated and repurified by HPLC on an Xterra RP C-18 19-150 mm column (Waters) using 0-30% acetonitrile in 50 mM triethylammonium acetate buffer (PH 7), flow rate 5 mL/min. Fractions containing pure product were concentrated and further purified by using a HiTrap 1 mL ion exchange column (GE Healthcare) to give 0.5 mL 0.6 mM solution. UV/VIS λₘₐₓ=241, 387 and 487 nm.

### Example 21

### Preparation of resorufin-4-carboxylic acid

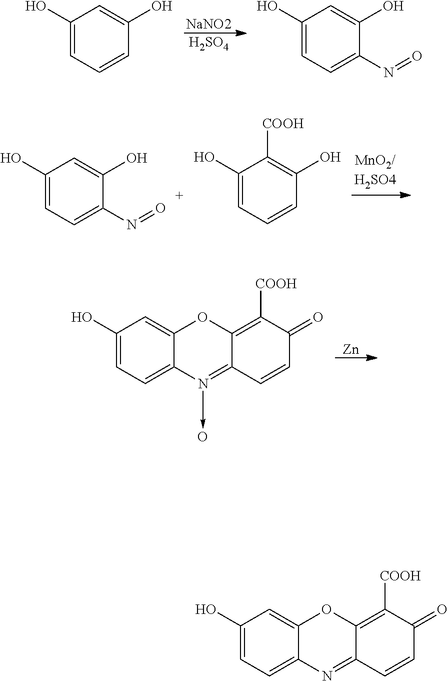

Sulfuric acid (conc. 3.5 mL) was added to a 500 mL flask containing 170 mL of water, which was then cooled to 4° C. in an ice bath. Resorcinol (7.2 g, 65 mmol) was then added under stirring. After 5 min, a sodium nitrite (5.4 g, 78 mmol) water solution was added slowly. The temperature was kept around 5-8° C. for 30 min and then allowed to warm to 20° C. for another 30 min. The reaction was diluted with 200 mL water, and the precipitated product was collected by suction filtration, washed with water, and dried by vacuum pump to give a yellow product (4-nitrosoresorcinol in 75% yield).

4-nitrosoresorcinol (3.9 g, 28 mmol) was dissolved in 80 mL of methanol with sonication. The resultant solution was cooled to 4° C. using an ice-water bath. 2,6-Dihydroxy benzoic acid (4.25 g, 28 mmol) was added in one portion and followed by MnO₂ (2.5 g, 28 mmol) with stirring. Concentrated sulfuric acid (3.1 mL) was added within 5 min at 0-4° C. with intensive stirring. The resultant mixture was stirred at room temperature for 4 h and then diluted with ethyl ether (100 mL). The precipitated material was collected by suction filtration, washed with MeOH/ethyl ether (1:1) mixture, and dried. This solid was re-dissolved in a mixture of 100 mL water and 25 mL 30% NH₄OH aqueous solution and filtered and washed with water. The filtration was cooled to 0° C. using ice bath, and then zinc powder (18.0 g, 0.28 mol) was added with rapid stirring. The reaction was monitored by TLC (developing solvent: ethyl acetate/methanol 5/1). After 1 h, the reaction solution was acidified by concentrated HCl to pH ˜2-3. The precipitated brown solid was collected by filtration, washed with water (200 mL), and then dried under vacuum. Yield: 20%. UV/VIS λₘₐₓ=241 nm and 570 nm. ¹H NMR (500 MHz, CD₃OD/DMSO-d6): δ 7.77 (d, J=9.0 Hz, 1H), 7.59 (d, J=9.5 Hz, 1H), 6.95 (d, J=9.0 Hz, 1H), 6.87 (d, J=9.5 Hz, 1H). 6.56 (s, 1H); MS (ES): M+1: 258.11 (calc 257.03).

### Preparation of resorufin-4-carboxylic acid monophosphate

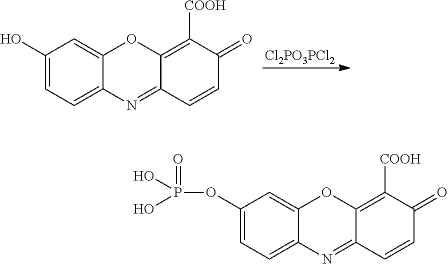

Resorufin-4-carboxylic acid (50 mg, 0.19 mmol) was suspended in acetonitrile (8 mL), and then the solution was cooled to 0° C. in ice bath. Pyrophosphoric chloride (214 mg, 0.85 mmol) was added under stirring at 0° C. After 15 min, DBU (1,8-Diazabicyclo-[5,4,0]-undec-7-ene)(231 mg, 1.52 mmol) was added, and the reaction was stirred for further 2 hr at 0° C. The reaction was quenched by adding TEAB buffer (50 mM, 10 mL). After 1 hr, the mixture was concentrated in vacuo and purified by HPLC (Xterra RP C-18 19-150 mm column, Waters) using 0-30% acetonitrile in 50 mM TEAB buffer, flow rate 5 mL/min. Fractions containing product were concentrated and coevaporated with anhydrous DMF and tributylamine to make a anhydrous monophosphate tributylammonium salt, which was ready for next step in the synthesis. UV/VIS λₘₐₓ=235 nm and 476 nm. MS (ES): M+1=338.21 (calc 337.00).

### Synthesis of dA4P-resorufin-4-carboxylic acid

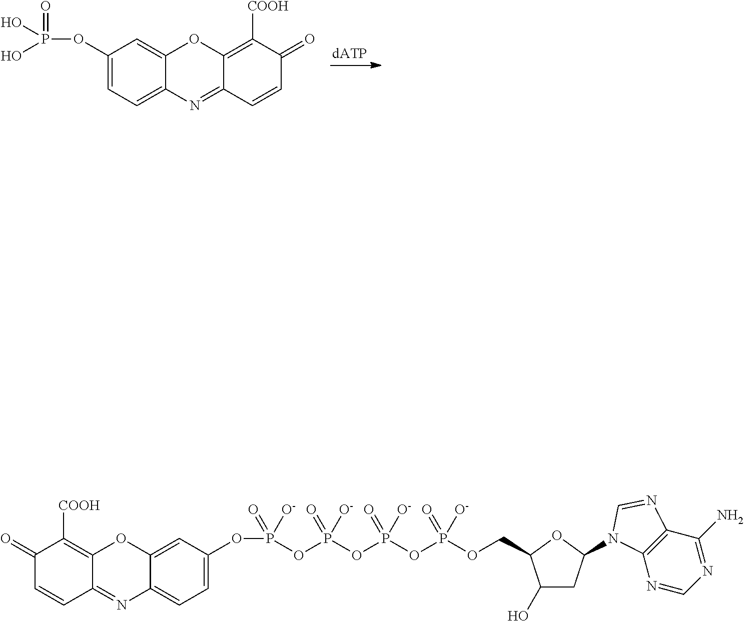 [SMILES: O=C(O)c1c2oc3cc(OP(=O)(O)O)ccc3nc-2ccc1=O ; Nc1ncnc2c1ncn2[C@H]1CC(O)[C@@H](COP(=O)([O-])OP(=O)([O-])OP(=O)([O-])OP(=O)([O-])Oc2ccc3nc4ccc(=O)c(C(=O)O)c-4oc3c2)O1]

2′-deoxyadenosine-5′-triphosphate disodium salt (7.0 mg, 14.2 μmol) was converted to a tributylammonium salt by treatment with ion-exchange resin (Bio-Rad AG-50W-XB) and tributylamine. After removal of the water, the obtained tributylammonium salt was coevaporated with anhydrous DMF (2 mL) twice and then redissolved in 0.3 mL anhydrous DMF. Carbonyldiimidazole (CDI, 11.5 mg, 71.1 μmol, 5 eq) was added to this solution, and the mixture was stirred at room temperature for 12 h. After that MeOH (2.8 μl) was added and stirred for 0.5 hr to destroy the excess CDI. Then, resorufin-4-carboxylic acid monophosphate (28 μmol) DMF solution (0.3 mL) from the previous step was transferred into the reaction by syringe, and MgBr₂ (25 mg, 70 μmol, 8 eq) in DMF was also added at the same time. The mixture was stirred for 3 days at rt. Then, the reaction mixture was concentrated, diluted with 50 mM TEAB buffer, filtered, and purified by HPLC (Xterra RP C-18 19-150 mm column, Waters) using 0-30% acetonitrile in 50 mM triethylammonium acetate buffer (pH 7), flow rate 5 mL/min. The fraction containing pure product was concentrated and further purified by a Hi-Trap anion exchange column (GE Healthcare) to give a 0.5 mL, 0.5 min solution. UV/VIS λₘₐₓ=258, 378 and 476 nm. MS (MALDI-TOF) M+1=811.07 (calc 809.99).

### Other Embodiments

All publications, patents, and patent applications mentioned in this specification are herein incorporated by reference to the same extent as if each independent publication or patent application was specifically and individually indicated to be incorporated by reference. While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

Other embodiments are in the claims.

What is claimed is:

1. A method for sequencing a nucleic acid, said method comprising the steps of:
a) immobilizing in an optionally sealed microreactor a single target nucleic acid or a plurality of copies of the target nucleic acid;
b) introducing to the microreactor a mixture in solution phase comprising a nucleic acid replicating catalyst, and a single species of nucleotide comprising a first base and a first label that is substantially non-fluorescent until after incorporation of said nucleotide into a nucleic acid based on complementarity to said target nucleic acid;
c) allowing template-dependent replication of said target nucleic acid or the plurality of copies of said target nucleic acid; and
d) sequencing said target nucleic acid by detecting incorporation of said nucleotide during template-dependent replication by detecting fluorescence emission resulting from said first label.

2. The method of claim 1, wherein said mixture in solution phase further comprises an activating enzyme that renders said first label fluorescent.

3. (canceled)

4. (canceled)

5. (canceled)

6. (canceled)

7. The method of claim 1, wherein said mixture in solution phase further comprises non-hydrolyzable nucleotides that compete for binding to the nucleic acid replicating catalyst to prevent misincorporation of the nucleotide.

8. The method of claim 1, wherein, subsequent to step (d), a second mixture in solution phase comprising an unlabeled nucleotide species comprising the first base is introduced into the microreactor and template-dependent replication is allowed to proceed until the sequencing cycle is complete.

9. (canceled)

10. The method of claim 1, wherein steps (b)-(d) are repeated with a second single nucleotide species comprising a second base and a second label that is substantially non-fluorescent until incorporation of said second nucleotide into said nucleic acid based on complementarity to said target nucleic acid, wherein the first and second labels are the same or different, and the first and second bases are different.

11. (canceled)

12. (canceled)

13. (canceled)

14. The method of claim 1, wherein the microreactor is sealed.

15. (canceled)

16. (canceled)

17. (canceled)

18. The method of claim 1, wherein said nucleic acid replicating catalyst is a DNA polymerase, RNA polymerase, ligase, reverse transcriptase, or RNA-dependent RNA polymerase.

19. (canceled)

20. (canceled)

21. (canceled)

22. The method of claim 1, wherein said target nucleic acid or plurality of copies is immobilized on a bead disposed in said microreactor.

23. The method of claim 1, wherein said plurality of copies is immobilized in step (a).

24. (canceled)

25. (canceled)

26. (canceled)

27. (canceled)

28. (canceled)

29. (canceled)

30. (canceled)

31. (canceled)

32. The method of claim 1, wherein the single species of nucleotide further comprises a reversible terminator.

33. (canceled)

34. (canceled)

35. The method of claim 1, wherein the mixture in solution phase further comprises an exonuclease, wherein a plurality of first labels are produced as a result of incorporation of the nucleotide and subsequent excision by the exonuclease.

36. (canceled)

37. (canceled)

38. (canceled)

39. The method of claim 1, further comprising, prior to step (a), introducing said target nucleic acid, which is reversibly bound to a bead, into said microreactor.

40. The method of claim 1, wherein, in step (a), (i) said microreactor comprises bound oligonucleotides, (ii) a nucleic acid complementary to said target nucleic acid and reversibly bound to a bead is introduced into said microreactor, wherein said complementary nucleic acid binds to one of said bound oligonucleotides, and (iii) said bound oligonucleotide is extended via template-dependent replication, thereby immobilizing said target nucleic acid in said microreactor.

41. (canceled)

42. (canceled)

43. The method of claim 1, wherein, prior to step (b), the microreactor is cooled to 15° C. or lower.

44. (canceled)

45. The method of claim 1, further comprising a population of single target nucleic acids or a population of pluralities of copies of the target nucleic acids, wherein each single target nucleic acid or plurality of copies of the target nucleic acid is immobilized in one of a plurality of microreactors, and steps (b)-(d) are performed for the population.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. A method for sequencing a nucleic acid, said method comprising the steps of:
a) immobilizing in a microreactor a single target nucleic acid or a plurality of copies of the target nucleic acid;
b) cooling said microreactor to 15° C. or lower;
c) introducing to the microreactor a mixture in solution phase comprising a nucleic acid replicating catalyst, and a single species of nucleotide comprising a first base and a first label that is substantially non-fluorescent until after incorporation of said nucleotide into a nucleic acid based on complementarity to said target nucleic acid;
d) sealing said microreactor and heating said microreactor to 20° C. or higher;
e) allowing template-dependent replication of said target nucleic acid or the plurality of copies of said target nucleic acid;
f) sequencing said target nucleic acid by detecting incorporation of said nucleotide during template-dependent replication by detecting fluorescence emission resulting from said first label;
g) repeating steps b)-f) sequentially with a second single nucleotide species comprising a second base and a second label that is substantially non-fluorescent until incorporation of said second nucleotide into said nucleic acid based on complementarity to said target nucleic acid, a third single nucleotide species comprising a third base and a third label that is substantially non-fluorescent until incorporation of said third nucleotide into said nucleic acid based on complementarity to said target nucleic acid; and a fourth single nucleotide species comprising a fourth base and a fourth label that is substantially non-fluorescent until incorporation of said fourth nucleotide into said nucleic acid based on complementarity to said target nucleic acid, wherein any two of the first, second, third and fourth labels are the same or different, and the first, second, third, and fourth bases are different.

59. A method of amplifying a nucleic acid, said method comprising the steps of:
a) providing a single copy of a first nucleic acid having first and second ends;
b) immobilizing the first nucleic acid via the first end to a bead;
c) immobilizing the second end of the nucleic acid to a surface of a microreactor; and
d) amplifying the first nucleic acid to produce a plurality of amplicons having first and second ends, wherein the plurality of amplicons binds to the surface of the microreactor via the second ends or to the bead via the first ends; or
a) providing a single copy of a first nucleic acid having first and second ends;
b) immobilizing the second end of the nucleic acid to a surface of a microreactor; and
c) amplifying the first nucleic acid to produce a plurality of amplicons having first and second ends, wherein the plurality of amplicons binds to the surface of the microreactor via the second ends; or
a) providing a single copy of a first nucleic acid having first and second ends;
b) optionally immobilizing the first nucleic acid via the first end to a bead;
c) immobilizing the second end of the first nucleic acid to one of a plurality of complementary oligonucleotides bound to a surface of a microreactor;
d) extending the oligonucleotide by template dependent replication to produce a second nucleic acid bound to the surface of the microreactor; and
e) amplifying the second nucleic acid to produce a plurality of amplicons extended from said plurality of oligonucleotides bound to said surface of said microreactor; or
a) providing a single copy of a first circular nucleic acid;
b) immobilizing the first nucleic acid to one of a plurality of complementary oligonucleotides bound to a surface of a microreactor or a bead;
c) extending the oligonucleotide by rolling circle amplification to produce a second nucleic acid bound to the surface of the microreactor or bead; and
d) amplifying the second nucleic acid to produce a plurality of amplicons extended from said plurality of oligonucleotides bound to said surface of said microreactor.

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. A system for sequencing a nucleic acid comprising:
a plurality of microreactors that are each capable of holding an immobilized single target nucleic acid or plurality of copies of said target nucleic acid, a mixture in solution phase of a nucleic acid replicating catalyst, and a single species of nucleotide that comprises a label that is substantially non-fluorescent until after incorporation of at least one nucleotide into a nucleic acid based on complementarity to said target nucleic acid;
a fluorescent microscope for imaging said plurality of microreactors to sequence target nucleic acids in said microreactors by detecting in each microreactor the incorporation of an individual nucleotide species during template-dependent replication of said single copy of said target nucleic acid by monitoring fluorescence from said labels resulting from incorporation of said at least one nucleotide; and
a fluidic delivery system capable of delivering liquids from each of four reservoirs to each of said plurality of microreactors.

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. A compound selected from the group consisting of formula: 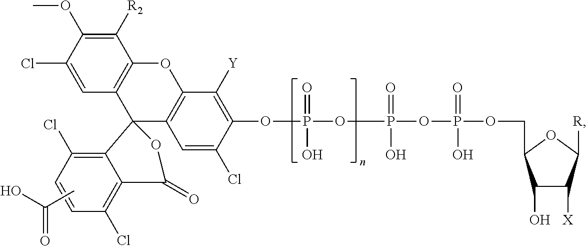
wherein n is 0 to 4, R is a nucleoside base, X is H, OH, or OMe, and Y is H or Cl, or a salt thereof; and 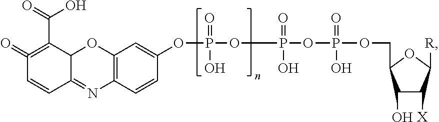
wherein n is 0 to 4, R is a nucleoside base, and X is H, OH, or OMe, or a salt thereof.

114. (canceled)

115. A kit comprising:
a plurality of microreactors that are each capable of holding an immobilized single target nucleic acid, a mixture in solution phase of reagents for template dependent replication of the single target nucleic acid, and a bead functionalized to bind to the single target nucleic acid;
a plurality of beads that are each capable of binding a nucleic acid and being disposed within one of the microreactors; and
reagents for template dependent replication of the nucleic acid.

116. (canceled)

117. (canceled)

118. (canceled)

119. (canceled)
